(19) United States
(12) Patent Application Publication
(10) Pub. No.: US 2022/0288058 A1
(43) Pub. Date: Sep. 15, 2022

(54) COMBINATION THERAPY USING A MALT1 INHIBITOR AND A BTK INHIBITOR

(71) Applicant: Janssen Pharmaceutica NV, Beerse (BE)

(72) Inventors: Anthony T. GREWAY, Bound Brook, NJ (US); Ulrike PHILIPPAR, Antwerp (BE); Bieke VERBIST, Zandhoven (BE); Thomas C. WILDE, Philadelphia, PA (US); Marie E. WILLEMIN, Antwerpen (BE); Yusri A. ELSAYED, Princeton, NJ (US); John GERECITANO, Spring House, PA (US)

(21) Appl. No.: 17/683,470

(22) Filed: Mar. 1, 2022

Related U.S. Application Data
(60) Provisional application No. 63/155,824, filed on Mar. 3, 2021.

(51) Int. Cl. A61K 31/4709; A61K 31/519

(52) U.S. Cl. CPC A61K 31/4709; A61K 31/519

(57) ABSTRACT

The invention relates to a method of treating a disorder or condition that is affected by the inhibition of MALT1 in a subject in need of treatment, comprising administering a BTK inhibitor and a therapeutically effective dose of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

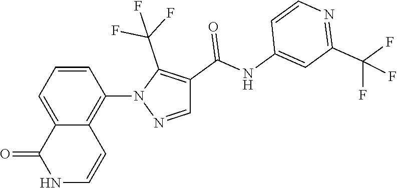 [SMILES: O=C(Nc1ccnc(C(F)(F)F)c1)c1cnn(-c2cccc3c(=O)[nH]ccc23)c1C(F)(F)F]

or a solvate or pharmaceutically acceptable salt form thereof to said subject, wherein said therapeutically effective dose is defined in the specification.

### CLAIM OF PRIORITY

This application claims priority to U.S. Provisional Application No. 63/155,824 filed on Mar. 3, 2021 titled “COMBINATION THERAPY USING A THERAPEUTICALLY EFFECTIVE DOSE OF 1-(1-OXO-1,2-DIHYDROISOQUINOLIN-5-YL)-5-(TRIFLUOROMETHYL)-N-(2-(TRIFLUOROMETHYL)PYRIDIN-4-YL)-1H-PYRAZOLE-4-CARBOXAMIDE AND A BTK inhibitor” which is incorporated herein by reference.

### FIELD OF THE INVENTION

The present invention relates to a method of treating a disease, syndrome, condition, or disorder in a subject, including a mammal and/or human in which the disease, syndrome, condition, or disorder is affected by the inhibition of MALT1, including but not limited to, cancer and/or immunological diseases, by administering to such subject a BTK inhibitor and 1-(1-oxo-1,2-dihydroisoquinolin-5-yl)-5-(trifluoromethyl)-N-[2-(trifluoromethyl)pyridin-4-yl]-1H-pyrazole-4-carboxamide, or a solvate or pharmaceutically acceptable salt form thereof.

### BACKGROUND OF THE INVENTION

MALT1 (mucosa-associated lymphoid tissue lymphoma translocation 1) is a key mediator of the nuclear factor kappa-light-chain-enhancer of activated B cells (NF-κB) signaling pathway and has been shown to play a critical role in different types of lymphoma, including activated B cell-like (ABC) subtype of diffuse large B-cell lymphoma (DLBCL). MALT1 is the only human paracaspase that transduces signals from the B cell receptor (BCR) and T cell receptor (TCR). MALT1 is the active subunit of the CBM complex which is formed upon receptor activation. The “CBM complex” consists of multiple subunits of three proteins: CARD11 (caspase recruitment domain family member 11), BCL10 (B-cell CLL/Lymphoma 10), and MALT1.

MALT1 affects NF-κB signaling by two mechanisms: firstly, MALT1 functions as a scaffolding protein and recruits NF-κB signaling proteins such as TRAF6, TAB-TAK1 or NEMO-IKKα/β; and secondly, MALT1, as a cysteine protease, cleaves and thereby deactivates negative regulators of NF-κB signaling, such as RelB, A20 or CYLD. The ultimate endpoint of MALT1 activity is the nuclear translocation of the FKB transcription factor complex and activation of FKB signaling (Jaworski et al., Cell Mol Life Science 2016. 73, 459-473).

Non-Hodgkin lymphoma represents a diverse set of diseases, of which more than 60 subtypes have been identified (https://www.cancer.net/cancer-types/lymphoma-non-hodgkin/subtypes). Worldwide, DLBCL represents the most common subtype of NHL, accounting for 30% to 40% of all newly diagnosed cases (Sehn L H, Gascoyne R D. Blood. 2015; 125(1):22-32). DLBCL typically presents as an aggressive lymphoma, evolving over months and resulting in symptomatic disease that is fatal without treatment (Ibid).

Constitutive activation of NF-κB signaling is the hallmark of ABC-DLBCL (Diffuse Large B Cell Lymphoma of the Activated B Cell-like subtype), the more aggressive form of DLBCL. DLBCL is the most common form of non-Hodgkin's lymphoma (NHL), accounting for approximately 25% of lymphoma cases while ABC-DLBCL comprises approximately 40% of DLBCL. NF-κB pathway activation is driven by mutations of signaling components, such as CD79A/B, CARD11, MYD88 or A20, in ABC-DLBCL patients (Staudt, Cold Spring Harb Perspect Biol 2010, June; 2(6); Lim et al., Immunol Rev 2012, 246, 359-378).

Outcomes in DLBCL have improved dramatically over the last decade with the addition of rituximab to cyclophosphamide, doxorubicin, vincristine, and prednisone (R CHOP). This regimen remains the current standard of care. However, R CHOP treatment fails in about 30% to 50% of patients with DLBCL (Coiffier B, et al. Hematology Am Soc Hematol Educ Program. 2016; 2016(1):366-378). Less than half of these patients can be cured with stem cell transplantation (Gisselbrecht et al. J Clin Oncol. 2010; 28(27): 4184-4190), and those who are not cured will typically die from their disease (Crump M, et al. Blood. 2017; 130(16):1800-1808). Since the best chance for cure is front-line treatment, there have been many attempts to improve upon R CHOP but so far, these treatments have failed to significantly improve outcomes (Goy A. J Clin Oncol. 2017; 35(31):3519-3522). Recently, several studies have explored the addition of targeted agents to R CHOP in front-line treatment. Promising signs of activity in some of these studies encourage the further exploration of combinations that may improve cure rate of targeted agents in select patients (Chiappella A, et al. Hematological Oncology. 2017; 35(S2):419-428; Younes A, et al. Lancet Oncol. 2014; 15(9):1019-1026). Thus, optimization of front-line therapy, as well as the development of more effective salvage strategies, remains an important objective.

Follicular lymphoma (FL), mucosa-associated lymphoid tissue (MALT) lymphoma, chronic lymphocytic leukemia (CLL), small lymphocytic lymphoma (SLL), mantle cell lymphoma (MCL) and Waldenström macroglobulinemia (WM) are considered largely incurable lymphomas that require therapies throughout the course of disease. Currently, there are limited lines of therapy available for these diseases, and treatments are needed that avoid the use of cytotoxic chemotherapy.

The use of BTK inhibitors, for example Ibrutinib, provides clinical proof-of-concept that inhibiting NF_{K}B signaling in ABC-DLBCL is efficacious. MALT1 is downstream of BTK in the NF_{K}B signaling pathway and a MALT1 inhibitor could target ABC-DLBCL patients not responding to Ibrutinib, mainly patients with CARD11 mutations, as well as treat patients that acquired resistance to Ibrutinib.

Small molecule tool compound inhibitors of MALT1 protease have demonstrated efficacy in preclinical models of ABC-DLBCL (Fontan et al., Cancer Cell 2012, 22, 812-824; Nagel et al., Cancer Cell 2012, 22, 825-837). Interestingly, covalent catalytic site and allosteric inhibitors of MALT1 protease function have been described, suggesting that inhibitors of this protease may be useful as pharmaceutical agents (Demeyer et al., Trends Mol Med 2016, 22, 135-150).

The chromosomal translocation creating the API2-MALT1 fusion oncoprotein is the most common mutation identified in MALT (mucosa-associated lymphoid tissue) lymphoma. API2-MALT1 is a potent activator of the NF_{K}B pathway (Rosebeck et al., World J Biol Chem 2016, 7, 128-137). API2-MALT1 mimics ligand-bound TNF receptor and promotes TRAF2-dependent ubiquitination of RIP1 which acts as a scaffold for activating canonical NF_{K}B signaling. Furthermore, API2-MALT1 has been shown to cleave and generate a stable, constitutively active fragment of NF_{K}B-inducing kinase (NIK) thereby activating the non-canonical F_{K}B pathway (Rosebeck et al., Science, 2011, 331, 468-472).

In addition to lymphomas, MALT1 has been shown to play a critical role in innate and adaptive immunity (Jaworski M, et al., Cell Mol Life Sci. 2016). MALT1 protease inhibitor can attenuate disease onset and progression of mouse experimental allergic encephalomyelitis, a mouse model of multiple sclerosis (McGuire et al., J. Neuroinflammation 2014, 11, 124). Mice expressing catalytically inactive MALT1 mutant showed loss of marginal zone B cells and BIB cells and general immune deficiency characterized as decreased T and B cell activation and proliferation. However, those mice also developed spontaneous multi-organ autoimmune inflammation at the age of 9 to 10 weeks. It is still poorly understood why MALT1 protease dead knock-in mice show a break of tolerance while conventional MALT1 KO mice do not. One hypothesis suggests the unbalanced immune homeostasis in MALT1 protease dead knock-in mice may be caused by incomplete deficiency in T and B cell but severe deficiency of immunoregulatory cells (Jaworski et al., EMBO J. 2014; Gewies et al., Cell Reports 2014; Bornancin et al., J. Immunology 2015; Yu et al., PLOS One 2015). Similarly, MALT deficiency in humans has been associated with combined immunodeficiency disorder (McKinnon et al., J. Allergy Clin. Immunol. 2014, 133, 1458-1462; Jabara et al., J. Allergy Clin. Immunol. 2013, 132, 151-158; Punwani et al., J. Clin. Immunol. 2015, 35, 135-146). Given the difference between genetic mutation and pharmacological inhibition, a phenotype of MALT1 protease dead knock-in mice might not resemble that of patients treated with MALT1 protease inhibitors. A reduction of immunosuppressive T cells by MALT1 protease inhibition may be beneficial to cancer patients by potentially increasing antitumor immunity.

Thus, MALT1 inhibitors may provide a therapeutic benefit to patients suffering from cancer and/or immunological diseases. MALT1 inhibition can be effective in the treatment of ABC DLBCL and other DLBCL subtypes, MALT lymphoma, as well as CLL, MCL, and WM tumors, including tumors that are resistant to a Bruton tyrosine kinase inhibitor (BTKi).

In addition, MALT1 inhibitors used together with a BTKi may provide a therapeutic benefit to patients suffering from cancers and/or immunological diseases. Nagel et al. determined that “[c]ombined inhibition of BTK by Ibrutinib and MALT1 by S-Mepazine additively impaired MALT1 cleavage activity and expression of NF-κB pro-survival factors. Thereby, combinatorial Ibrutinib and S-Mepazine treatment enhanced killing of CD79 mutant ABC DLBCL cells.” Nagel et al., Oncotarget 2015, 6, 42232-42242 at Abstract. Specifically, Nagel et al. observed that “[i]n contrast to the synergistic effects observed for instance by the combination of BTK and PI3K-AKT inhibitors [24], BTK and MALT1 co-treatment yielded additive effects on MALT1 activity and killing of CD79 mutant ABC DLBCL cells. It confirms that both inhibitors are primarily targeting pathological BCR-NFκB signaling.” Nagel et al., Oncotarget 2015, 6, 42239-42240. However, while BTK inhibitor ibrutinib has shown beneficial anti-tumor effects in many B cell malignancies, resistance may occur (Shah et al. Trends Cancer. 2018; 4:197-206) necessitating the development of further combination therapies to improve anti-tumor activity.

### SUMMARY OF THE INVENTION

The present invention relates to a method of treating a disorder or condition that is affected by the inhibition of MALT1 in a subject in need of treatment, comprising administering a therapeutically effective dose ranging from about 25 to 1000 mg, alternatively about 100 to 1000 mg of BTK inhibitor and a therapeutically effective dose ranging from about 25 to 1000 mg, alternatively about 100 to 1000 mg of MALT1 inhibitor 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

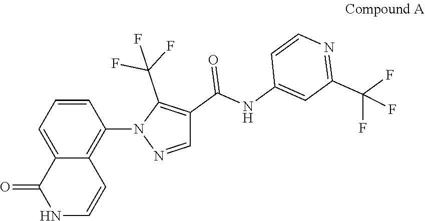 [SMILES: O=C(Nc1ccnc(C(F)(F)F)c1)c1cnn(-c2cccc3c(=O)[nH]ccc23)c1C(F)(F)F]

or an enantiomer, diastereomer, a solvate or pharmaceutically acceptable salt form thereof to said subject. In certain embodiments, the disorder or condition that is affected by the inhibition of MALT1 is also affected by the inhibition of BTK. In some embodiments, the combination of Compound A and BTK inhibitor has synergistic effect in treating the subject.

The BTK inhibitor may be a compound of Formula (I):

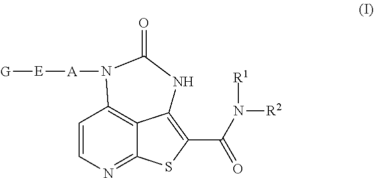

or an enantiomer, diastereomer, solvate or pharmaceutically acceptable salt form thereof. In certain embodiments, the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide (Compound B).

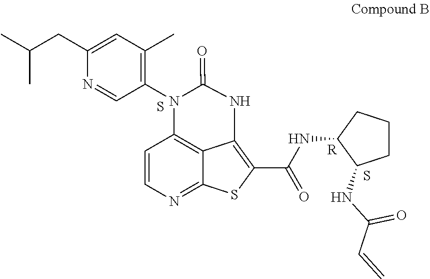

The present invention also relates to a therapeutically effective dose ranging from about 25 to 1000 mg, alternatively from about 50 to 1000 mg, alternatively about 100 to 1000 mg of Compound A or pharmaceutically acceptable salt form thereof and a therapeutically effective dose ranging from about 25 to 1000 mg, alternatively about 100 to 1000 mg of BTK inhibitor for use in treating a disorder or condition that is affected by the inhibition of MALT1. In addition, the present invention relates to use of a therapeutically effective dose ranging from about 50 to 1000 mg, alternatively about 100 to 1000 mg of Compound A or a pharmaceutically acceptable salt form thereof and a therapeutically effective dose ranging from about 50 to 1000 mg, alternatively about 100 to 1000 mg of BTK inhibitor or a pharmaceutically acceptable salt for treating a disorder or condition that is affected by the inhibition of MALT1. Additionally, the present invention relates to use of a therapeutically effective dose ranging from about 50 to 1000 mg, alternatively about 100 to 1000 mg of Compound A or a pharmaceutically acceptable salt form thereof and a therapeutically effective dose ranging from about 50 to 1000 mg, alternatively about 100 to 1000 mg of BTK inhibitor in the manufacture of a medicament for treating a disorder or condition that is affected by the inhibition of MALT1.

The present invention also relates to a therapeutically effective dose ranging from about 25 to 100 mg, alternatively from about 50 to 1000 mg of Compound B or pharmaceutically acceptable salt and a therapeutically effective dose ranging from about 25 to 100 mg, alternatively from about 50 to 1000 mg of Compound A or a pharmaceutically acceptable salt form thereof for use in treating a disorder or condition that is affected by the inhibition of MALT1.

In addition, the present invention relates to use of a therapeutically effective dose ranging from about 25 to 100 mg, alternatively from about 50 to 1000 mg of Ibrutinib and a therapeutically effective dose ranging from about 25 to 1000 mg, alternatively ranging from about 50 to 1000 mg of Compound A or a pharmaceutically acceptable salt form thereof for treating a disorder or condition that is affected by the inhibition of MALT1. Additionally, the present invention relates to use of a therapeutically effective dose ranging from about 25 to 100 mg, alternatively from about 50 to 1000 mg of Ibrutinib and a therapeutically effective dose ranging from about 50 to 1000 mg of Compound A or a pharmaceutically acceptable salt form thereof in the manufacture of a medicament for treating a disorder or condition that is affected by the inhibition of MALT1.

In one embodiment, the disorder or condition is cancer and/or immunological disease. In another embodiment, the disorder or condition is lymphoma, such as, for example chronic lymphocytic leukemia (CLL) or small lymphocytic lymphoma (SLL). In yet another embodiment, disorder or condition is selected from the group consisting of diffuse large B-cell lymphoma (DLBCL), mantle cell lymphoma (MCL), follicular lymphoma (FL), and mucosa-associated lymphoid tissue (MALT) lymphoma. In another embodiment, the disorder or condition is the activated B cell like (ABC) subtype of diffuse large B-cell lymphoma (DLBCL).

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The summary, as well as the following detailed description, is further understood when read in conjunction with the appended drawings. For the purpose of illustrating the invention, there are shown in the drawings exemplary embodiments of the invention; however, the invention is not limited to the specific disclosure of the drawings.

FIG. 1A is a plot showing the assessment of synergy of Compound A and ibrutinib in HBL1 cells based on the HSA model. Red indicates data obtained with Compound A; gold indicates data obtained with ibrutinib; grey indicates the expected combination effect; black shows the actual measured combination effect; and blue is an indication of synergy.

FIG. 1B is a plot showing the assessment of synergy of Compound A and ibrutinib in OCI-Ly10 cells based on the HSA model. Red indicates data obtained with Compound A; gold indicates data obtained with ibrutinib; grey indicates the expected combination effect; black shows the actual measured combination effect; and blue is an indication of synergy.

FIG. 2 shows a visualization of point-by-point values of MaxR test statistics in the OCI-Ly10 cellular model evaluated for both Loewe and HSA null model. Blue indicates synergy, while red indicates antagonism. The bigger the size of the dots, the higher degree of synergy/antagonism while the intensity of color correlates to the statistical significance, indicated by p-values.

FIG. 3 shows a three-dimensional visualization of synergy in the OCI-Ly10 cellular model evaluated for both Loewe and HSA null model. Red dots represent data obtained with Compound A monotherapy, golden dots represent data obtained with Compound B monotherapy, grey area indicates expected combination effects, black dots represent actual data measured in combination experiment, blue area indicates statistically significant synergy.

FIG. 4 shows a visualization of point-by-point values of MaxR test statistics in the HBL1 cellular model. Blue indicates synergy, while red indicates antagonism. The bigger the size of the dots, the higher degree of synergy/antagonism while the intensity of color correlates to the statistical significance, indicated by p-values.

FIG. 5 shows a three-dimensional visualization of synergy in the HBL1 cellular model. Red dots represent data obtained with Compound A monotherapy, golden dots represent data obtained with Compound B monotherapy, grey area indicates expected combination effects, black dots represent actual data measured in combination experiment, blue area indicates statistically significant synergy.

FIG. 6 shows a visualization of point-by-point values of MaxR test statistics in the TMD8 cellular model. Blue indicates synergy, while red indicates antagonism. The bigger the size of the dots, the higher degree of synergy/antagonism while the intensity of color correlates to the statistical significance, indicated by p-values.

FIG. 7 shows a three-dimensional visualization of synergy in the TMD8 cellular model. Blue indicates synergy, while red indicates antagonism. The bigger the size of the dots, the higher degree of synergy/antagonism while the intensity of color correlates to the statistical significance, indicated by p-values.

FIG. 8 shows a visualization of point-by-point values of MaxR test statistics in the OCI-Ly3 cellular model. Blue indicates synergy, while red indicates antagonism. The bigger the size of the dots, the higher degree of synergy/antagonism while the intensity of color correlates to the statistical significance, indicated by p-values.

FIG. 9 shows a three-dimensional visualization of synergy in the OCI-Ly3 cellular model. Red dots represent data obtained with Compound A monotherapy, golden dots represent data obtained with Compound B monotherapy, grey area indicates expected combination effects, black dots represent actual data measured in combination experiment, blue area indicates statistically significant synergy.

FIG. 10 shows a visualization point-by-point values of MaxR test statistics in the REC-1 cellular model. Blue indicates synergy, while red indicates antagonism. The bigger the size of the dots, the higher degree of synergy/antagonism while the intensity of color correlates to the statistical significance, indicated by p-values.

FIG. 11 shows a three-dimensional visualization of synergy in REC-1 cellular model. Red dots represent data obtained with Compound A monotherapy, golden dots represent data obtained with Compound B monotherapy, grey area indicates expected combination effects, black dots represent actual data measured in combination experiment, blue area indicates statistically significant synergy.

FIG. 12 shows a visualization of point-by-point values of MaxR test statistics in JEKO-1 cellular model. Blue indicates synergy, while red indicates antagonism. The bigger the size of the dots, the higher degree of synergy/antagonism while the intensity of color correlates to the statistical significance, indicated by p-values.

FIG. 13 shows a visualization of point-by-point values of MaxR test statistics in the MINO cellular model Blue indicates synergy, while red indicates antagonism. The bigger the size of the dots, the higher degree of synergy/antagonism while the intensity of color correlates to the statistical significance, indicated by p-values.

FIG. 14 shows a visualization of point-by-point values of MaxR test statistics in the MAVER-1 cellular model. Blue indicates synergy, while red indicates antagonism. The bigger the size of the dots, the higher degree of synergy/antagonism while the intensity of color correlates to the statistical significance, indicated by p-values.

FIG. 15 shows the effect of Compound B on body weight of NSG mice bearing OCI-LY10 tumors in Study 1 of Example 3. SEM, standard error of the mean; PEG400/PVP-VA6, polyethylene glycol 400/1-vinyl-2-pyrrolidone and vinyl acetate 64 copolymer. Group % body weight changes are graphed as the mean±SEM. Female mice were implanted SC on the right flank on Day 0. Tumors were established 33 days post implantation, mice were randomized into experimental groups and dosed orally twice or once daily for 3 weeks (n=10/group).

FIG. 16 shows the effect of Compound B QD, Compound A BID and combination of both compounds on body weight of NSG mice bearing OCI-LY10 Tumors in Study 3 of Example 3. SEM, standard error of the mean; PEG400, polyethylene glycol 400. Group % body weight changes are graphed as the mean±SEM. Female mice were implanted SC on the right flank on Day 0. Tumors were established 35 days post implantation, mice were randomized into experimental groups and dosed once or twice daily for 3 weeks (n=10/group).

FIG. 17 shows the effect of Compound B BID, Compound A BID and combination of both compounds on body weight of mice bearing OCI-LY10 tumors in Study 4 of Example 3. SEM, standard error of the mean; PEG400, polyethylene glycol 400. Group % body weight changes are graphed as the mean±SEM. Female mice were implanted SC on the right flank on Day 0. Tumors were established 32 days post implantation and mice were randomized into experimental groups and dosed twice daily for 3 weeks (n=10/group).

FIG. 18 shows the effect of Compound B on growth of established OCI-LY10 human DLBCL xenografts in mice in Study 3 of Example 3. PEG400/PVP-VA64, Polyethylene glycol 400/1-vinyl-2-pyrrolidone and vinyl acetate 64 copolymer, SEM, standard error of the mean. Group tumor volumes are graphed as the mean±SEM. Bar below x-axis indicates the treatment period. Groups are plotted while at least ⅔ of the animals remained on the study. Mice were implanted SC on the right flank on Day 0. Tumors were established 33 days post implantation, mice were randomized into experimental groups and dosed orally twice or once daily for 3 weeks (n=10/group).

FIG. 19 shows the effect of combination treatment of Compound B QD and Compound A BID on OCI-LY10 tumor growth in mice in Study 3 of Example 3. PEG400, Polyethylene glycol 400; SEM, standard error of the mean. Group tumor volumes are graphed as the mean±SEM. Bar below x-axis indicates the treatment period. Groups are plotted while at least ⅔ of the animals remained on the study. Female mice were implanted SC on the right flank on Day 0. Tumors were established 35 days post implantation, mice were randomized into experimental groups and dosed orally QD or BID for 3 weeks (n=10/group).

FIG. 20 shows the effect of combination treatment of Compound B BID and Compound A BID on OCI-LY10 tumor growth in mice in Study 4 of Example 3. EG400, Polyethylene glycol 400; SEM, standard error of the mean. Group tumor volumes are graphed as the mean±SEM. Bar below x-axis indicates the treatment period. Groups are plotted while at least ⅔ of the animals remained on the study. Female mice were implanted SC on the right flank on Day 0. After tumors were established 32 days post implantation, mice were randomized into experimental groups and dosed orally BID for 3 weeks (n=10/group).

FIG. 21 shows circulating human IL-10 cytokine serum levels of mice treated with the BTK inhibitor Compound B. I1-10 cytokine levels are graphed as % normalized to vehicle control IL-10 levels f SEM. Female NSG mice were implanted SC on the right flank on Day 0. After tumors were established 39 days post implantation, mice were randomized into experimental groups and dosed orally with a single dose (n=5/dose level/time point). Serum samples were collected 2, 4, 8, 12, 16, and 24 hours after compound administration.

FIG. 22 shows BTK protein occupancy in OCI-LY10 DLBCL tumor lysates of NSG mice treated with the BTK inhibitor Compound B. Unoccupied BTK protein levels are graphed as % normalized to vehicle control BTK levels f SEM. Female mice were implanted SC on the right flank on Day 0. Tumors were established 39 days post implantation, randomized into experimental groups, and dosed orally with a single dose (n=5/dose level/time point). Tumor samples were harvested 4, 12, and 24 hours after compound administration.

FIG. 23 depicts tumor volume growth curves in the mouse PDX model following administration of Compound A and Compound B, either as monotherapy or as a combination.

FIG. 24 shows serum cytokine secretion levels after Day 1 following administration of Compound A and Compound B, either as monotherapy or as a combination.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

All patents, published patent applications, and publications cited herein are incorporated by reference as if set forth fully herein.

Unless defined otherwise, all technical and scientific terms used herein have the same meaning commonly understood to one of ordinary skill in the art to which this invention pertains. Otherwise, certain terms used herein have the meanings as set in the specification.

The instant disclosure relates to using the MALT1 inhibitor 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

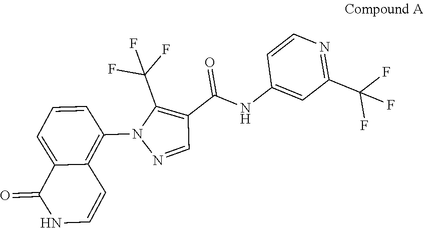 [SMILES: O=C(Nc1ccnc(C(F)(F)F)c1)c1cnn(-c2cccc3c(=O)[nH]ccc23)c1C(F)(F)F]

in combination with a BKT inhibitor to treat disorders or conditions such as cancer and/or immunological diseases. In particular, the instant disclosure is based on the surprising discovery that when Compound A is used in combination with a BKT inhibitor of Formula (I):

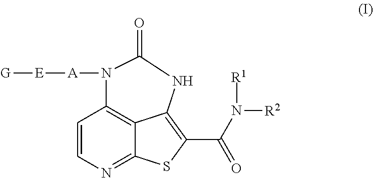

(such as N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide) to treat certain conditions, such as cancer the combined treatment provides a synergistic effect.

In certain embodiments, the instant disclosure relates to a method of treating cancer using a combination therapy using Compound A and a compound of Formula (I) (such as e.g. N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide) to treat a cancer that is sensitive to monotherapy by both compound A and the BTK inhibitor. In particular, the instant disclosure provides for a method of treating diffuse large B-cell lymphomas by administering Compound A and N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide, whereby Compound A and the carboxamide act in synergism.

### Definitions

Some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and claims of this specification, the words “comprise” and “contain” and variations of the words, for example “comprising” and “comprises”, mean “including but not limited to”, and are not intended to (and do not) exclude other components. As used herein and in the appended claims, the singular forms “a,” “an,” and “the” include plural reference unless the context clearly dictates otherwise.

The term “alkyl,” when used alone or as part of a substituent group, refers to a straight- or branched-chain alkyl group having from 1 to 12 carbon atoms (“C₁₋₁₂”), preferably 1 to 6 carbons atoms (“C₁₋₆”), in the chain. Examples of alkyl groups include methyl (Me, C₁alkyl) ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and groups that in light of the ordinary skill in the art and the teachings provided herein would be considered equivalent to any one of the foregoing examples.

The term “C₁₋₆alk” refers to an aliphatic linker having 1, 2, 3, 4, 5, or 6 carbon atoms and includes, for example, CH₂, CH(CH₃), CH(CH₃)—CH₂, and C(CH₃)₂—. The term “—C₀alk-” refers to a bond. In some aspects, the C₁₋₆alk can be substituted with an oxo group or an OH group.

The term “alkenyl,” when used alone or as part of a substituent group, refers to straight and branched carbon chains having from 2 to 12 carbon atoms (“C₂₋₁₂”), preferably 2 to 6 carbon atoms (“C₂”), wherein the carbon chain contains at least one, preferably one to two, more preferably one double bond. For example, alkenyl moieties include, but are not limited to allyl, 1-propen-3-yl, 1-buten-4-yl, propa-1,2-dien-3-yl, and the like.

The term “alkynyl,” when used alone or as part of a substituent group, refers to straight and branched carbon chains having from 2 to 12 carbon atoms (“C₂₋₁₂”), preferably 2 to 6 carbon atoms (“C₂”), wherein the carbon chain contains at least one, preferably one to two, more preferably one triple bond. For example, alkynyl moieties include, but are not limited to vinyl, 1-propyn-3-yl, 2-butyn-4-yl, and the like.

The term “aryl” refers to carbocylic aromatic groups having from 6 to 10 carbon atoms (“C₆₋₁₀”) such as phenyl, naphthyl, and the like.

The term “cycloalkyl” refers to monocyclic, non-aromatic hydrocarbon groups having from 3 to 10 carbon atoms (“C₃₋₁₀”), preferably from 3 to 6 carbon atoms (“C₃₋₆”). Examples of cycloalkyl groups include, for example, cyclopropyl (C₃), cyclobutyl (C₄), cyclopentyl (C₅), cyclohexyl (C₆), 1-methylcyclopropyl (C₄), 2-methylcyclopentyl (C₄), adamantanyl (C₁₀) and the like.

The term “heterocycloalkyl” refers to any five to ten membered monocyclic or bicyclic, saturated ring structure containing at least one heteroatom selected from the group consisting of O, N and S. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, hexahydro-5H-[1,4]dioxino[2,3-c]pyrrolyl, benzo[d][1,3]dioxolyl, and the like.

The term “heteroaryl” refers to a mono- or bicyclic aromatic ring structure including carbon atoms as well as up to four heteroatoms selected from nitrogen, oxygen, and sulfur. Heteroaryl rings can include a total of 5, 6, 9, or 10 ring atoms (“C₅₋₁₀”). Examples of heteroaryl groups include but are not limited to, pyrrolyl, furyl, thienyl, oxazolyl, imidazolyl, pyrazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

The term “haloalkyl” refers to an alkyl moiety wherein one or more of the hydrogen atoms has been replaced with one or more halogen atoms. One exemplary substitutent is fluoro. Preferred haloalkyl groups of the disclosure include trishalogenated alkyl groups such as trifluoromethyl groups.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans. Suitable pharmaceutically acceptable salts include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid, or phosphoric acid. Furthermore, where the compounds carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

The term “subject” means any animal, particularly a mammal, most particularly a human, who will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more particularly a human.

The term “therapeutically effective dose” refers to an amount of an active compound or pharmaceutical agent, including a crystalline form of the present invention, which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease.

The term “synergy” refers to an effect from combination of two (or more) drugs that is bigger than the expected additive biological activity of the individual compounds. In certain embodiments, the resulted/expected effect is dependent on the chosen null model, where common null models are HSA, Loewe, and Bliss.

Where doses of the present invention are expressed in relation to the weight of the subject, “mg/kg” is used to specify milligrams of the compound for each kilogram of the subject's body weight.

In one embodiment, the term “therapeutically effective dose” refers to the amount of Compound A or BTK inhibitor, and their respective enantiomers, diastereomers, solvates or pharmaceutically acceptable salts form thereof, that when administered to a subject, is effective to at least partially alleviate, inhibit, prevent, and/or ameliorate a condition, or a disorder or a disease.

The term “composition” refers to a product that includes the specified ingredients in therapeutically effective amounts, as well as any product that results, directly, or indirectly, from combinations of the specified ingredients in the specified amounts.

The term “administer” or “administered” or “administering” refers to the administration of Compound A or BTK inhibitor and their respective solvates or pharmaceutically acceptable salt forms thereof, or a pharmaceutical compositions thereof to a subject by any method known to those skilled in the art in view of the present disclosure, such as by intramuscular, subcutaneous, oral, intravenous, cutaneous, intramucosal (e.g., gut), intranasal or intraperitoneal route of administration. In particular embodiments, a pharmaceutical composition of the invention is administered to a subject orally.

The term “affected by the inhibition of MALT1” in the context of a disorder or disease refers to any disease, syndrome, condition, or disorder that might occur in the absence of MALT1 but can occur in the presence of MALT1. Suitable examples of a disease, syndrome, condition, or disorder that is affected by the inhibition of MALT1 include, but are not limited to, lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocytic leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharyngeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor). Additional examples include, but are not limited to, autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

As used herein, the term “condition” refers to any disease, syndrome, or disorder detected or diagnosed by a researcher, veterinarian, medical doctor, or other clinician, wherein said researcher, veterinarian, medical doctor, or other clinician determines that it desirable to seek a biological or medicinal response in an animal tissue system, particularly a mammalian or human tissue system.

As used herein, the term “disorder” refers to any disease, syndrome, or condition detected or diagnosed by a researcher, veterinarian, medical doctor, or other clinician, wherein said researcher, veterinarian, medical doctor, or other clinician determines that it desirable to seek a biological or medicinal response in an animal tissue system, particularly a mammalian or human tissue system.

As used herein, the term “MALT1 inhibitor” refers to an agent that inhibits or reduces at least one condition, symptom, disorder, and/or disease of MALT1.

As used herein, unless otherwise noted, the term “affect” or “affected” (when referring to a disease, syndrome, condition or disorder that is affected by the inhibition of MALT1) includes a reduction in the frequency and/or severity of one or more symptoms or manifestations of said disease, syndrome, condition or disorder, and/or includes the prevention of the development of one or more symptoms or manifestations of said disease, syndrome, condition or disorder or the development of the disease, condition, syndrome or disorder.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In a further embodiment, “treat,” “treating,” or “treatment” refers to modulating the disease, condition, syndrome, or disorder either physically (e.g. stabilization of a discernible symptom), physiologically, (e.g. stabilization of a physical parameter), or both. In yet another embodiment, “treat,” “treating,” or “treatment” refers to preventing or delaying the onset or development or progression of the disease, condition, syndrome, or disorder.

A skilled person will understand that references to Compound A and BTK inhibitor (including exemplified BTK inhibitors, such as for example N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide, ibrutinib, acalabrutinib, zanubrutinib) might also refer to their respective enantiomers, diastereomers, or solvates or pharmaceutically acceptable salt forms thereof, even if not explicitly referred to, and that they are also included in the scope of the present invention.

### Embodiments

### Compositions

Even though the compounds of embodiments of the present invention can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice. Thus, embodiments of the present invention are directed to pharmaceutical and veterinary compositions comprising Compound A and compositions comprising a BTK inhibitor, and at least one pharmaceutically acceptable carrier, pharmaceutically acceptable excipient, and/or pharmaceutically acceptable diluent.

By way of example, in the pharmaceutical compositions of embodiments of the present invention, Compound A and/or the BTK inhibitor may be admixed with any suitable binder(s), lubricant(s), suspending agent(s), coating agent(s), solubilizing agent(s), and combinations thereof.

Solid oral dosage forms such as, tablets or capsules, containing Compound A and/or the BTK inhibitor may be administered in at least one dosage form at a time, as appropriate. It is also possible to administer Compound A in sustained release formulations. Alternatively, Compound A and/or the BTK inhibitor may be administered as a sprinkle formulation.

Additional oral forms in which Compound A and/or the BTK inhibitor may be administered include elixirs, solutions, syrups, and suspensions; each optionally containing flavoring agents and coloring agents.

For buccal or sublingual administration, the pharmaceutical compositions of the present invention may be administered in the form of tablets or lozenges, which can be formulated in a conventional manner.

By way of further example, pharmaceutical compositions containing Compound A and/or the BTK inhibitor as the active pharmaceutical ingredient can be prepared by mixing Compound A and/or the BTK inhibitor with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, intramuscular, subcutaneous, intravenous, cutaneous, intramucosal, intranasal or intraperitoneal routes etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also may be optionally coated with substances such as, sugars, or be enterically coated so as to modulate the major site of absorption and disintegration. For parenteral administration, the carrier, excipient, and diluent will usually include sterile water, and other ingredients may be added to increase solubility and preservation of the composition. Injectable suspensions or solutions may also be prepared utilizing aqueous carriers along with appropriate additives such as, solubilizers and preservatives.

### Compound A

The term “Compound A” refers to 1-(1-oxo-1,2-dihydroisoquinolin-5-yl)-5-(trifluoromethyl)-N-[2-(trifluoromethyl)pyridin-4-yl]-1H-pyrazole-4-carboxamide having the following structure:

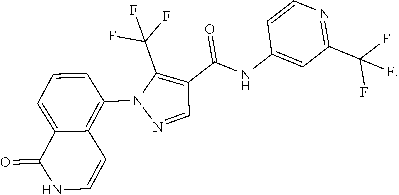 [SMILES: O=C(Nc1ccnc(C(F)(F)F)c1)c1cnn(-c2cccc3c(=O)[nH]ccc23)c1C(F)(F)F]

The invention also contemplates Compound A or an enantiomer, diastereomer, a solvate or pharmaceutically acceptable salt thereof and considers them to be within the scope of the invention.

Compound A may be prepared, for example, as described in Example 158 of WO 2018/119036, and WO 2020/169736, which are incorporated herein by reference. The procedure of Example 158 has been determined as providing Compound A hydrate.

Compound A may exist as a solvate. A “solvate” may be a solvate with water (i.e., a hydrate) or with a common organic solvent. The use of pharmaceutically acceptable solvates, said solvates including hydrates, and said hydrates including monohydrates, is considered to be within the scope of the invention.

Compound A may be formulated in an amorphous form or dissolved state; for example, and without limitation, Compound A may be formulated in an amorphous form with a polyethylene glycol (PEG) polymer.

A person of ordinary skill in the art would recognize that Compound A may exist as tautomers. It is understood that all tautomeric forms are encompassed by a structure where one possible tautomeric arrangement of the groups of the compound is described, even if not specifically indicated.

For example, it is understood that:

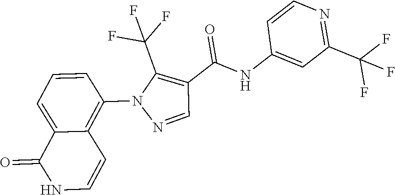 [SMILES: O=C(Nc1ccnc(C(F)(F)F)c1)c1cnn(-c2cccc3c(=O)[nH]ccc23)c1C(F)(F)F]

also encompasses the following structure:

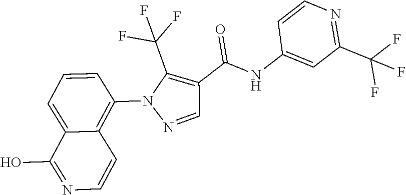 [SMILES: O=C(Nc1ccnc(C(F)(F)F)c1)c1cnn(-c2cccc3c(O)nccc23)c1C(F)(F)F]

Any convenient tautomeric arrangement may be utilized in describing the compounds.

Therapeutically effective doses of Compound A

In one embodiment of the invention, the therapeutically effective dose of Compound A is about 25 to 1000 mg. In another embodiment, the therapeutically effective dose of Compound A is about 25 to 200 mg. In yet another embodiment, the therapeutically effective dose of Compound A is about 25 to 150 mg. In an alternate embodiment, the therapeutically effective dose of Compound A is about 25 to 250 mg. In another alternate embodiment of the invention, the therapeutically effective dose of Compound A is about 25 to 350 mg.

In another embodiment of the invention, the therapeutically effective dose of Compound A is about 50 to 500 mg. In an alternate embodiment, the therapeutically effective dose of Compound A is about 50 to 200 mg. In yet another embodiment of the invention, the therapeutically effective dose of Compound A is about 50 to 150 mg.

In an embodiment of the invention, the therapeutically effective dose of Compound A is about 100 to 200 mg. In another embodiment of the invention, the therapeutically effective dose of Compound A is about 110 mg. In yet another embodiment of the invention, the therapeutically effective dose of Compound A is about 100 to 400 mg. In yet another embodiment of the invention, the therapeutically effective dose of Compound A is about 150 to 300 mg. In an alternate embodiment of the invention, the therapeutically effective dose of Compound A is about 200 mg.

In another embodiment of the invention, the therapeutically effective dose of Compound A is about 100 to 150 mg. In an additional embodiment of the invention, the therapeutically effective dose of Compound A is about 150 to 200 mg. In a further embodiment of the invention, the therapeutically effective dose of Compound A is about 200 to 250 mg. In yet another embodiment of the invention, the therapeutically effective dose of Compound A is about 250 to 300 mg. In an alternate embodiment of the invention, the therapeutically effective dose of Compound A is about 300 to 350 mg. In yet another embodiment of the invention, the therapeutically effective dose of Compound A is about 350 to 400 mg.

In another embodiment of the invention, the therapeutically effective dose is an amount sufficient to maintain a plasma level of Compound A from about 4,500 ng/mL to about 4,750 ng/mL. In an alternate embodiment of the invention, the therapeutically effective dose is an amount sufficient to maintain a plasma level of Compound A of about 4,640 ng/ml. In yet another embodiment, the therapeutically effective dose is an amount sufficient to maintain a plasma level of Compound A of about 4,550 to 4,700 ng/ml. In another embodiment, the therapeutically effective dose is an amount sufficient to maintain a plasma level of Compound A of about 4,600 to 4,700 ng/ml. In an alternate embodiment, the therapeutically effective dose is an amount sufficient to maintain a plasma level of Compound A of about 4,550 to 4,680 ng/ml.

In another embodiment of the invention, the therapeutically effective dose of Compound A is administered twice (two times) a day. In an alternate embodiment of the invention, the therapeutically effective dose of Compound A is administered one time a day. In some embodiments, the therapeutically effective dose of Compound A is administered twice daily for 7 days (loading dose), followed by once daily administration.

In another embodiment of the invention, the therapeutically effective dose of Compound A is administered on a continuous 28-day cycle. In an alternate embodiment of the invention, the therapeutically effective dose of Compound A is administered on a continuous 21-day cycle.

### BTK Inhibitors

Various BTK inhibitors may be used in combination with compound A. The BTK inhibitor may be used in combination with compound A using any of the therapeutically effective dose, administration interval and dosage cycle for compound A. The BTK inhibitor may be used in combination with compound A to treat any of the disease or conditions described herein.

In certain embodiments, the BTK inhibitor and compound A may be used to treat cancer. In particular, the BTK inhibitor and compound A may be used to treat activated B cell like (ABC) subtype of diffuse large B-cell lymphoma (DLBCL).

In one embodiment, the BTK inhibitor is ibrutinib (1-[(3R)-3-[4-amino-3-(4-phenoxyphenyl)pyrazolo[3,4-d]pyrimidin-1-yl]piperidin-1-yl]prop-2-en-1-one). In another embodiment, the BTK inhibitor is Roche BTKi RN486. In yet another embodiment, the BTK inhibitor is acalabrutinib (4-[8-amino-3-[(2S)-1-but-2-ynoylpyrrolidin-2-yl]imidazo[1,5-a]pyrazin-1-yl]-N-pyridin-2-ylbenzamide). In yet another embodiment, the BTK inhibitor is zanubrutinib (S)-7-(1-acryloylpiperidin-4-yl)-2-(4 phenoxyphenyl)-4,5,6,7-tetrahydropyrazolo[1,5-a]pyrimidine-3-carboxamide. Other BTK inhibitors that may be used in combination with Compound A are CT-1530, DTRMWXHS-12, spebrutinib besylate, vecabrutinib, evobrutinib, tirabrutinib, fenebrutinib, poseltinib, BMS-986142, ARQ-531, LOU-064, PRN-1008, ABBV-599, AC-058, BIIB-068, BMS-986195, HWH-486, PRN-2246, TAK-020, GDC-0834, BMX-IN-1, RN486, SNS-062, LFM-A13, and PCI-32765.

In another embodiment, the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide (Compound B).

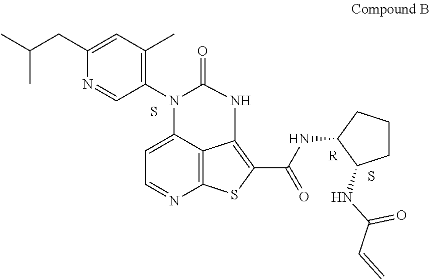

N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide may be prepared, for example, as described in Example 298 of WO 2018/103060, WO 2017/100662, US 2017/0283430, and US 2019/0276471, which are incorporated herein by reference.

In alternate embodiments of combination therapy, the BTK inhibitor is a compound of Formula (I):

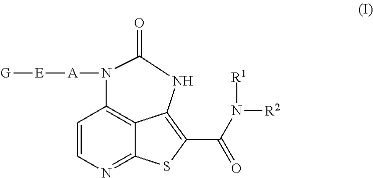

wherein

R¹ is H or C₁₋₆alkyl;

R² is selected from the group consisting of: C₀₋₆alk-cycloalkyl optionally substituted with 1, 2, or 3 substituents each independently selected from the group consisting of: NR⁸—C(O)—C(R³)═CR⁴(R⁵); NR⁶R⁷; OH; CN; oxo; O—C₁₋₆alkyl; halogen; C₁₋₆alkyl; C₁₋₆haloalkyl; C₁₋₆alk-OH; C₁₋₆cycloalkyl; C₁₋₆alkaryl; SO₂C₁₋₆alkyl; SO₂C₂₋₆alkenyl; NR⁸—C(O)—C₁₋₆alk-NR⁶R⁷; NR⁸—C(O)—C₁₋₆alkyl; NR⁸—C(O)—O—C₁₋₆alkyl; NR⁸—C(O)—C₃₋₆cycloalkyl; NR⁸—C(O)H; NR⁸—C(O)—C₃₋₆cycloalkyl; NR⁸—C(O)—C₁₋₆haloalkyl; NR⁸—C(O)-alkynyl; NR⁸—C(O)—C₆₋₁₀aryl; NR⁸—C(O)-heteroaryl; NR⁸—C(O)—C₁₋₆alk-CN; NR⁸—C(O)—C₁₋₆alk-OH; NR⁸—C(O)—C₁₋₆alk-SO₂—C₁₋₆alkyl; NR⁸—C(O)—C₁₋₆alk-NR⁶R⁷; NR⁸—C(O)—C₁₋₆alk-O—C₁₋₆alkyl wherein the C₁₋₆alk is optionally substituted with OH, OC₁₋₆alkyl, or NR⁶R⁷; and NR⁸—C(O)—C₀₋₆alk-heterocycloalkyl wherein the C₀₋₆alk is optionally substituted with oxo and the heterocycloalkyl is optionally substituted with C₁₋₆alkyl;

wherein R⁶ and R⁷ are each independently selected from the group consisting of: H; C₁₋₆alkyl; C₃₋₆cycloalkyl; C(O)H; and CN;

R³ is selected from the group consisting of: H, CN, halogen, C₁₋₆haloalkyl, and C₁₋₆alkyl;

R⁴ and R⁵ are each independently selected from the group consisting of: H; C₀₋₆alk-NR⁶R⁷; C₁₋₆alk-OH; C₀₋₆alk-C₃₋₆cycloalkyl optionally substituted with C₁₋₆alkyl; halogen; C₁₋₆alkyl; OC₁₋₆alkyl; C₁₋₆alk-O—C₁₋₆alkyl; C₁₋₆alk-NH—C₀₋₆alk-O—C₁₋₆alkyl; C₀₋₆alk-heterocycloalkyl optionally substituted with C(O)C₁₋₆alkyl or C₁₋₆alkyl; C₁₋₆alk-NHSO₂—C₁₋₆alkyl; C₁₋₆alk-SO₂—C₁₋₆alkyl; —NHC(O)—C₁₋₆alkyl; and -linker-PEG-Biotin;

R⁸ is H or C₁₋₆alkyl;

or R¹ and R², together with the nitrogen atom to which they are attached, form a pyrrolidinyl ring optionally substituted with NR⁶R⁷, wherein R⁶ and R⁷ are each independently selected from the group consisting of H; C₁₋₆alkyl; NR⁸—C(O)—C₁₋₆alkyl; and NR⁸—C(O)—C(R³)═CR⁴(R⁵), wherein R⁸ is H; R³ is H or CN; R⁴ is H; and R⁵ is H or cyclopropyl;

A is selected from the group consisting of: a bond; pyridyl; phenyl; napthalenyl; pyrimidinyl; pyrazinyl; pyridazinyl; benzo[d][1,3]dioxolyl optionally substituted with halogen; benzothiophenyl; and pyrazolyl; wherein the A is optionally substituted with 1, 2, or 3 substituents each independently selected from the group consisting of: C₁₋₆alkyl; halogen; SF₅; OC₁₋₆alkyl; C(O)—C₁₋₆alkyl; and C₁₋₆ haloalkyl;

E is selected from the group consisting of: O, a bond, C(O)—NH, CH₂, and CH₂—O;

G is selected from the group consisting of: H; C₃₋₆cycloalkyl; phenyl; thiophenyl; C₁₋₆alkyl; pyrimidinyl; pyridyl; pyridazinyl; benzofuranyl; C₁₋₆haloalkyl; heterocycloalkyl that contains an oxygen heteroatom; phenyl-CH₂—O-phenyl; C₁₋₆alk-O—C₁₋₆alkyl; NR⁶R⁷; SO₂C₁₋₆alkyl; and OH; wherein the phenyl; pyridyl; pyridazinyl; benzofuranyl; or thiophenyl is optionally substituted with 1, 2, or 3 substituents each independently selected from the group consisting of: halogen; C₁₋₆alkyl; C₁₋₆haloalkyl; OC₁₋₆haloalkyl; C₃₋₆cycloalkyl; OC₁₋₆alkyl; CN; OH; C₁₋₆alk-O—C₁₋₆alkyl; C(O)—NR⁶R⁷; and C(O)—C₁₋₆alkyl; and

stereoisomers, solvates, and isotopic variants thereof; and pharmaceutically acceptable salts thereof.

These BTK inhibitors are disclosed in WO 2018/103060, WO 2017/100662, US 2017/0283430, and US 2019/0276471, the disclosures of each of which as they pertain to BTK inhibitors and their synthesis are incorporated herein.

### Therapeutically Effective Doses of BTK Inhibitor

Effective amounts or doses of the BTK inhibitors of the present disclosure may be ascertained by routine methods such as modelling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of the BTK inhibitor per kg of subject's body weight per day, alternatively from about 0.005 to 150 mg of the BTK inhibitor per kg of subject's body weight per day, alternatively from about 0.05 to 150 mg/kg/day, alternatively from about 0.05 to about 125 mg/kg/day, alternatively from about 1 to about 50 mg/kg/day, alternatively about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

In one embodiment of the invention, the therapeutically effective dose of BTK inhibitor is about 25 to 1000 mg. In another embodiment, the therapeutically effective dose of BTK inhibitor is about 25 to 200 mg. In yet another embodiment, the therapeutically effective dose of BTK inhibitor is about 25 to 150 mg. In an alternate embodiment, the therapeutically effective dose of BTK inhibitor is about 25 to 250 mg. In another alternate embodiment of the invention, the therapeutically effective dose of BTK inhibitor is about 25 to 350 mg.

In another embodiment of the invention, the therapeutically effective dose of BTK inhibitor is about 50 to 500 mg. In an alternate embodiment, the therapeutically effective dose of BTK inhibitor is about 50 to 200 mg. In yet another embodiment of the invention, the therapeutically effective dose of BTK inhibitor is about 50 to 150 mg.

In an embodiment of the invention, the therapeutically effective dose of BTK inhibitor is about 100 to 200 mg. In another embodiment of the invention, the therapeutically effective dose of BTK inhibitor is about 110 mg. In yet another embodiment of the invention, the therapeutically effective dose of BTK inhibitor is about 100 to 400 mg. In yet another embodiment of the invention, the therapeutically effective dose of BTK inhibitor is about 150 to 300 mg. In an alternate embodiment of the invention, the therapeutically effective dose of BTK inhibitor is about 200 mg.

In another embodiment of the invention, the therapeutically effective dose of BTK inhibitor is about 100 to 150 mg. In an additional embodiment of the invention, the therapeutically effective dose of BTK inhibitor is about 150 to 200 mg. In a further embodiment of the invention, the therapeutically effective dose of BTK inhibitor is about 200 to 250 mg. In yet another embodiment of the invention, the therapeutically effective dose of BTK inhibitor is about 250 to 300 mg. In an alternate embodiment of the invention, the therapeutically effective dose of BTK inhibitor is about 300 to 350 mg. In yet another embodiment of the invention, the therapeutically effective dose of BTK inhibitor is about 350 to 400 mg.

### Disorder or Condition

The disorder or condition may be a cancer and/or immunological diseases.

In one embodiment, the disorder or condition is selected from cancers of hematopoietic origin or solid tumors such as chronic myelogenous leukemia, myeloid leukemia, non-Hodgkin lymphoma, and other B cell lymphomas.

In another embodiment, the disorder or condition includes, but is not limited to cancers, such as lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

In another embodiment, the disorder or condition is selected from diffuse large B-cell lymphoma (DLBCL), mantle cell lymphoma (MCL), follicular lymphoma (FL), and mucosa-associated lymphoid tissue (MALT) lymphoma.

In another embodiment of the invention, the disorder or condition is lymphoma.

In another embodiment of the invention, the disorder or condition is the activated B cell like (ABC) subtype of diffuse large B-cell lymphoma (DLBCL).

In another embodiment of the invention, the disorder or condition is chronic lymphocytic leukemia (CLL).

In another embodiment of the invention, the disorder or condition small lymphocytic lymphoma (SLL).

In another embodiment of the invention, the subjects have received prior treatment with a Bruton tyrosine kinase inhibitor (BTKi).

In another embodiment of the invention, the lymphoma is MALT lymphoma.

In another embodiment of the invention, the disorder or condition is Waldenström macroglobulinemia (WM).

In another embodiment of the invention, the disorder or condition is relapsed or refractory to prior treatment.

In another embodiment of the invention, the subject is relapsed or refractory to prior treatment with a Bruton tyrosine kinase inhibitor (BTKi).

In certain embodiments, the disorder or condition that is affected by the inhibition of MALT1 is also affected by the inhibition of BTK.

In another embodiment, the disorder or condition is an immunological disease, syndrome, disorder, or condition selected from the group consisting of rheumatoid arthritis (RA), psoriatic arthritis (PsA), autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

In another embodiment, the disorder or condition is selected from the group consisting of diffuse large B-cell lymphoma (DLBCL), mantle cell lymphoma (MCL), follicular lymphoma (FL), and mucosa-associated lymphoid tissue (MALT) lymphoma rheumatoid arthritis (RA), psoriatic arthritis (PsA), psoriasis (Pso), ulcerative colitis (UC), Crohn's disease, systemic lupus erythematosus (SLE), asthma, and chronic obstructive pulmonary disease (COPD).

In another embodiment, the disorder or condition is selected from the group consisting of diffuse large B-cell lymphoma (DLBCL), mantle cell lymphoma (MCL), follicular lymphoma (FL), mucosa-associated lymphoid tissue (MALT) lymphoma, marginal zone lymphoma, chronic lymphocytic leukemia (CLL), small lymphocytic lymphoma (SLL), and Waldenström macroglobulinemia.

In another embodiment, the disorder or condition is non-Hodgkin's lymphoma (NHL). In a further embodiment, the non-Hodgkin's lymphoma (NHL) is B-cell NHL.

### Methods of Treatment

One aspect of the invention is directed to methods of treating a disorder or condition that is affected by the inhibition of MALT1 in a subject in need of treatment, comprising administering a therapeutically effective dose of a BTK inhibitor and a therapeutically effective dose of Compound A. In some embodiments, the invention is directed to methods of treating a cancer or an immunological disease disclosed herein in a subject in need of treatment, comprising administering a therapeutically effective dose of a BTK inhibitor and a therapeutically effective dose of Compound A. In some embodiments, the combination of Compound A and BTK inhibitor has a synergistic effect in treating cancer or immunological disease in a subject.

A skilled person will understand that references to Compound A and BTK inhibitor in the section “Methods of Treatment”, might also refer to respective enantiomers, diastereoisomers, solvates or pharmaceutically acceptable salts form thereof, even if not explicitly referred to, and that they are also included in the scope of the present invention.

A therapeutically effective amount of Compound A or BTK inhibitor includes a dose range from about 100 mg to about 1000 mg, or any particular amount or range therein, in particular, from about 100 mg to about 400 mg, or any particular amount or range therein, of active pharmaceutical ingredient in a regimen of about 1 to about (4×) per day for an average (70 kg) human.

In an alternate embodiment, a therapeutically effective amount of Compound A or a BTK inhibitor includes a dose range from about 25 mg to about 1000 mg, or any particular amount or range therein, in particular, from about 25 mg to about 400 mg, or any particular amount or range therein, of active pharmaceutical ingredient in a regimen of about 1 to about (4×) per day for an average (70 kg) human.

Compound A or BTK inhibitor may be administered in a single daily dose, or the total daily dosage may be administered in divided doses of two, three and 4× daily.

In one embodiment, the invention comprises a method of treating a disorder or condition that is affected by the inhibition of MALT1 in a subject in need of treatment, comprising administering a therapeutically effective dose ranging from about 25 to 1000 mg, alternatively about 25 to 750 mg, alternatively about 25 to 500 mg, alternatively about 50 to 1000 mg, alternatively about 100 to 1000 mg of a BTK inhibitor or pharmaceutically acceptable salt form thereof, and a therapeutically effective dose ranging from about 25 to 1000 mg, alternatively about 25 to 750 mg, alternatively about 25 to 500 mg, alternatively about 50 to 1000 mg, alternatively about 100 to 1000 mg of Compound A or a pharmaceutically acceptable salt form thereof to said subject. In certain embodiments, the method comprises providing a composition containing Compound A and the BTK inhibitor. In some embodiments, the method comprises providing Compound A and BKT inhibitor in different compositions.

In one embodiment, the invention comprises Compound A or a pharmaceutically acceptable salt form thereof and a BTK inhibitor or a pharmaceutically acceptable salt form thereof, for use in treating a disorder or condition that is affected by the inhibition of MALT1 in a subject, by administration to said subject Compound A and BTK inhibitor each in an amount of from about 25 to 1000 mg, alternatively about 50 to 1000 mg, alternatively about 100 to 1000 mg.

In one embodiment, the invention comprises Compound A or a pharmaceutically acceptable salt form thereof and a BTK inhibitor or a pharmaceutically acceptable salt form thereof, for use in treating a disorder or condition that is affected by the inhibition of MALT1 in a subject, by administration to said subject a therapeutically effective dose ranging from about 25 to 1000 mg, alternatively about 25 to 750 mg, alternatively about 25 to 500 mg, alternatively about 50 to 1000 mg, alternatively about 100 to 1000 mg of a BTK inhibitor or pharmaceutically acceptable salt form thereof, and a therapeutically effective dose ranging from about 25 to 1000 mg, alternatively about 25 to 750 mg, alternatively about 25 to 500 mg, alternatively about 50 to 1000 mg, alternatively about 100 to 1000 mg of Compound A or a pharmaceutically acceptable salt form thereof to said subject. In certain embodiments, the invention comprises providing a composition containing Compound A and the BTK inhibitor. In some embodiments, the invention comprises providing Compound A and BKT inhibitor in different compositions.

In one embodiment, the invention comprises a therapeutically effective dose ranging from about 25 to 1000 mg, alternatively about 25 to 750 mg, alternatively about 25 to 500 mg, alternatively about 50 to 1000 mg, alternatively about 100 to 1000 mg of a BTK inhibitor or a pharmaceutically acceptable salt form thereof, and a therapeutically effective dose ranging from about 25 to 1000 mg, alternatively about 25 to 750 mg, alternatively about 25 to 500 mg, alternatively about 50 to 1000 mg, alternatively about 100 to 1000 mg of Compound A or a pharmaceutically acceptable salt form thereof, for use in treating a disorder or condition that is affected by the inhibition of MALT1. In certain embodiments, the invention comprises providing a composition containing Compound A and the BTK inhibitor. In some embodiments, the invention comprises providing Compound A and BKT inhibitor in different compositions.

In one embodiment, the invention comprises a BTK inhibitor or a pharmaceutically acceptable salt form thereof and Compound A or a pharmaceutically acceptable salt form thereof, for use in treating a disorder or condition that is affected by the inhibition of MALT1, wherein the BTK inhibitor or a pharmaceutically acceptable salt form thereof is administered in a therapeutically effective dose ranging from about 25 to 1000 mg, alternatively about 25 to 750 mg, alternatively about 25 to 500 mg, alternatively about 50 to 1000 mg, alternatively about 100 to 1000 mg, and wherein Compound A or pharmaceutically acceptable salt form thereof is administered in a therapeutically effective dose ranging from about 25 to 1000 mg, alternatively about 25 to 750 mg, alternatively about 25 to 500 mg, alternatively about 50 to 1000 mg, alternatively about 100 to 1000 mg. In certain embodiments, the invention comprises providing a composition containing Compound A and the BTK inhibitor. In some embodiments, the invention comprises providing Compound A and BKT inhibitor in different compositions.

In one embodiment, the invention comprises Compound A or a pharmaceutically acceptable salt form thereof and a BTK inhibitor or a pharmaceutically acceptable salt form thereof, for use in a method of treating a disorder or condition that is affected by the inhibition of MALT1 in a subject, wherein the method comprises administration to said subject Compound A and BTK inhibitor each in an amount of from about 25 to 1000 mg, alternatively from about 50 to 1000 mg, alternatively about 100 to 1000 mg.

In one embodiment, the invention comprises Compound A or a pharmaceutically acceptable salt form thereof and a BTK inhibitor or a pharmaceutically acceptable salt form thereof, for use in a method of treating a disorder or condition that is affected by the inhibition of MALT1 in a subject, by administration to said subject a therapeutically effective dose ranging from about 25 to 1000 mg, alternatively about 25 to 750 mg, alternatively about 25 to 500 mg, alternatively about 50 to 1000 mg, alternatively about 100 to 1000 mg of a BTK inhibitor or pharmaceutically acceptable salt form thereof, and a therapeutically effective dose ranging from about 25 to 1000 mg, alternatively about 25 to 750 mg, alternatively about 25 to 500 mg, alternatively about 50 to 1000 mg, alternatively about 100 to 1000 mg of Compound A or a pharmaceutically acceptable salt form thereof to said subject. In certain embodiments, the invention comprises providing a composition containing Compound A and the BTK inhibitor. In some embodiments, the invention comprises providing Compound A and BKT inhibitor in different compositions.

In one embodiment, the invention comprises Compound A or pharmaceutically acceptable salt form thereof and a BTK inhibitor or a pharmaceutically acceptable salt form thereof, for use in treating a disorder or condition that is affected by the inhibition of MALT1 in a subject, wherein Compound A is 1-(1-oxo-1,2-dihydroisoquinolin-5-yl)-5-(trifluoromethyl)-N-[2-(trifluoromethyl)pyridin-4-yl]-1H-pyrazole-4-carboxamide:

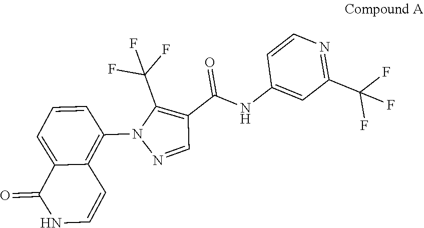 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof, and is administered to said subject in an amount of from about 25 to 1000 mg, alternatively from about 50 to 1000 mg, alternatively about 100 to 1000 mg, and BTK inhibitor or a pharmaceutically acceptable salt form thereof is administered to said subject in an amount of from about 25 to 1000 mg, alternatively from about 50 to 1000 mg, alternatively about 100 to 1000 mg.

In one embodiment, the invention comprises a method of treating cancer or an immunological disease in a subject in need of treatment, comprising administering a therapeutically effective dose ranging from about 25 to 1000 mg, alternatively from about 50 to 1000 mg, alternatively about 100 to 1000 mg of a BTK inhibitor or pharmaceutically acceptable salt form thereof and a therapeutically effective dose ranging from about 25 to 1000 mg, alternatively from about 50 to 1000 mg, alternatively about 100 to 1000 mg of Compound A or a hydrate or a pharmaceutically acceptable salt form thereof to said subject.

In one embodiment, the invention comprises Compound A or a hydrate or pharmaceutically acceptable salt form thereof and a BTK inhibitor or pharmaceutically acceptable salt form thereof, for use in treating cancer or an immunological disease in a subject, by administration to said subject Compound A and BTK inhibitor each in an amount of from about 25 to 1000 mg, alternatively from about 50 to 1000 mg, alternatively about 100 to 1000 mg.

In one embodiment, the invention comprises Compound A or a hydrate or pharmaceutically acceptable salt form thereof and a BTK inhibitor or pharmaceutically acceptable salt form thereof, for use in a method of treating a cancer or an immunological disorder in a subject, wherein the method comprises administration to said subject Compound A and BTK inhibitor each in an amount of from about 25 to 1000 mg, alternatively from about 50 to 1000 mg, alternatively about 100 to 1000 mg.

In one embodiment, the invention comprises Compound A, or a hydrate or pharmaceutically acceptable salt form thereof and a BTK inhibitor pharmaceutically acceptable salt form thereof, for use in treating cancer or an immunological disease in a subject, wherein Compound A is 1-(1-oxo-1,2-dihydroisoquinolin-5-yl)-5-(trifluoromethyl)-N-[2-(trifluoromethyl)pyridin-4-yl]-1H-pyrazole-4-carboxamide:

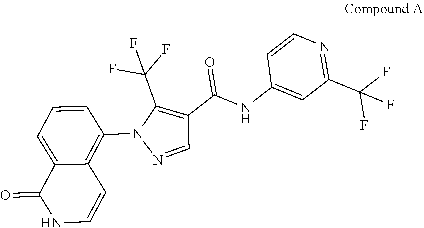 [SMILES: O=C(Nc1ccnc(C(F)(F)F)c1)c1cnn(-c2cccc3c(=O)[nH]ccc23)c1C(F)(F)F]

or a hydrate or pharmaceutically acceptable salt form thereof, is administered to said subject in an amount of from about 25 to 1000 mg, alternatively from about 50 to 1000 mg, alternatively about 100 to 1000 mg, and a BTK inhibitor or a pharmaceutically acceptable salt form thereof is administered to said subject in an amount of from about 25 to 1000 mg, alternatively from about 50 to 1000 mg, alternatively about 100 to 1000 mg.

In another embodiment of the invention, the subject is a human.

In another embodiment of the invention, Compound A is used as a hydrate form thereof. In another embodiment of the invention, Compound A is used as a monohydrate form thereof. In yet an alternate embodiment of the invention, the subject is administered a pharmaceutical composition of Compound A or a solvate or pharmaceutically acceptable salt form thereof comprising a pharmaceutically acceptable carrier, a pharmaceutically acceptable excipient, and/or a pharmaceutically acceptable diluent.

### Specific Embodiments of Combination of Compound a and BTK Inhibitors

In certain embodiments, provided herein are pharmaceutical compositions comprising Compound A or a pharmaceutically acceptable form thereof, in combination with BTK inhibitor or a pharmaceutically acceptable form thereof. In certain embodiments, the combination is in a therapeutically effective amount. In certain embodiments, the combination is in a synergistically therapeutically effective amount. In certain embodiments, the combination is synergistic. In certain embodiments, the combination has a synergistic effect. In certain embodiments, the combination has a synergistic anti-cancer effect. In certain embodiments, the combination has a synergistic therapeutic effect.

In certain embodiments, Compound A may be formulated in a composition comprising Compound A and a BTK inhibitor. In some embodiments, Compound A and BTK inhibitor are in different compositions. In one embodiment of the composition, the BTK inhibitor is ibrutinib (1-[(3R)-3-[4-amino-3-(4-phenoxyphenyl)pyrazolo[3,4-d]pyrimidin-1-yl]piperidin-1-yl]prop-2-en-1-one). In another embodiment of the composition, the BTK inhibitor is Roche BTKi RN486. In yet another embodiment, the BTK inhibitor is acalabrutinib (benzamide, 4-[8-amino-3-[(2S)-1-(1-oxo-2-butyn-1-yl)-2-pyrrolidinyl]imidazo[1,5-a]pyrazin-1-yl]-N-2-pyridinyl-). In yet another embodiment, the BTK inhibitor is zanubrutinib (S)-7-(1-acryloylpiperidin-4-yl)-2-(4 phenoxyphenyl)-4,5,6,7-tetrahydropyrazolo[1,5-a]pyrimidine-3-carboxamide. In another embodiment, the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide (Compound B).

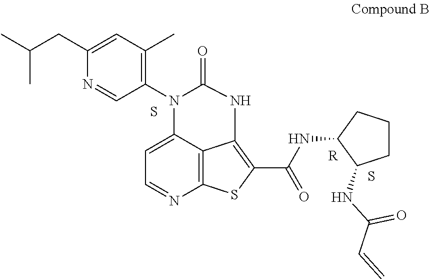

In other embodiments, any of the combinations of therapeutically effective dose, administration interval and dosage cycle shown in the Table 1 below may be used:

TABLE 1

| Therapeutically | Therapeutically | | |
|---|---|---|---|
| effective dose of | effective dose of | Administration | |
| Compound A | BTK inhibitor | interval | Dosage Cycle |
| about 25 to about 500 | about 25 to about 500 | one time a day | continuous 7- |
| mg, alternatively about | mg, alternatively about | | day cycle |
| 50 to about 500 mg; | 50 to about 500 mg; | | |
| alternatively about 100 | alternatively about 100 | | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, | | |
| 100 to 150 mg; | about 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg | to 400 mg | | |
| about 25 to about 500 | about 25 to about 500 | one time a day | continuous 21- |
| mg, alternatively about | mg, alternatively about | | day cycle |
| 50 to about 500 mg; | 50 to about 500 mg; | | |
| alternatively about 100 | alternatively about 100 | | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, | | |
| 100 to 150 mg; | about 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg | to 400 mg | | |
| about 25 to about 500 | about 25 to about 500 | one time a day | continuous 28- |
| mg, alternatively about | mg, alternatively about | | day cycle |
| 50 to about 500 mg; | 50 to about 500 mg; | | |
| alternatively about 100 | alternatively about 100 | | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, about | | |
| 100 to 150 mg; | 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg | to 400 mg | | |
| about 25 to about 500 | about 25 to about 500 | twice (two times) | continuous 7- |
| mg, alternatively about | mg, alternatively about | a day | day cycle |
| 50 to about 500 mg; | 50 to about 500 mg; | | |
| alternatively about 100 | alternatively about 100 | | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, | | |
| 100 to 150 mg; | about 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg | to 400 mg | | |
| about 25 to about 500 | about 25 to about 500 | twice (two times) | continuous 21- |
| mg, alternatively about | mg, alternatively about | a day | day cycle |
| 50 to about 500 mg; | 50 to about 500 mg; | | |
| alternatively about 100 | alternatively about 100 | | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, | | |
| 100 to 150 mg; | about 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg; | to 400 mg; | | |
| about 25 to about 500 | about 25 to about 500 | twice (two times) | continuous 28- |
| mg, alternatively about | mg, alternatively about | a day | day cycle |
| 50 to about 500 mg; | 50 to about 500 mg; | | |
| alternatively about 100 | alternatively about 100 | | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, | | |
| 100 to 150 mg; | about 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg | to 400 mg | | |
| about 25 to about 500 | about 25 to about 500 | Compound A | continuous 7- |
| mg, alternatively about | mg, alternatively about | once daily and | day cycle |
| 50 to about 500 mg; | 50 to about 500 mg; | BTK inhibitor | |
| alternatively about 100 | alternatively about 100 | twice daily | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, | | |
| 100 to 150 mg; | about 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg | to 400 mg | | |
| about 25 to about 500 | about 25 to about 500 | Compound A | continuous 21- |
| mg, alternatively about | mg, alternatively about | once daily and | day cycle |
| 50 to about 500 mg; | 50 to about 500 mg; | BTK inhibitor | |
| alternatively about 100 | alternatively about 100 | twice daily | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, | | |
| 100 to 150 mg; | about 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg | to 400 mg | | |
| about 25 to about 500 | about 25 to about 500 | Compound A | continuous 21- |
| mg, alternatively about | mg, alternatively about | once daily and | day cycle |
| 50 to about 500 mg; | 50 to about 500 mg; | BTK inhibitor | |
| alternatively about 100 | alternatively about 100 | twice daily | |
| to 400 mg; | to 400 mg; | | |
| alternatively, about 150 | alternatively, about 150 | | |
| to 300 mg; | to 300 mg; | | |
| alternatively, about 200 | alternatively, about 200 | | |
| mg; alternatively, about | mg; alternatively, | | |
| 100 to 150 mg; | about 100 to 150 mg; | | |
| alternatively, about 150 | alternatively, about 150 | | |
| to 200 mg; | to 200 mg; | | |
| alternatively, about 200 | alternatively, about 200 | | |
| to 250 mg; | to 250 mg; | | |
| alternatively, about 250 | alternatively, about 250 | | |
| to 300 mg; | to 300 mg; | | |
| alternatively, about 300 | alternatively, about 300 | | |
| to 350 mg; | to 350 mg; | | |
| alternatively, about 350 | alternatively, about 350 | | |
| to 400 mg | to 400 mg | | |
| about 100 mg, about 200 | about 140 mg, about 210 | Compound A | |
| mg or about 300 mg | mg, about 280 mg, | twice daily for 7 | |
| | about 420 mg, or about | days followed by | |
| | 560 mg | once daily; and | |
| | | BTK inhibitor | |
| | | twice daily | |
| about 100 mg, about | about 140 mg, about | Compound A | |
| 200 mg or about 300 | 210 mg, about 280 mg, | twice daily for 7 | |
| mg | about 420 mg, or about | days followed by | |
| | 560 mg | once daily; and | |
| | | BTK inhibitor | |
| | | once daily | |
| about 100 mg, about | about 140 mg, about | Compound A | |
| 200 mg or about 300 | 210 mg, about 280 mg, | once daily; and | |
| mg | about 420 mg, or about | BTK inhibitor | |
| | 560 mg | twice daily | |
| about 100 mg, about | about 140 mg, about | Compound A | |
| 200 mg or about 300 | 210 mg, about 280 mg, | once daily; and | |
| mg | about 420 mg, or about | BTK inhibitor | |
| | 560 mg | once daily | |

Another embodiment of the invention is a therapeutically effective dose of Compound A and BTK inhibitor, each ranging from about 25 to about 1000 mg, alternatively about 100 to 1000 mg, alternatively from about 100 to 400 mg alternatively from about 150 to 300 mg, alternatively about 200 mg, alternatively from about 100 to 150 mg, alternatively from about 150 to 200 mg, alternatively from about 200 to 250 mg, alternatively from about 250 to 300 mg, alternatively from about 300 to 350 mg, alternatively from about 350 to 400 mg for use in treating a disorder or condition that is affected by the inhibition of MALT1.

Yet another embodiment of the invention is use of a therapeutically effective dose of Compound A and BTK inhibitor, each ranging from about 25 to about 1000 mg, alternatively about 100 to 1000 mg, alternatively from about 100 to 400 mg alternatively from about 150 to 300 mg, alternatively about 200 mg, alternatively from about 100 to 150 mg, alternatively from about 150 to 200 mg, alternatively from about 200 to 250 mg, alternatively from about 250 to 300 mg, alternatively from about 300 to 350 mg, alternatively from about 350 to 400 mg for treating a disorder or condition that is affected by the inhibition of MALT1.

An alternate embodiment of the invention is use of a therapeutically effective dose of Compound A and BTK inhibitor, each ranging from about 25 to about 1000 mg, alternatively from about 100 to 1000 mg, alternatively from about 100 to 400 mg alternatively from about 150 to 300 mg, alternatively about 200 mg, alternatively from about 100 to 150 mg, alternatively from about 150 to 200 mg, alternatively from about 200 to 250 mg, alternatively from about 250 to 300 mg, alternatively from about 300 to 350 mg, alternatively from about 350 to 400 mg in the manufacture of a medicament for treating a disorder or condition that is affected by the inhibition of MALT1.

An alternate embodiment of the invention is use of a therapeutically effective dose of Compound A and Ibrutinib, each ranging from about 25 to about 1000 mg, alternatively from about 25 to 500 mg, alternatively from about 25 to 250 mg, alternatively from about 25 to 400 mg alternatively from about 25 to 300 mg, alternatively from about 25 to 150 mg, alternatively from about 25 to 200 mg, alternatively from about 25 to 300 mg, alternatively from about 25 to 350 mg, alternatively from about 35 to 400 mg, alternatively from about 35 to about 500 mg in the manufacture of a medicament for treating a disorder or condition that is affected by the inhibition of MALT1.

Accordingly, one embodiment of the invention is a method of treating a disorder or condition that is affected by the inhibition of MALT1 in a subject in need of treatment, comprising administering a composition comprising a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of BTK inhibitor and a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of Compound A. In certain embodiments, the disorder or condition is sensitive to treatment by both the BTK inhibitor and Compound A.

In another embodiment of the invention, the method of treating a disorder or condition that is affected by the inhibition of MALT1 in a subject in need of treatment comprises: a step of administering a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of Compound A; and a step of administering a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of BTK inhibitor. The BTK inhibitor may be administered before Compound A, after Compound A or concurrently with Compound A.

Another embodiment of the invention is a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of BTK inhibitor or pharmaceutically acceptable salt form thereof, and a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of Compound A or a pharmaceutically acceptable salt form thereof for use in treating a disorder or condition that is affected by the inhibition of MALT1. In addition, embodiments of the invention are directed to use of a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of BTK inhibitor or a pharmaceutically acceptable salt form thereof, and a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of Compound A or a pharmaceutically acceptable salt form thereof for treating cancer or an immunological disease.

Other embodiments of the invention are directed to using of a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of BTK inhibitor or a pharmaceutically acceptable salt form thereof, and a therapeutically effective dose ranging from about 25 to 1000 mg, alternatively from about 25 to 500 mg, alternatively from about 25 to 400 mg, alternatively from about 25 to 300 mg, alternatively from about 50 to 1000 mg of Compound A or a pharmaceutically acceptable salt form thereof in the manufacture of a medicament for treating a disorder or condition that is affected by the inhibition of MALT1. In specific embodiments, the BTK inhibitor used in these methods is ibrutinib or Roche BTKi RN486. Alternatively, the BTK inhibitor is acalabrutinib or zanubrutinib. In yet another embodiment, the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide (Compound B).

One embodiment of the invention is a method of treating diffuse large B-cell lymphoma (DLBCL) comprising administering a therapeutically effective dose of Compound A in combination with a therapeutically effective dose of BTK inhibitor. In some embodiments, the DLBCL is relapsed or refractory to a prior treatment. In certain embodiments, the method comprises providing a composition comprising Compound A and BTK inhibitor. In one embodiment, the method of treating diffuse large B-cell lymphoma (DLBCL) comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the DLBCL is the activated B cell like (ABC) subtype of diffuse large B-cell lymphoma (DLBCL). In some embodiments, the DLBCL is germinal center B cell like (GCB) subtype of diffuse large B-cell lymphoma (DLBCL). In some embodiments, the DLBCL is non-germinal center B cell like (non-GCB) subtype of diffuse large B-cell lymphoma (DLBCL). In some embodiments, the method achieves an ORR of at least about 30% in a group of subjects diagnosed with DLBCL.

One embodiment of the invention is a method of treating Waldenström Macroglobulinemia (WM) comprising administering a therapeutically effective dose of Compound A in combination with a therapeutically effective dose of BTK inhibitor. In some embodiments, the WM is relapsed or refractory to a prior treatment. In certain embodiments, the method comprises providing a composition comprising Compound A and BTK inhibitor. In one embodiment, the method of treating Waldenström Macroglobulinemia (WM) comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the method achieves an ORR of at least about 30% in a group of subjects diagnosed with WM.

One embodiment of the invention is a method of treating non-Hodgkin's lymphoma (NHL) comprising administering a therapeutically effective dose of Compound A in combination with a therapeutically effective dose of BTK inhibitor. In some embodiments, the NHL is relapsed or refractory to a prior treatment. In certain embodiments, the method comprises providing a composition comprising Compound A and BTK inhibitor. In one embodiment, the method of treating NHL comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the method achieves an ORR of at least about 30% in a group of subjects diagnosed with NHL.

One embodiment of the invention is a method of treating mantle cell lymphoma (MCL) comprising administering a therapeutically effective dose of Compound A in combination with a therapeutically effective dose of BTK inhibitor. In some embodiments, the MCL is relapsed or refractory to a prior treatment. In certain embodiments, the method comprises providing a composition comprising Compound A and BTK inhibitor. In one embodiment, the method of treating MCL comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the method achieves an ORR of at least about 30% in a group of subjects diagnosed with MCL.

One embodiment of the invention is a method of treating marginal zone lymphoma (MZL) comprising administering a therapeutically effective dose of Compound A in combination with a therapeutically effective dose of BTK inhibitor. In some embodiments, the MZL is relapsed or refractory to a prior treatment. In certain embodiments, the method comprises providing a composition comprising Compound A and BTK inhibitor. In one embodiment, the method of treating MZL comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the method achieves an ORR of at least about 30% in a group of subjects diagnosed with MZL

One embodiment of the invention is a method of treating follicular lymphoma comprising administering a therapeutically effective dose of Compound A in combination with a therapeutically effective dose of BTK inhibitor. In some embodiments, the follicular lymphoma is relapsed or refractory to a prior treatment. In certain embodiments, the method comprises providing a composition comprising Compound A and BTK inhibitor. In one embodiment, the method of treating follicular lymphoma comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the method achieves an ORR of at least about 30% in a group of subjects diagnosed with follicular lymphoma.

One embodiment of the invention is a method of treating transformed follicular lymphoma comprising administering a therapeutically effective dose of Compound A in combination with a therapeutically effective dose of BTK inhibitor. In some embodiments, the transformed follicular lymphoma is relapsed or refractory to a prior treatment. In certain embodiments, the method comprises providing a composition comprising Compound A and BTK inhibitor. In one embodiment, the method of treating transformed follicular lymphoma comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the method achieves an ORR of at least about 30% in a group of subjects diagnosed with transformed follicular lymphoma.

One embodiment of the invention is a method of treating chronic lymphocytic leukemia (CLL) comprising administering a therapeutically effective dose of Compound A in combination with a therapeutically effective dose of BTK inhibitor. In some embodiments, the CLL is relapsed or refractory to a prior treatment. In certain embodiments, the method comprises providing a composition comprising Compound A and BTK inhibitor. In one embodiment, the method of treating chronic lymphocytic leukemia (CLL) comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the method achieves an ORR of at least about 30% in a group of subjects diagnosed with CLL.

One embodiment of the invention is a method of small lymphocytic lymphoma (SLL) comprising administering a therapeutically effective dose of Compound A in combination with a therapeutically effective dose of BTK inhibitor. In some embodiments, the SLL is relapsed or refractory to a prior treatment. In certain embodiments, the method comprises providing a composition comprising Compound A and BTK inhibitor. In one embodiment, the method of treating SLL comprises: a step of administering a therapeutically effective dose of Compound A; and a step of administering a therapeutically effective dose of BTK inhibitor. The BTK inhibitor may be administered before Compound A, after Compound A or concurrently with Compound A. In certain embodiments, the therapeutically effective dose of Compound A and BTK inhibitor ranges from about 25 to 1000 mg, alternatively from about 25 to 500 mg, alternatively from about 25 to 400 mg, alternatively from about 25 to 300 mg, alternatively from about 50 to 1000 mg. In some embodiments, the therapeutic effective dose of Compound A is about 100-300 mg QD, and the therapeutic effective dose of BTK inhibitor is about 140-560 mg QD. In some embodiments, the therapeutic effective dose of Compound A is about 100-300 mg QD, and the therapeutic effective dose of BTK inhibitor is about 140-560 mg BD. In some embodiments, the therapeutic effective dose of Compound A is about 100-300 mg BD for 7 days followed by 100-300 mg QD, and the therapeutic effective dose of BTK inhibitor is about 140-560 mg QD. In some embodiments, the therapeutic effective dose of Compound A is about 100-300 mg BD for 7 days followed by 100-300 mg QD, and the therapeutic effective dose of BTK inhibitor is about 140-560 mg BD. In some embodiments, the therapeutic effective dose of Compound A is about 200 mg QD, and the therapeutic effective dose of BTK inhibitor is about 560 mg QD. In some embodiments, the therapeutic effective dose of Compound A is about 300 mg QD, and the therapeutic effective dose of BTK inhibitor is about 560 mg QD. In some embodiments, the therapeutic effective dose of Compound A is about 200 mg QD, and the therapeutic effective dose of BTK inhibitor is about 420 mg QD. In some embodiments, the therapeutic effective dose of Compound A is about 300 mg QD, and the therapeutic effective dose of BTK inhibitor is about 420 mg QD. In some embodiments, the therapeutic effective dose of Compound A is about 150 mg BID, and the therapeutic effective dose of BTK inhibitor is about 280 mg BID. In some embodiments, the therapeutic effective dose of Compound A is about 100 mg BID, and the therapeutic effective dose of BTK inhibitor is about 210 mg BID. In some embodiments, the therapeutic effective dose of Compound A is about 150 mg BID, and the therapeutic effective dose of BTK inhibitor is about 210 mg BID. In other embodiments, any of the therapeutically effective doses, administration intervals and/or dosage cycles described herein may be used. In any of these embodiments, the BTK inhibitor used in these methods is ibrutinib, Roche BTKi RN486, acalabrutinib, zanubrutinib, or N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide. In some embodiments, the method achieves an ORR of at least about 30% in a group of subjects diagnosed with SLL.

In some embodiments, the subject may have received at least 2 prior lines of therapy prior to administration of Compound A and BTK inhibitor. In some embodiments, the subject may have received first line chemotherapy and at least 1 subsequent line of systemic therapy, including autologous stem cell transplantation (ASCT), prior to administration of Compound A and BTK inhibitor. In some embodiments, the subject may have received at least 2 prior lines of systemic therapy, including a standard anti CD20 antibody, prior to administration of Compound A and BTK inhibitor. In some embodiments, the subject may have received ASCT, prior to administration of Compound A and BTK inhibitor. In some embodiments, the subjects may not be eligible for ASCT. In some embodiments, the combination of Compound A and the BTK inhibitor is used as a front line therapy.

In another embodiment of the present invention, Compound A and the BTK inhibitor may be employed in combination with one or more other medicinal agents, more particularly with other anti-cancer agents, e.g. chemotherapeutic, anti-proliferative or immunomodulating agents, or with adjuvants in cancer therapy, e.g. immunosuppressive or anti-inflammatory agents.

In some embodiments, the BTK inhibitors disclosed herein and Compound A may exhibit different PK profiles when administered together due to potential drug-drug interactions. In some embodiments, when the BTK inhibitor is administered in combination with Compound A, the Cmax of BTK inhibitor may increase by about 20%, about 25%, about 30%, about 35%, about 40%, about 45%, about 50%, about 55%, about 60% when compared to Cmax of BTK inhibitor administered alone. In some embodiments, when the BTK inhibitor is administered in combination with Compound A, the AUC of BTK inhibitor may increase by about 20%, about 25%, about 30%, about 35%, about 40%, about 45%, about 50%, about 55%, about 60%, about 65%, about 70%, about 75%, about 80%, when compared to AUC of BTK inhibitor administered alone. In some embodiments, the BTK inhibitor is Ibrutinib. In some embodiments, the BTK inhibitor is Compound B.

In some embodiments, a method of treating cancer in a subject comprises administering about 300 mg of Compound A in combination with a BTK inhibitor to a subject, wherein the amount of BTK inhibitor that is administered will not exceed about 150 mg, about 175 mg, about 200 mg, about 210 mg, about 225 mg, about 250 mg, about 280 mg, or about 300 mg. In some embodiments, the BTK inhibitor is Compound B or Ibrutinib. In some embodiments, the cancer is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

In some embodiments, a method of treating cancer in a subject comprises administering about 200 mg of Compound A in combination with a BTK inhibitor to a subject, wherein the amount of BTK inhibitor that is administered will not exceed about 125 mg, about 150 mg, about 175 mg, about 200 mg, about 210 mg, about 225 mg, about 250 mg, about 280 mg, or about 300 mg. In some embodiments, the BTK inhibitor is Compound B or Ibrutinib. In some embodiments, the cancer is selected from non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

It will be appreciated that variations to the foregoing embodiments of the invention can be made while still falling within the scope of the invention. Each feature disclosed in this specification, unless stated otherwise, may be replaced by alternative features serving the same, equivalent, or similar purpose. Thus, unless stated otherwise, each feature disclosed is one example only of a generic series of equivalent or similar features.

All possible combinations of the above-indicated embodiments are considered to be embraced within the scope of this invention.

According to an embodiment, the invention provides combinations as described herein.

According to an embodiment, the invention provides combinations as described herein for use as a medicament.

According to an embodiment, the invention provides combinations as described herein for the manufacture of a medicament.

According to an embodiment, the invention provides combinations as described herein for the manufacture of a medicament for the treatment of any one of the disorders or conditions mentioned herein.

According to an embodiment, the invention provides combinations as described herein for use in the treatment of any one of the disorders or conditions as described herein.

According to an embodiment, the invention provides combinations as described herein for use in treating of any one of the disorders or conditions as described herein.

All embodiments described herein for methods for treating, are also applicable for use in treating.

All embodiments described herein for methods for treating a disorder or condition, are also applicable for use in treating said disorder or condition.

All embodiments described herein for use in treating a disorder or condition, are also applicable for methods for treating said disorder or condition.

All embodiments described herein for methods for treating a disorder or condition, are also applicable for use in a method for treating said disorder or condition.

All embodiments described herein for use in a method for treating a disorder or condition, are also applicable for methods for treating said disorder or condition.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### EXAMPLES

The following examples of the invention are to further illustrate the nature of the invention. It is believed that one of ordinary skill in the art can, using the preceding description and the following illustrative examples, make and utilize the present invention and practice the claimed methods. It should be understood that the following examples do not limit the invention and that the scope of the invention is to be determined by the appended claims.

Consistent with the use of the term “Compound A” above, “Compound A” as used throughout these examples is 1-(1-oxo-1,2-dihydroisoquinolin-5-yl)-5-(trifluoromethyl)-N-[2-(trifluoromethyl)pyridin-4-yl]-1H-pyrazole-4-carboxamide.

As used in the Examples 2 and 3, “Compound B” is the BTK inhibitor N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide.

### Example 1: In Vitro Combinations of a MALT1 Inhibitor with BTK Inhibitors in ABC-DLBCL Cell Lines

The viability of ABC-DLBCL cell lines after treatment with Compound A in combination with ibrutinib was evaluated in vitro. ABC-DLBCL cell lines (OCI-Ly10, TMD8, and HBL1) were grown in 96-well plates and treated with a matrix of seven scalar concentrations of Compound A (20-0.027 μM) and six scalar concentrations of ibrutinib (1-[(3R)-3-[4-amino-3-(4-phenoxyphenyl)pyrazolo[3,4-d]pyrimidin-1-yl]piperidin-1-yl]prop-2-en-1-one) (6.4-0.026 nM).

Potential combination effects were evaluated after 4 days treatment using Cell Titer Glo. Data from ≥3 repeats were combined and analyzed for synergy assessment using the HSA or Generalized Loewe model using the extended BIGL package (modelling variance).

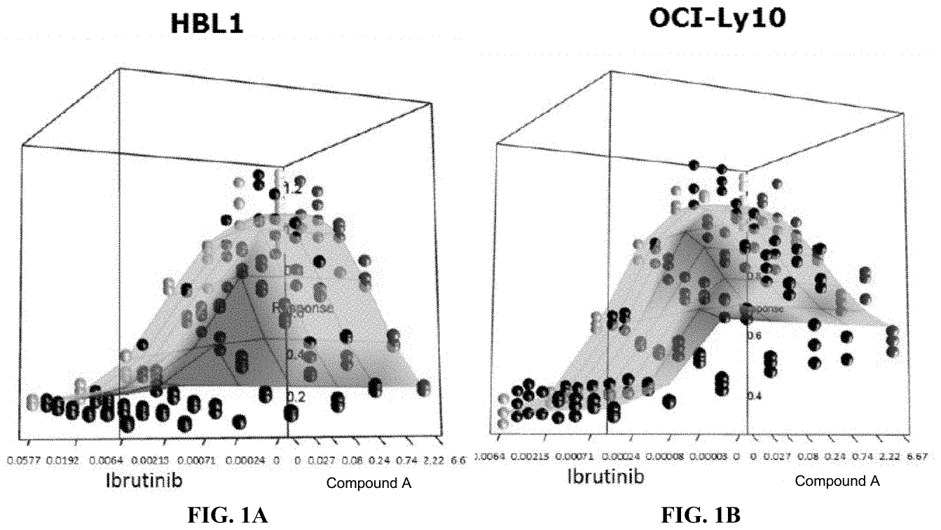

A synergistic effect was observed at specific concentrations of ibrutinib and Compound A in OCI-Ly10 using both models. Data shown for HSA model in FIG. 1B. With reference to FIGS. 1A and 1B, the concentrations of Ibrutinib and Compound A are shown on the X-axis (μM). Minor synergistic effects were seen in HBL1 cells (data shown for HSA model in FIG. 1A) while synergistic effects in TMD8 cells were only identified when using the less stringent HSA model (data not shown). Similar synergistic effects (data not shown) were observed using the Roche BTKi RN486 in combination with Compound A.

### Example 2: In Vitro Activity of the Combination of Compound a and the BTK Inhibitor N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide

The studies in this Example provide a characterization of combination of the BTK inhibitor Compound B and the MALT1 inhibitor Compound A in vitro. The objective of the studies was the evaluation of antiproliferative activity after treatment with a combination of the BTK inhibitor Compound B and the MALT1 inhibitor Compound A in vitro. A panel of DLBCL and MCL cell lines was evaluated for cell proliferation after treatment with either monotherapy of Compound B or Compound A or a combination of both agents in dose-response. Additive or synergistic effects were also evaluated.

Compound B (N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide) is an orally active, small molecule that is a potent, selective, and irreversible covalent BTK inhibitor. Compound B potently inhibits BTK kinase activity in cellular assays. Compound B inhibits growth of CD79b-mutant DLBCL (Diffuse Large B Cell Lymphoma) cell lines in vitro.

Compound A is an allosteric inhibitor of MALT1 protease with a mixed-type mechanism. Compound A potently inhibits MALT1 protease activity in biochemical and cellular assays. Compound A inhibits growth of CD79b-mutant DLBCL and ibrutinib-resistant DLBCL harbouring BTK C481S or CARD11 mutations in vitro. As will be shown below, the combination of Compound B and Compound A results in synergistic activity in CD79b-mutant DLBCL and a subset of MCL (Mantle Cell Lymphoma) cellular models.

### Materials and Methods

Compound A and Compound B were used throughout the in vitro and cellular assessment of activity. All small molecules were prepared as 100% dimethyl sulfoxide (DMSO) stocks as indicated. A final concentration of 0.25% DMSO was used as a control. High Control=Cells+DMSO 0.25%.

The testing used the following reagents: L-Glutamine (Cat #G7513) (Gibco); RPMI 1640 (Cat #R0883) (Gibco); DMSO (D2650) (Gibco), RPMI Glutamax (Cat #2183129) (Gibco), Gentamicin (Cat #15750-037) (Gibco) FBS (Cat #S1810-500) (Biowest); clear flat bottom black 96 well (Cat #3904) (Corning); CellTiter-Glo® Luminescent Cell Viability Assay (G7573) (Buffer Cat #G756B and Substrate Cat #G755B) (Promega).

The following cell lines were used: OCI-LY-3 and HBL-1 (Dr. Miguel A Piris, Hospital Universitario Marques de Valdecilla, Santander, Spain); OCI-LY-10 (UHN (University Hospital Network); TMD-8 (Tokyo University); REC-1 (DSMZ ACC 584); JEKO-1 (DSMZ ACC 553); MINO (DSMZ ACC 687); and MAVER-1 (ATCC CRL-3008). The culture media used with the cell lines are described below.

### Protocol for Assessing Inhibition of DLBCL Cancer Proliferation

The viability of ABC-DLBCL cell lines after treatment with Compound A in combination with Compound B was evaluated in vitro. A panel of 4 B-cell lymphoma lines was treated with different doses of both compounds. The following ABC-DLBCL cell lines were tested: OCI-Ly10; TMD8; OCI-Ly3; and HBL1. The cell lines were grown in 96-well plates and treated with a matrix of seven scalar concentrations of Compound A (20-0.027 M) and six scalar concentrations of Compound B (0.5-0.002 M) and all combinations thereof. This checkerboard design was repeated on up to four separate plates. The final concentration of DMSO was 0.25%. After an incubation of four days at 37° C. and 5% CO₂, cell viability was determined by adding CellTiter-Glo®. Luminescence was read on an Envision device and readouts were used to calculate potential combination effects. Data from 2-4 independent experiments were combined and analysed for synergy assessment using the HSA or Generalized Loewe model using the extended BIGL package (modelling variance).

### Protocol for Assessing Inhibition of MCL Cancer Cell Proliferation

The viability of Mantle cell lymphoma (MCL) cell lines after treatment with Compound A in combination with Compound B was evaluated in vitro with the same method used for DLBCL cells. A panel of 4 MCL cell lines were treated with different doses for both compounds (using the same dosing as above). The following MCL cell lines were tested: REC-1; JEKO-1; MINO; and MAVER-1. REC-1 was evaluated which is known to be dependent on the NF-κB pathway and was shown to be sensitive to BTK and MALT1 inhibitor monotherapy in vitro.

### Data Analysis

For the assessment of combination effects on DLBCL or MCL cancer cell proliferation, the observed combination effects were evaluated using the public available BIGL (Biochemically Intuitive Generalized Loewe Model) R package which measures the evidence in the data, in the presence of variability, against certain null models derived from the monotherapies. In first step, monotherapies were fitted with 4PL models with extra constraint of common baseline between two agents. When no activity was observed, a straight line was fitted through the monotherapy data. In a second step, hypothesis tests at 5% significance level were performed which basically contrast the observed readouts of the combination experiments with those predicted from the null model derived from the monotherapies where both HSA (Highest Single Agent) 17 and generalized Loewe18 were assessed for these studies.

### Results

### Inhibition of DLBCL Cancer Cell Proliferation

The combination effects of the BTK inhibitor Compound B and Compound A were analyzed from multiple in vitro experiments. Specifically, the combination effects were assessed in the following cell lines: OCI-Ly10; HBL1; TMBD8; and OCI-Ly3.

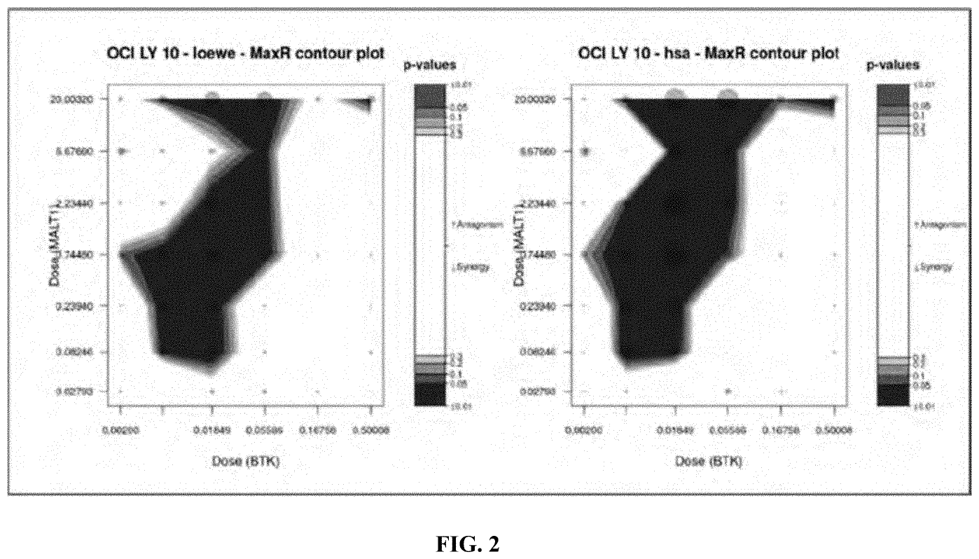

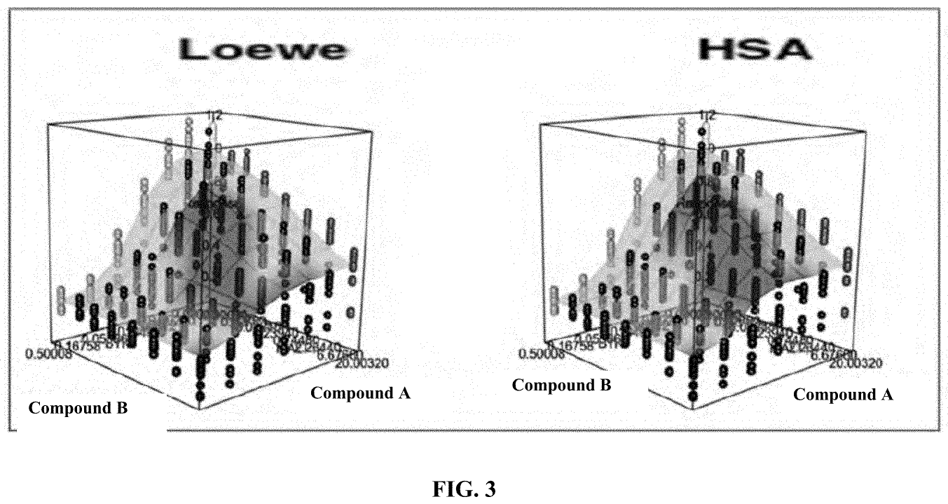

To analyze combination effects in OCI-Ly10 cells, four independent experiments with similar monotherapy activity were combined. Monotherapy of Compound B was observed to result in up to 75% proliferation inhibition while Compound A led to approximately 50% proliferation inhibition in OCI-Ly10 cells at the highest concentration tested. Strong synergy was observed in conditions of medium or high doses of both inhibitors in the CD79b-mutant OCI-Ly10 cellular model. Statistically significant synergy was observed using both analysis methods, Generalized Loewe and HSA (FIG. 2 & FIG. 3).

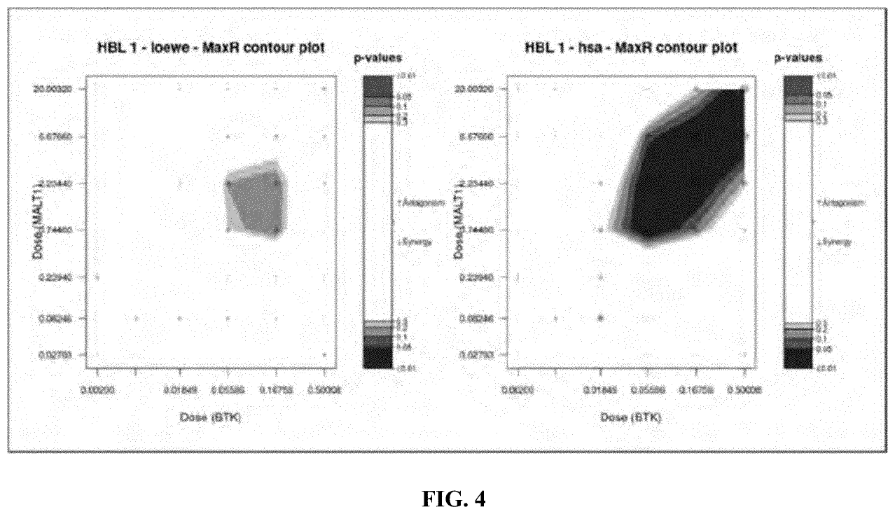

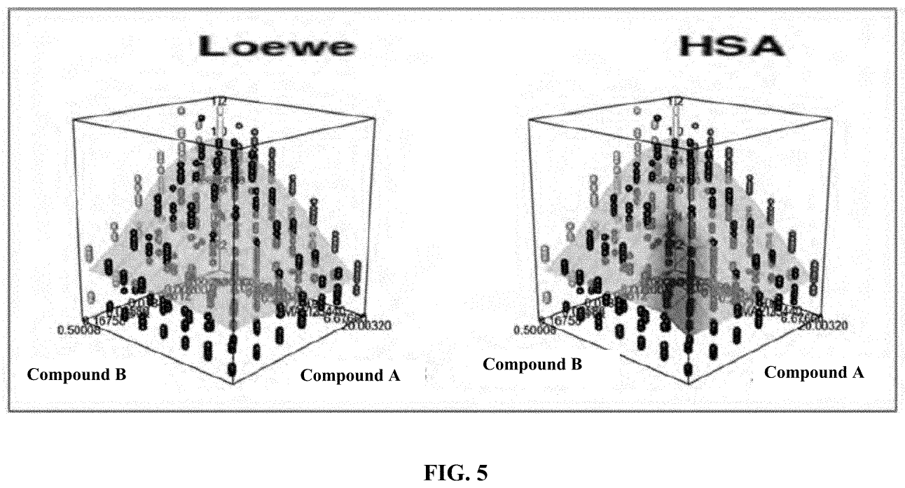

To analyze combination effects in HBL1 cells, three independent experiments with similar monotherapy activity were combined. Monotherapy of Compound B as well as monotherapy of Compound A was observed to result in up to 75% proliferation inhibition in HBL1 cells at the highest concentration tested. A fourth experiment was excluded from the analysis as the monotherapy dose response for the BTK inhibitor Compound B was a clear outlier showing no anti-proliferative effect likely due to a technical error. Synergy was observed in conditions of medium or high doses of both inhibitors in the CD79b-mutant HBL1 cellular model. Statistically significant synergy was observed using both analysis methods, Generalized Loewe and HSA, but less prominent or limited to a few dose concentrations for the more stringent Loewe model (FIGS. 4 & 5).

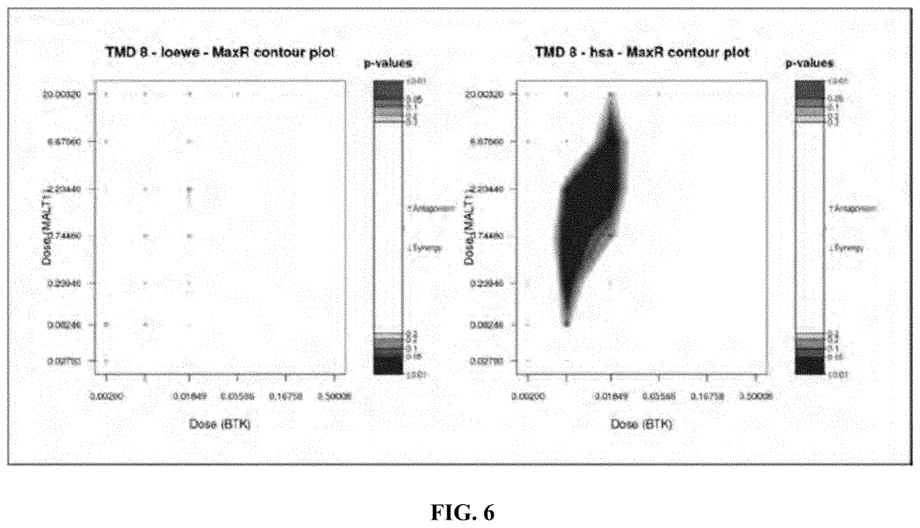

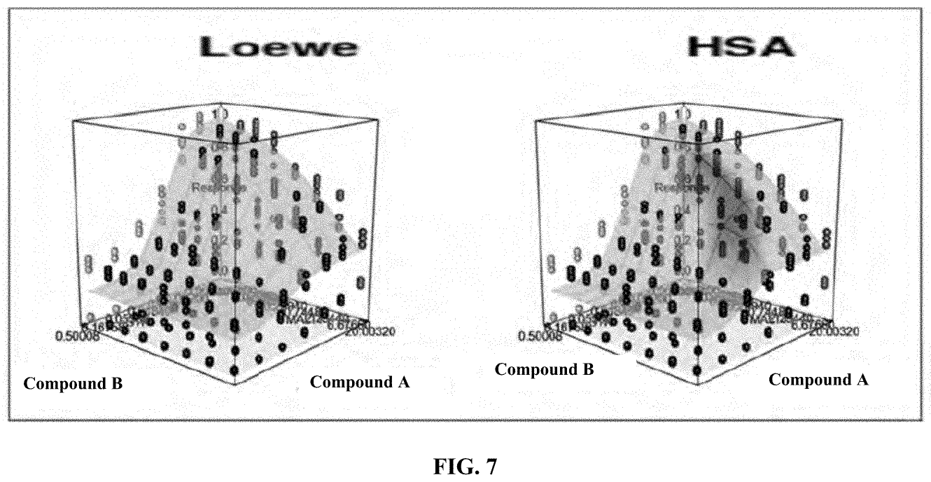

To analyze combination effects in TMD8 cells, two independent experiments with similar monotherapy activity were combined. Monotherapy of Compound B was shown to result in up to 80/proliferation inhibition while Compound A led to approximately 55% proliferation inhibition in TMD8 cells at the highest concentration tested. Two other experiments were excluded from the analysis as the monotherapy dose responses for the BTK inhibitor Compound B were clear outliers showing either no anti-proliferative effect or extremely strong activity likely due to technical errors. Synergy was observed in conditions of medium or high doses of Compound A and lower or medium doses of Compound B in the CD79b-mutant TMD8 cellular model. Statistically significant synergy was observed using the HSA analysis method (FIG. 6 & FIG. 7). Due to the higher variability between the different independent experiments, synergy assessments were more difficult. If individual runs are analyzed separately, statistically significant synergy is also observed with the Generalized Loewe model.

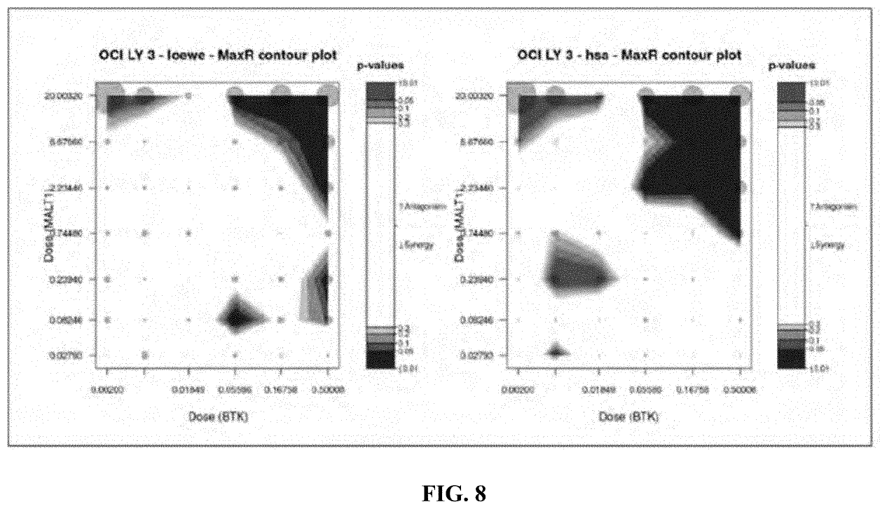

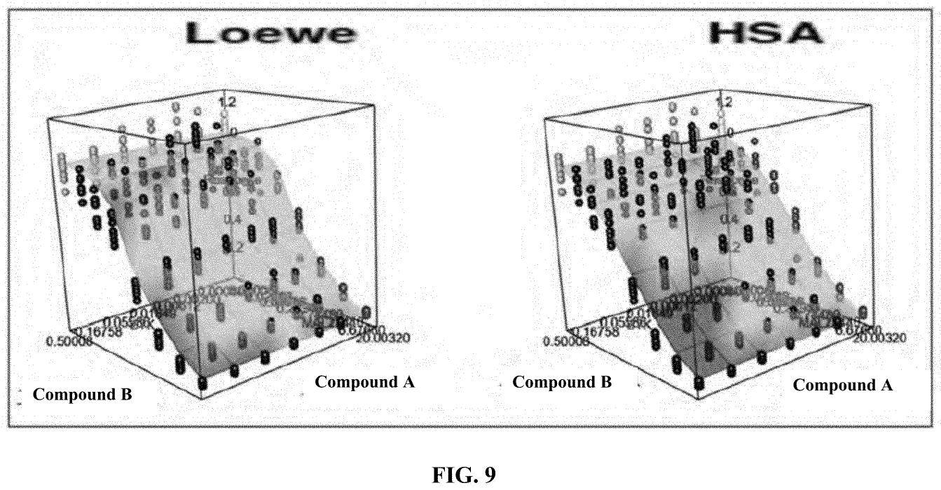

To analyze combination effects in OCI-Ly3 cells, two independent experiments with similar monotherapy activity were combined. Monotherapy of Compound B did not inhibit proliferation while Compound A led to up to 95% proliferation inhibition in OCI-Ly3 cells at the highest concentration tested. While some synergy was observed it was limited to high doses of Compound A and high doses of Compound B in the CARD11-mutant OCI-Ly3 cellular model (FIG. 8). Interestingly, there were also some areas of antagonistic activity observed, in particular, using the HSA analysis method. The observed effects are statistically significant (HSA & Loewe) but as seen in the 3D plots the synergistic or antagonistic effects are minor (FIG. 9) and much smaller compared to other clear synergy calls in the CD79b-mutant cell lines.

### Inhibition of MCL Cancer Cell Proliferation

Combination effects of the BTK inhibitor Compound B and Compound A were analyzed from multiple in vitro experiments. Specifically, the combination effects were assessed in the following MCL cancer cell lines: REC-1; JEKO-1; MINO; and MAVER-1.

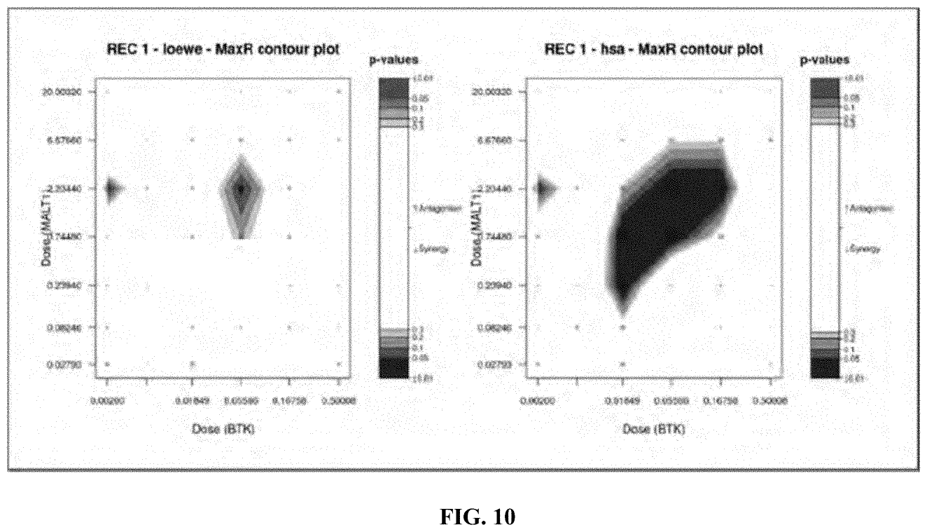

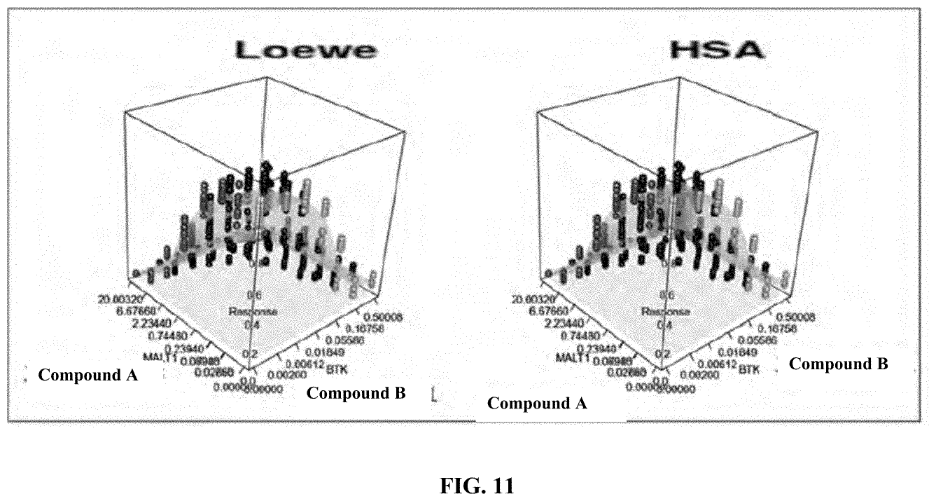

To analyze combination effects in REC-1 cells, three independent experiments with similar monotherapy activity were combined. Both monotherapy of Compound B and Compound A resulted in up to 95% proliferation in REC-1 cells at the highest concentration tested. A fourth experiment was excluded from the analysis as the monotherapy dose response for the BTK inhibitor Compound B and MALT1 inhibitor Compound A were clear outliers with strong shift towards decreased max effect for Compound B and overall shift in decreased potency for Compound A. Synergy was observed in conditions of medium or high doses of both inhibitors in the REC-1 cellular model. Statistically significant synergy was observed using the HSA analysis method, as well as for a few concentrations with the Generalized Loewe analysis method (FIG. 10 & FIG. 11).

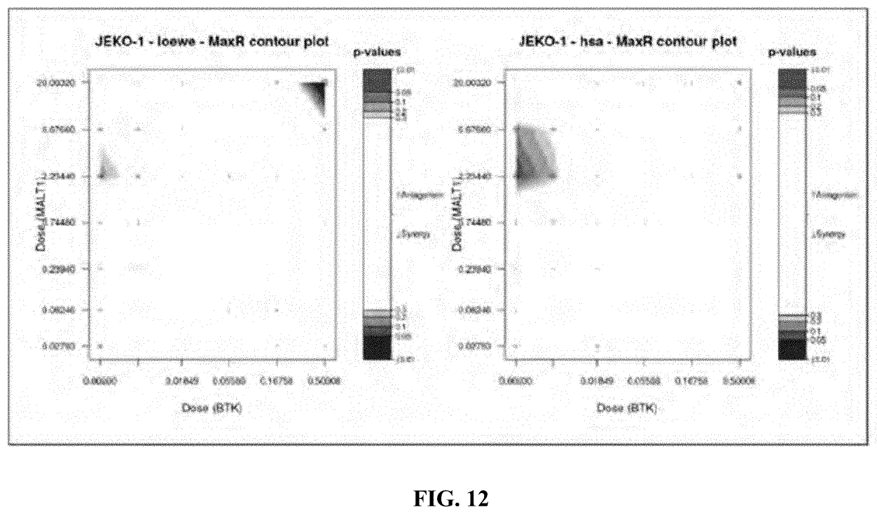

To analyze combination effects in JEKO-1 cells, three independent experiments with similar monotherapy activity were combined. Monotherapy of Compound B only showed minor inhibition of proliferation (˜25%) while Compound A led to up to 60% proliferation inhibition in JEKO-1 cells with significant inhibition only seen at the highest concentrations tested. No synergistic or antagonistic effects were observed in any conditions in the JEKO-1 cellular model (FIG. 12).

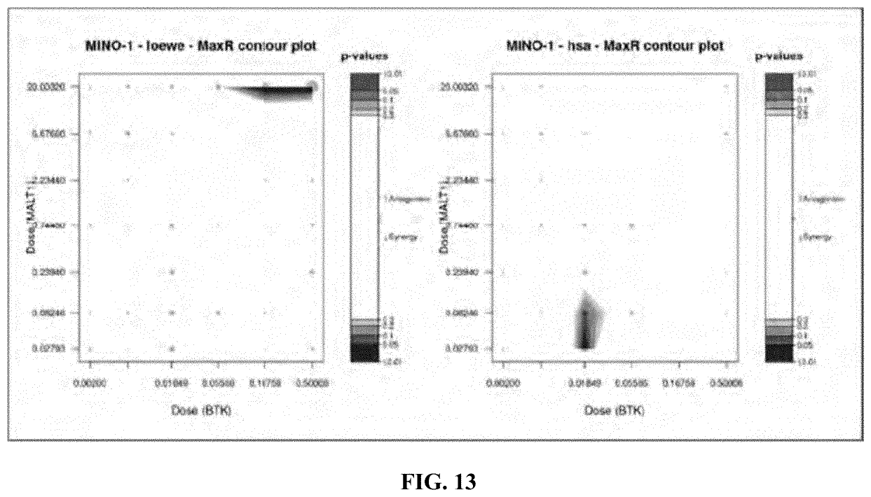

To analyze combination effects in MINO cells, three independent experiments with similar monotherapy activity were combined. Monotherapy of Compound B only showed minor inhibition of proliferation (˜30%) while Compound A led to up to 45% proliferation inhibition in MINO cells with significant inhibition only seen at the highest concentrations tested. No clear synergistic or antagonistic effects were observed in any conditions in the MINO cellular model (FIG. 13).

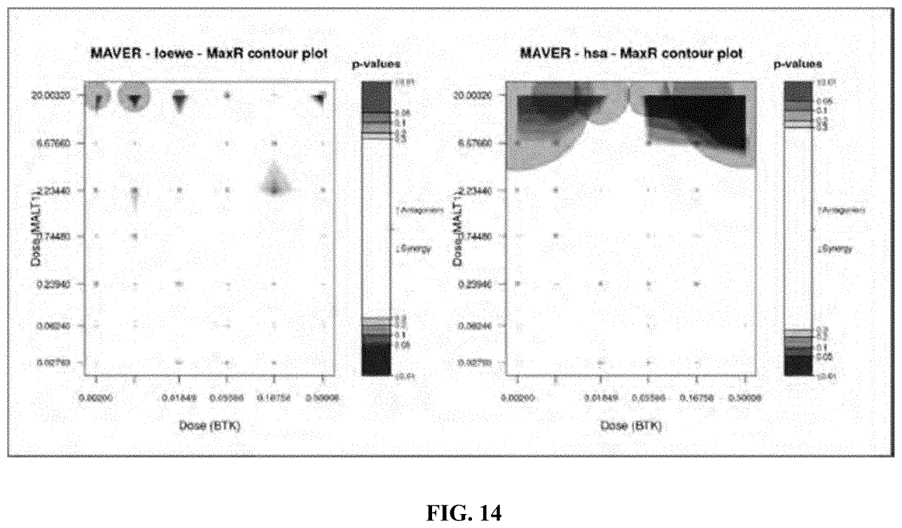

To analyze combination effects in MAVER-1 cells, three independent experiments with similar monotherapy activity were combined. Monotherapy of Compound B only showed no to minor inhibition of proliferation; Compound A led to up to 50% proliferation inhibition in MAVER-1 cells with significant inhibition only seen at the highest concentrations tested. No clear synergistic or antagonistic effects were observed in any conditions in the MAVER-1 cellular model despite at the highest doses of both inhibitors after analysis with the HSA model which may be caused by off-target activity (FIG. 14).

### Discussion

The classical nuclear factor kappa-light-chain-enhancer of activated B cells (NF-κB) signaling pathway is constitutively activated in many B cell lymphomas and a hallmark of ABC-DLBCL (Activated B-Cell Diffuse Large B-Cell Lymphoma). Bruton's Tyrosine Kinase (BTK) and Mucosa-associated lymphoid tissue lymphoma translocation protein 1 (MALT1) are both key mediators of the classical nuclear factor kappa-light-chain enhancer of activated B cells (NF-κB) signaling pathway and play a critical roles in the activated B cell subtype-diffuse large B-cell lymphoma (ABC-DLBCL). BTK inhibitors have been extensively investigated for the treatment of B-cell hematological malignancies and two small molecule inhibitors, ibrutinib and acalabrutinib, are currently marketed as anticancer agents for multiple B cell malignancies.

Compound B is an orally active, small molecule that is a potent, selective, and irreversible covalent BTK inhibitor. Compound A is an allosteric inhibitor of MALT1 protease. This example provides in vitro studies evaluating the combination of Compound B and Compound A in dose response in multiple ABC-DLBCL and MCL cell lines. Synergistic effects were observed in CD79b-mutant ABC-DLBCL cell lines (OCI-Ly10, HBL1, and TMD8) which are sensitive to both agents in monotherapy. Minor synergistic effects were observed in the CARD11-mutant ABC-DLBCL cell line OCI-Ly3. Synergistic effects were further observed in the MCL cell line REC1 which is sensitive to both agents in monotherapy. No synergistic or antagonistic effects were observed in MCL cell lines (JEKO-1, MINO, and MAVER-1) that show only limited response to both agents as monotherapy. The generated in vitro data support a first in-human trial of a combination of Compound B and Compound A in patients with ABC-DLBCL lymphomas driven by CD79b mutations as well as a subset of MCL patients.

Accordingly, using a combination of the BTK inhibitor Compound B and the MALT1 inhibitor Compound A is a valid strategy for the treatment of patients with ABC-DLBCL lymphomas, in particular, in such lymphomas that are driven by CD79b mutations, and MCL patients.

### Example 3: Efficacy of BTK Inhibitor Monotherapy and Combination with Compound a in Diffuse Large B-Cell Lymphoma Xenografts in NSG Mice

BTK is part of the B-cell antigen receptor signaling pathway and plays an essential role in B-cell maturation, differentiation, and function of mature B-cells. Abdalla et al., Immunol Rev, 2009; 228(1):58-73. BTK plays a crucial role in oncogenic signaling and is key to proliferation and survival of tumorigenic cells in many B cell malignancies. Rudi et al., Nat Rev Cancer, 2014; 14(4): 219-32. The BTK inhibitor ibrutinib has shown beneficial anti-tumor effects in many B cell malignancies, however resistance may occur (Shah et al. Trends Cancer. 2018; 4:197-206) necessitating the development of further combination therapies to improve anti-tumor activity.

Compound B is an oral covalent Bruton's tyrosine kinase (BTK) inhibitor. BTK inhibition results in downstream B cell antigen receptor (BCR) signaling blockade, leading to disrupted proliferation and tumor cell killing in many B cell malignancies. Compound B inhibits proliferation of ABC-DLBCL cell lines bearing cluster of differentiation (CD)79b mutations, with an IC₅₀ of 18 nM for OCI-LY10. Compound B is orally bioavailable with moderate clearance and a short (0.7 hour) half-life in NSG mice.

MALT1 is a key mediator of the classical NF-κB signaling pathway and has been shown to play a critical role in ABC-DLBCL.12 Libermann et al. Mol Cell Biol. 1990 May; 10(5): 2327-34. MALT1 is a unique paracaspase that transduces signals from the B-cell receptor and T-cell receptor. MALT1 possesses two functions: a scaffolding function to recruit NF-κB signaling proteins; and a protease function to cleave and inactivate inhibitors of the NF-κB signaling pathway. It is hypothesized that MALT1 inhibition will target ABC-DLBCL tumors with CD79 or caspase recruitment domain-containing protein 11 (CARD11) mutations as well as DLBCL, chronic lymphocytic leukemia (CLL), and mantle cell lymphoma, Waldenström macroglobulinemia, and tumors with acquired resistance to BTK inhibitors such as IMBRUVICA® (ibrutinib). Cao et al., J Biol Chem. 2006 Sep. 8; 281(36):26041-50; Fontan et al., Cancer Cell. 2012; 22(6):812-24; Hailfinger et al., Proc Natl Acad Sci USA. 2009; 106(47):19946-51; Nagel et al., Cancer Cell. 2012; 22(6):825-37; and Shat et al. (supra).

Compound A is an allosteric MALT1 protease inhibitor developed to target B cell lymphomas dependent on the classical NF-κB signaling pathway. Compound A inhibits proliferation of ABC-DLBCL cell lines bearing CD79b or CARD11 mutations, with an IC50 of 0.332 μM in OCI-LY10 cells. Compound A is orally bioavailable (% F>90 in mice) with slow to moderate clearance and half-life in mice exceeding 5 hours (T_{1/2}=5.74 hr in NSG mice).

The combination of the BTK inhibitor Compound B and the MALT1 inhibitor Compound A was evaluated in dose response in multiple ABC-DLBCL cell lines (see Example 2 above). Synergistic effects were observed in CD79b-mutant ABC-DLBCL cell lines (OCI-Ly10, HBL1, and TMD8) which are sensitive to both agents in monotherapy.

The objective of the studies shown in this example was the evaluation of in vivo pharmacodynamic effects and anti-tumor efficacy of Compound B alone and in combination with Compound A in the OCI-LY10 ABC-DLBCL xenograft model. This model is characterized by the constitutive activation of the NF-κB signaling pathway, driven by CD79b mutation. To reach optimal serum exposures in mouse tumor models, Compound A was administered BID in the current studies, while both QD and BID dosing schedules were tested for Compound B. In particular, the pharmacodynamic effect (PD) and anti-tumor efficacy of Compound B was evaluated in a human activated B cell subtype-diffuse large B-cell lymphoma (ABC-DLBCL) xenograft model in NOD.Cg-Prkdc^{scid}IL2^{rgtmlWjl}/SzJ gamma (NSG) mice, either as a monotherapy or in combination with orally administration of an allosteric protease inhibitor of Mucosa-associated lymphoid tissue lymphoma translocation protein 1 (MALT1) inhibitor (Compound A).

### Materials and Methods

Compound B (a small BTK inhibitor, dihydrate salt) and was formulated as a solution for oral (PO) administration in PEG400 or PEG400 with 10% 6:4 linear random copolymer of 1-vinyl-2-pyrrolidone and vinyl acetate (PVP-VA64). The compound was formulated every week, by adding the required volume of PEG400 or PEG400/PVP-VA64 to pre-weighed compound and stirring until dissolved. To limit the amount of PEG-400, dose volumes of 5 mL/kg were administered in Study 1, Study 2, and Study 3, while a dose volume of 2.5 mL/kg was used for Compound B in Study 4. Compound A (a small molecule MALT1 inhibitor, monohydrate salt) was formulated as a solution for oral (PO) administration in PEG400. Compound was formulated every week, by adding the required volume of PEG400 to pre-weighed compound and stirring until dissolved. Dose volumes administered for MALT1 inhibitor were 3.33 mL/kg across all combination studies. Both formulated compounds were stored at room temperature protected from light.

### Animals

For all studies, female NSG mice (Jackson Laboratory) were used when they were approximately 6 to 8 weeks of age and weighed approximately 20 grams. All animals were allowed to acclimate and recover from any shipping-related stress for a minimum of 5 days prior to experimental use. Autoclaved water and irradiated food (NIH 31 Modified and Irradiated Lab Diet®) were provided ad libitum, and the animals were maintained on a 12-hours light and dark cycle. Cages, bedding, and water bottles were autoclaved before use and changed weekly. All experiments were carried out in accordance with The Guide for the Care and Use of Laboratory Animals and were approved by the Institutional Animal Care and Use Committee. All studies were conducted in compliance with the external animal research policies of Johnson and Johnson.

### Critical Reagents

Tables 2 and 3 show the critical reagents used for the studies in this Example.

TABLE 2
Reagents for Tissue Culture and Cell Injection

| Reagent | Catalog number | Source |
|---|---|---|
| RPMI 1640 | 61870-036 | Gibco |
| Heat inactivated FBS | 10082-147 | Gibco |
| Penicillin-Streptomycin | P4458 | Sigma |
| Gentamycin | 15750037 | Gibco |
| Matrigel ™ Matrix | 354248 | Corning |

RPMI, Roswell Park Memorial Institute

TABLE 3
Reagents for PD analysis

| Reagent | Catalog number | Source |
|---|---|---|
| Pro-inflammatory Panel 1 kit V-Plex, | K151A0H-4 | MSD |
| Human IL6/IL10 | | |
| Diluent 2 | R51BB-3 | MSD |
| Streptavidin coated plates (MW96) | 15500 | ThermoScientific |
| Purified mouse anti-human BTK antibody | 611116 | BD Biosciences |
| Recombinant human BTK protein | PR5442A | LifeTechnologies |
| Goat anti-mouse HRP | 31444 | ThermoFisher |
| TMB Substrate | ES022 | MilliPore |
| H₂SO₄ | 4701 | J.T. Baker |
| Tween-20 | 170-6531 | BioRad |
| Bovine serum albumin | A9647 | Sigma |
| PBS | D8537 | Sigma |
| Round Bottom Plates | 353910 | Falcon |

### Cell Culture Methods

The human ABC-DLBCL cell line OCI-LY-0 was obtained from University Hospital Network, Ontario Cancer Institute. OCI-LY10 cells were maintained as suspension cells at 37° C. in a humidified atmosphere (5% C₂, 95% air), in RPMI-1640 medium, supplemented with m Fetal Bovine Serum (Heat Inactivated for 2 hours at 57° C.) containing 2 mM glutamine, 100 units/mL penicillin G sodium, 100 ag/mL streptomycin sulfate and 25 μg/mL gentamicin. Cells were passaged once a week and seeded at 0.2×10⁶ cells/mL in T225 culture flasks with medium change after 3-4 days. Each mouse received 1×10⁶ or 5×10⁶ OCI-LY10 cells in Dulbecco's phosphate buffered saline (DPBS) containing 50% Matrigel™ (BD Biosciences) in a total volume of 0.1 mL. Cells were implanted SC in the right flank using a 1 mL syringe and a 26-gauge needle. The day of tumor implantation was designated as Day 0.

### Study Designs

The doses, selected for Compound B anti-tumors efficacy, were based on single dose PK/PD data. Doses selected for Compound B and Compound A combination treatment were based on antitumor efficacy data obtained from single compound treatments. Study designs are summarized in Table 4 and described below.

TABLE 4
Study design and treatments

| | | | Mean tumor | | | Treatment groups |
|---|---|---|---|---|---|---|
| | | | volume at | | | Compound B (+ |
| | Tumor | | randomization | Treatment | Dosing | Compound A |
| Study | model | Study type | (mm³) | Duration | schedule | for combinations) |
| Study 1 | OCI-LY10 | PK/PD | 525 | Single | Single | 0, 1, 3, 10, 30, |
| | in NSG | | | dose | dose | 100 mg/kg, in |
| | mice | | | | | PEG400 + PVP- |
| | | | | | | VA, 5 mL/kg; |
| | | | | | | (n = 15) |
| Study 2 | OCI-LY10 | Monotherapy | 207 | 3 weeks | QD | 0, 10, 30, 100 |
| | in NSG | efficacy | | | BID | mg/kg, 0, 5, 15 |
| | mice | | | | | and 50 mg/kg in |
| | | | | | | PEG400 + |
| | | | | | | PVPVA; |
| | | | | | | 5 mL/kg |
| | | | | | | (n = 10) |
| Study 3 | OCI-LY10 | Combination | 165 | 3 weeks | QD | 30 and 100 |
| | in NSG | Efficacy | | | | mg/kg |
| | mice | | | | BID | 10 and 30 mg/kg |
| | | | | | QD (+ | 0 and 30 mg/kg (+ |
| | | | | | BID) | 0, 10, and 30 |
| | | | | | | mg/kg) |
| | | | | | QD (+ | 100 mg/kg (+ 10 |
| | | | | | BID) | and 30 mg/kg) |
| | | | | | | in PEG400, 5 |
| | | | | | | mL/kg (3.33 |
| | | | | | | mL/kg) (n = 10) |
| Study 4 | OCI-LY10 | Combination | 158 | 3 weeks | BID | 15 and 30 mg/kg |
| | in NSG | Efficacy | | | BID | 10 and 30 mg/kg |
| | mice | | | | BID (+ | 0 and 15 mg/kg (+ |
| | | | | | BID) | 0, 10, and 30 |
| | | | | | | mg/kg) |
| | | | | | BID (+ | 30 mg/kg (+ 10 |
| | | | | | BID) | and 30 mg/kg) in |
| | | | | | | PEG400, 2.5 |
| | | | | | | mL/kg (3.33 |
| | | | | | | mL/kg) (n = 10) |

PEG400, polyethylene glycol 400;
PK, pharmacokinetic;
PD, pharmacodynamic;
PVP-VA64, N-vinylpyrrolidone and vinyl acetate 64;
BID, bis in die (twice daily);
QD, quaque die (once daily).

In Study 1, 5×10⁶ OCI-LY10 cells in PBS containing 50% Matrigel™ were injected subcutaneously (SC) into the right hind flank of female NSG mice. Tumor growth was measured over time and once tumors reached volumes of approximately 525 mm³, mice were randomized in groups of 15 and treated orally with a single dose of Compound B according to treatment schedule (see Table 4). Blood was collected serially by mandibular vein sampling from 5 animals per time point per group to determine circulating compound concentration and IL10 levels at 2, 4, 8, 12, 16, and 24 hours post single dose. Blood was harvested in EDTA and plasma was obtained via centrifugation at 3000 rpm for 10 minutes. In addition, tumor samples were harvested from 5 animals per timepoint per treatment group for BTK occupancy studies at 4, 12, and 24 hours post single dose.

In efficacy study, Study 2, NSG female animals were injected SC into the right find flank with 5×10⁶ OCI-LY10 cells in PBS containing 50% Matrigel™. On day 32, mice were randomized based on tumor volume (mean tumor volume of 207 mm³) and treated orally once or twice daily with BTK inhibitor Compound B according to treatment schedule above (see Table 4) for 21 days. After the last dose on Day 53, blood was collected serially via submandibular vein sampling from 5 animals per time point per treatment group at 2, 4, 12, and 24 hours after dosing. Blood was collected in EDTA and plasma was obtained via centrifugation at 3000 rpm for 10 minutes. Samples were snap frozen and stored at −800° C. for possible future analyses.

Following early animal dropouts in Study 2 due to tumor progression (metastasis and hindlimb paralysis), NSG female mice in Study 3 and Study 4 were injected SC into the right flank with a lower cell number (1×10⁶ OCI-LY10 cells) in PBS containing 50% Matrigel™. On day 35 (Study 3) or day 32 (Study 4), mice were randomized based on tumor volume (see Table 4) and dosed orally QD day with Compound B and BID with Compound A (Study 3) or BID for both compounds (Study 4) with various dose concentrations according to treatment schedule above (see Table 4) for a period of 21 days.

### Animal Monitoring

Due to known tolerability issues with PEG400 and PVP-VA vehicles (see Hermansky et al., Food Chem. Toxicol. 1995; 33:139-149), body weights of all animals were followed daily for the first five days of treatment in efficacy studies. Subsequently animal body weight and tumor volume was monitored two to three times per week. Animals were monitored daily for clinical signs related to either compound toxicity or tumor burden (i.e., hind limb paralysis, lethargy, dyspnea, etc.). When individual animals exhibited negative clinical signs, reached a loss of >20% body weight as compared with initial body weight, or approached a maximum tumor volume endpoint of 2,000 mm³, they were removed from the study and humanely euthanized.

### PD Methods

NF-κB signaling regulates the secretion of multiple cytokines, including interleukin-10 (IL-10). Both BTK and MALT1 inhibition effect NF-kB signaling resulting in decreased IL-10 transcription and secretion. Circulating human IL-10 cytokine levels were measured in the serum of OCI-LY10 ABC-DLBCL tumor bearing NSG mice, using a Mesoscale Discovery assay (MSD). 25 μL of mouse serum was transferred to an MSD plate (V-Plex Proinflammation Panel 1 [human] kit) and incubated together with 25 μl diluent 2 (MSD; R51BB-3) for 2 hours at RT followed by a 2-hour incubation with IL-6/-10 antibody solution. Plates were read on a SECTOR imager.

Compound B is a covalent orally bioavailable inhibitor binding BTK irreversibly, allowing us to evaluate the duration of signaling shutdown and occupancy of BTK protein after compound administration considering compound binding to BTK as well as synthesis rate of new BTK protein. Target engagement was determined by measuring the amount of free BTK protein in OCI-LY10 DLBCL tumor lysates of mice treated with various dosing concentrations of Compound B using a BTK occupancy assay (ELISA assay format). A covalent BTK inhibitor probe, chemically linked to biotin (CNX-500 Probe), was incubated in PBS/BT (PBS+1% BSA+0.05% tween-20) with tumor lysate for 1 hour at 28° C. Incubated BTK standards and samples were transferred to a streptavidin-coated 96-well plates and mixed while shaking for 30 minutes at RT. The BTK-antibody was then added and incubated overnight at 4° C. After washing, goat anti-mouse-HRP was added and incubated for 1 hour at RT. The ELISA was developed with addition of tetramethyl benzidine (TMB) followed by sulfuric acid (stop solution) and read at optical density 450 nm on Envision device.

### Calculations

Body weight changes of individual mice were calculated using the formula: ([W−W0]/W0)×100, where “W” represents mean body weight on a particular day, and “W0” represents body weight at initiation of treatment. Body weight was graphed as mean body weight change±SEM. Toxicity was defined as ≥20% of mice in a given group demonstrating ≥20% body weight loss and/or death.

Tumor volume in SC models was calculated using the formula: tumor volume (mm³)=(D×d²/2); where “D” represents the larger diameter and “d” the smaller diameter of the tumor as determined by calliper measurements. Tumor volume data was graphed as the mean tumor volume f SEM.

The percent ΔTGI was defined as the difference between mean tumor burden of the treatment and control groups, calculated using the following formula: ([(TVc−TVc0)−(TVt−TVt0)]/(TVc−TVc0))×100, where “TVc” is the mean tumor burden of a given control group, “TVc0” is the mean initial tumor burden of a given control group, “TVt” is the mean tumor burden of the treatment group, and “TVt0” is the mean initial tumor burden of the treatment group. The percent tumor growth inhibition (TGI) was defined as the difference between mean tumor volumes of the treated and control groups, calculated as: ((TVc−TVt)/TVc)×100 where “TVc” is the mean tumor volume of the control group and “TVt” is the mean tumor volume of the treatment group. As defined by National Cancer Institute (NCI) criteria, ≥60% TGI is considered biologically significant. Johnson et al., Br J Cancer. 2001; 84(10):1424-1431.

Tumor regression was calculated when mean tumor burden in treated group was smaller than the tumor burden at start of treatment of the same treated group. The % Tumor Regression (TR), quantified to reflect the treatment-related reduction of tumor volume as compared to baseline independent of the control group, was calculated using the following formula: % TR=(1-mean (TVti/TVt0i))×100 where “TVti” is the tumor burden of individual animals in a treatment group, and “TVt0i” is the initial tumor burden of the animal.

A CR for SC tumor models was defined as complete tumor regression, with no palpable tumor on the day of analysis.

### Data Analysis

Tumor volume and body weight data were graphed using Prism software (GraphPad version 8). Statistical significance for most studies was evaluated for Compound B and Compound A-treated groups compared with vehicle-treated controls on the last day of the study when ⅔ or more mice remained in each group. Differences between groups were considered significant when p≤0.05.

Statistical significance for animal tumor volume and body weight for all SC tumor studies was calculated using the linear mixed-effects (LME) analysis in R software version 3.4.2 (using an internally developed Shiny application version 4.0), with treatment and time as fixed effects and animal as random effect. Pinheiro J, Bates D. Mixed-effects models in S and S-Plus; Heidelberg, Germany: Springer, 2000. Logarithmic transformation (base 10) was performed if individual longitudinal response trajectories were not linear. The information derived from this model was used to make pairwise treatment comparisons of animal body weights or tumor volumes to that of the control group or between all the treatment groups. Drug combination data was analyzed using the Bliss independence model. In this method, observed drug combination response is compared with predicted drug combination response obtained based on the assumption that there is no effect of drug to drug interactions. Combinations are declared synergistic when observed responses are greater than predicted responses.

### Results

### Body Weight

Compound B and Compound A are formulated in PEG400 or PEG400/PVP-VA64 (Study 1 and Study 2), which is known to cause diarrhea in rodents. Hermansky et al., Food Chem. Toxicol. 1995; 33:139-149. Overall, Compound B and Compound A were well tolerated in NSG mice at all dose levels tested (up to 50 mg/kg BID or 100 mg/kg QD for Compound B and 30 mg/kg BID for Compound A), with no individual animals reaching the maximum weight loss endpoint of 20% in Study 2.

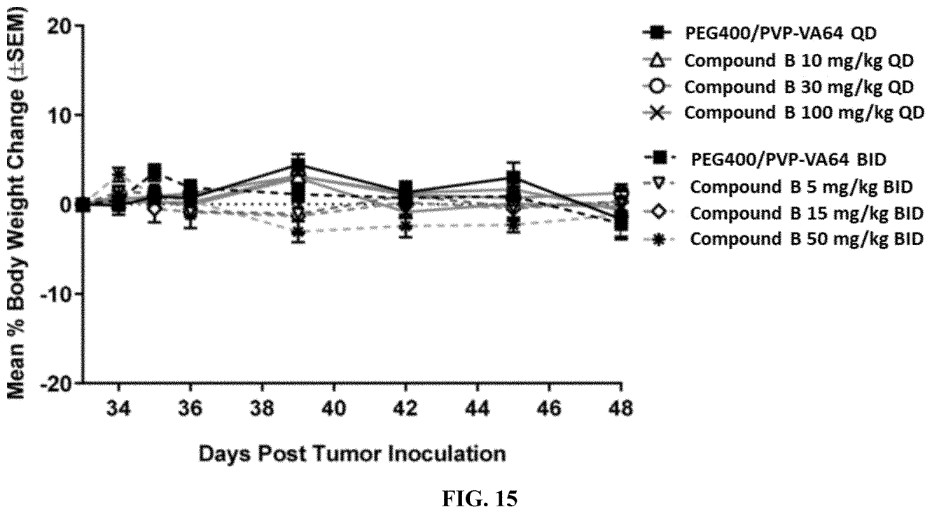

In efficacy study, Study 2, no body weight loss was observed during 3 weeks of treatment with Compound B (FIG. 15). Multiple animals were removed throughout the study from the vehicle, QD, and lower-dose BID Compound B-treated groups due to tumor progression; either reaching maximum allowed tumor volume endpoint or exhibiting clinical signs of tumor metastasis (hindlimb paralysis).

By 17 days into the 21-day treatment period, half of the animals in the vehicle QD control, 4 mice in the vehicle BID control, two animals in 10 mg/kg of Compound B QD dosing and a mouse in 100 mg/kg of Compound B QD dosing groups had succumbed to disease burden.

By 21 days of treatment, 7/10 animals were removed from the vehicle (QD) and 10 mg/kg (Compound B QD) treated group, and 9/10 mice from vehicle (BID) control group were removed from the study due to tumor progression. Interestingly, only 2/10, 3/10, 4/10, and 1/10 mice were removed from the 30 mg/kg QD, 100 mg/kg QD, 5 mg/kg BID, and 15 mg/kg BID of Compound B-treated groups, respectively. This indicated that BTK inhibition protected mice from tumor progression, including metastases.

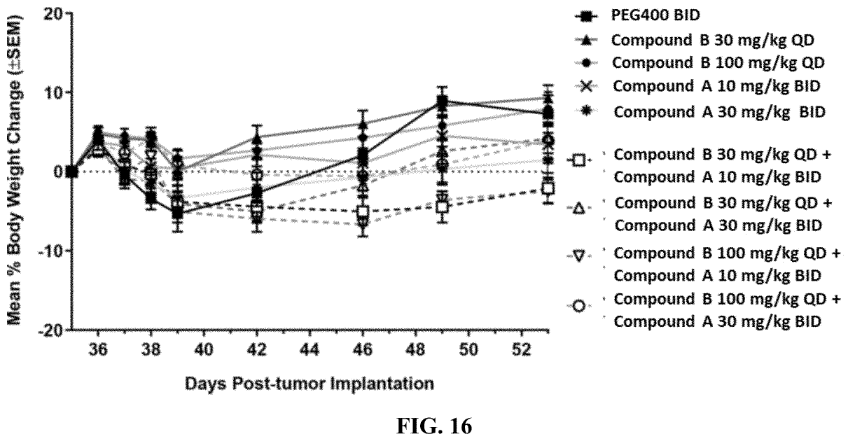

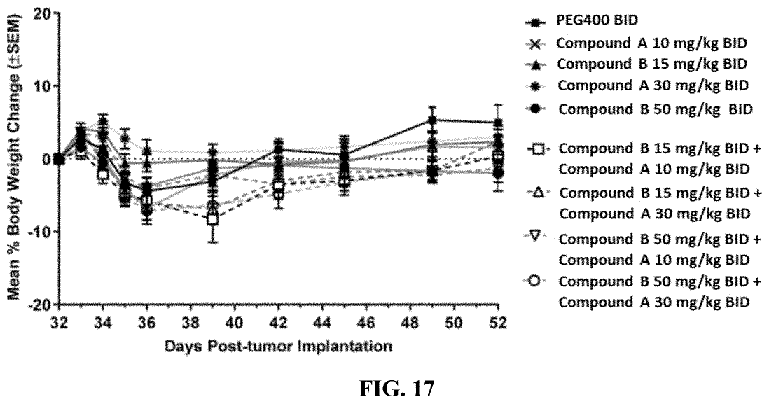

In combination efficacy studies Study 3 (FIG. 16) and Study 4 (FIG. 17), although toxic body weight loss was not observed, there were individual animals removed due to negative clinical signs, excessive body weight loss, or sporadic deaths. In addition, some low level, transient body weight loss was also observed during the 3 weeks of treatment with the vehicle control, Compound B, Compound A, or combinations. These observations were made at similar frequencies across the vehicle, monotherapy, and combination-treated groups, suggesting that frequent dosing/handling (3-4 times daily) plus vehicles that are known to cause diarrhea may have contributed.

Only one animal reached the maximum allowed body weight loss of 20% in Study 3, which was on day 56 post-tumor implantation, and after 3 weeks of treatment with 30 mg/kg QD of Compound B+10 mg/kg BID of Compound A.

Some individual animals from Study 3, were found dead or taken off study due to negative clinical signs: two animals in vehicle, one animal in 30 mg/kg QD of Compound B, one animal in 30 mg/kg BID of Compound A, one animal in 30 mg/kg QD of Compound B+10 mg/kg BID of Compound A, and one mouse in 100 mg/kg QD of Compound B+30 mg/kg BID of Compound A dosed groups. Multiple animals were also removed throughout the study from the QD vehicle and 1 animal in 30 mg/kg QD of Compound B-treated group due to clinical signs of tumor progression.

In Study 4, four animals were removed from the study due to reaching the maximum allowed body weight loss of 20%, with one each in 15 mg/kg of Compound B, 15 mg/kg of Compound B+10 mg/kg of Compound A and 15 mg/kg of Compound B+30 mg/kg of Compound A dosing and vehicle treated groups. One mouse in each group showed these adverse effects, indicating that these events were not compound or dose related, but rather PEG400 vehicle toxicity or excessive handling (4 oral gavages/day).

One mouse in the vehicle, one animal in 50 mg/kg of Compound B, four animals in 10 mg/kg of Compound A, two animals in 15 mg/kg of Compound B+10 mg/kg of Compound A and 1 animal in 15 mg/kg of Compound B+30 mg/kg of Compound A dosing groups died during the study.

### Efficacy

The antitumor efficacy of the BTK inhibitor alone and in combination with MALT1 inhibitor was assessed in mice bearing established SC OCI-LY10 human CD79b mutant DLBCL xenografts in female NSG mice.

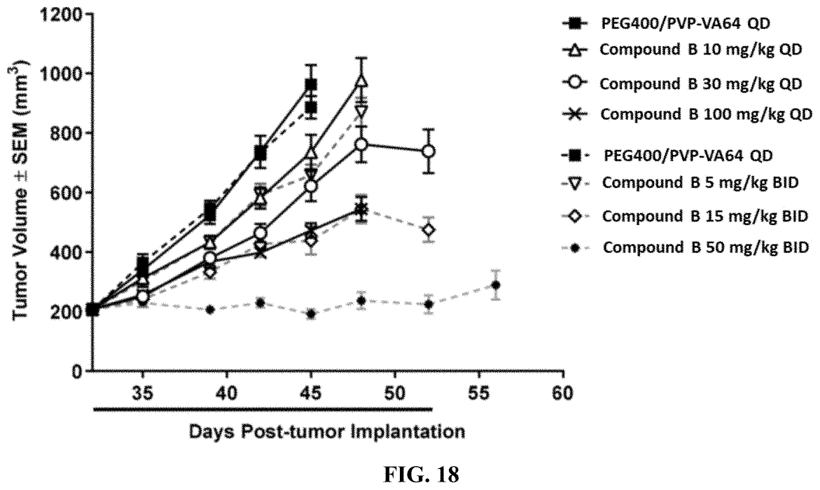

In Study 2, the antitumor efficacy of Compound B was evaluated as monotherapy in mice bearing OCI-LY10 xenografts, dosed either once (QD) or twice (BID) a day. Analysis of tumor growth inhibition was performed 14 days into the 21-day treatment period (Day 45) as that was the last day when ⅔ of the vehicle controls remained on the study. Compound B induced low-level tumor growth inhibition in the OCI-LY10 model at all dose levels. Treatment with 10, 30, and 100 mg/kg of Compound B administered QD inhibited tumor growth by 24%, 35%, and 51% TGI (30, 45, and 65% ΔTGI) respectively, as compared with vehicle treated control mice (p<0.05). BID treatments with 5, 15, and 50 mg/kg of the BTK inhibitor Compound B elicited slightly more pronounced tumor growth inhibition with 26%, 51%, and 78% TGI (34, 66, and 102% ΔTGI) (p<0.05), respectively, as compared to mice treated with vehicle control (FIG. 18). Overall, only the 50 mg/kg BID of Compound B treated group met the minimum NCI threshold criteria for biological significance (≥60/TGI). Johnson et al., Br J Cancer. 2001; 84(10):1424-1431.

When administered in combination with MALT1 inhibitor (Compound A), either QD or BID BTK inhibitor Compound B provided an enhanced antitumor benefit over either monotherapy that was additive/nearly additive at all dose levels tested, with partial tumor regressions observed at higher dose level combinations (Studies 3 and 4). The most pronounced antitumor efficacy and tumor regressions were observed with 50 mg/kg of Compound B BID in combination with either 10 or 30 mg/kg of Compound A.

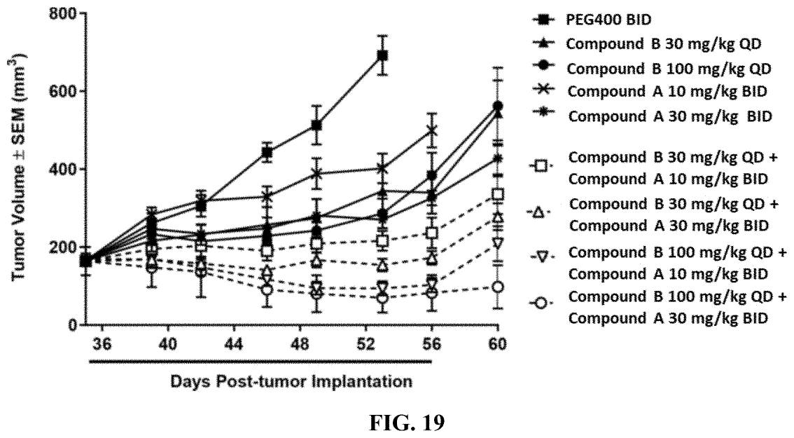

In Study 3, analysis of antitumor activity was performed 19 days into the planned 21-day treatment (day 53) when >⅔ of animals were still present in all treatment groups. Consistent with Study 2, in Study 3, 30 mg/kg of the BTK inhibitor Compound B given QD elicited 50% TGI (65% ΔTGI), and the higher 100 mg/kg QD dose level of Compound B closely approximated a biologically significant antitumor efficacy of 59% TGI (77% ΔTGI) as compared to vehicle-treated controls (FIG. 19). Similarly, MALT1 inhibitor Compound A monotherapy produced 42% TGI (54% ΔTGI) at 10 mg/kg BID, and 61% TGI (79% ΔTGI) at 30 mg/kg BID, as compared to the vehicle group, which was considered biologically significant. Efficacy was comparable to that observed previously with the MALT1-inhibitor (Compound A).

When Compound A (10 mg/kg BID) was given in combination with Compound B (30 mg/kg QD), enhanced antitumor activity of 69% TGI (90% ΔTGI) was observed (FIG. 19). Moreover, tumor stasis was achieved with TGI of 78% (103% ΔTGI) when BTK inhibitor Compound B (30 mg/kg QD) was combined with 30 mg/kg BID of the MALT1 inhibitor Compound A.

At the higher QD regimen of 100 mg/kg of Compound B combination with either 10 or 30 mg/kg BID treatment of the MALT1 inhibitor Compound A elicited 86% TGI (113% ΔTGI) and 90% TGI (118% ΔTGI), respectively. Furthermore, regression with TR values of 43% and 57%, respectively were observed in the 100 mg/kg of Compound B combination with either 10 or 30 mg/kg of Compound A as compared to initial tumor burden (FIG. 19).

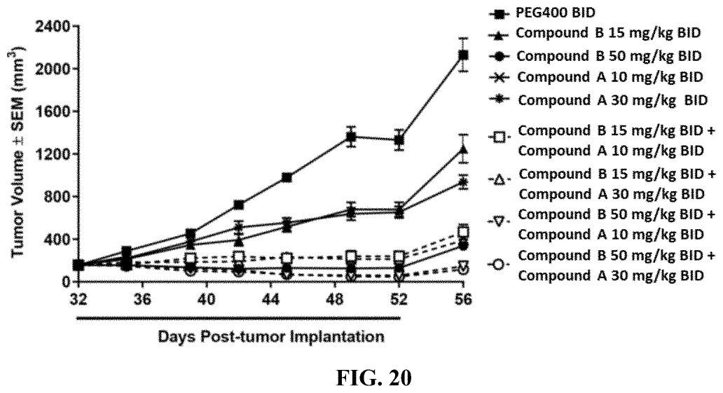

In Study 4, analysis of antitumor activity was performed at completion of a 21-day treatment (day 52) when >⅔ of animals were still present in all of the treatment groups, except for the low MALT1 inhibitor 10 mg/kg dosing group, which had fewer remaining. However, antitumor efficacy for the 10 mg/kg group was comparable with results from Study 3 and study for the MALT1 inhibitor (data not shown). Consistent with Study 2, 15 mg/kg of the BTK inhibitor Compound B given BID elicited 49% TGI (56% ΔTGI), and the higher 50 mg/kg BID dose level of Compound B a biologically significant 90% TGI (102% ΔTGI) as compared to vehicle-treated controls (FIG. 20). MALT1 inhibitor Compound A monotherapy produced 39% TGI (44% ΔTGI) at 10 mg/kg BID, and 51% TGI (58% ΔTGI) at 30 mg/kg BID, as compared to the vehicle group, which did not reach biological significance as compare to vehicle-treated mice.

When Compound A (10 mg/kg BID) was given in combination with Compound B (15 mg/kg BID), enhanced antitumor activity of 82% TGI (93% ΔTGI) was observed (P<0.05) (FIG. 20). Similar antitumor activity was also achieved with 15 mg/kg BID of the BTK inhibitor Compound B in combination with 30 mg/kg BID of the MALT1 inhibitor Compound A, with 84% TGI (95% ΔTGI) as compared to the vehicle control group (p<0.05).

At the higher BID regimen of 50 mg/kg of Compound B combination with either 10 or 30 mg/kg BID treatment of the MALT1 inhibitor Compound A elicited 95% TGI (108% ΔTGI) and 96% TGI (109% ΔTGI), respectively (FIG. 20). Partial TR was observed with both the lower (10 mg/kg) and higher 30 mg/kg MALT1 combinations with 50 mg/kg Compound B, with 59% and 71% TR (p<0.05), respectively, as compared to initial tumor burden.

### PD Effects of the BTK Inhibitor

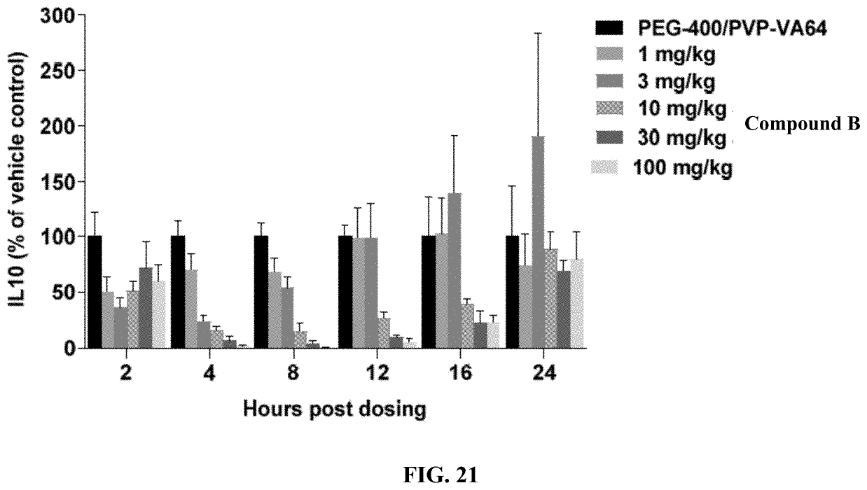

To evaluate the effect of Compound B on NFκB signaling, circulating human IL-10 levels in serum of NSG mice implanted with OCI-LY10 DLBCL tumors treated with 0, 1, 3, 10, 30, and 100 mg/kg of Compound B at 2, 4, 8, 12, 16, and 24 hours after single dose administration were analyzed. Human IL-10 levels dropped to around 50% of vehicle control 2 hours after dosing, lowering even further after 4 hours to below 20%, 10%, and 5% of vehicle control IL-10 levels in 10 mg/kg, 30 mg/kg, and 100 mg/kg of Compound B treatment groups, respectively, and remaining low up to 12 hours. Some rebound to 23% of vehicle control levels for 30 mg/kg and 100 mg/kg and to 39% for the 10 mg/kg dosing group are observed 16 hours after compound administration, while IL-10 levels normalize after 24 hours (FIG. 21).

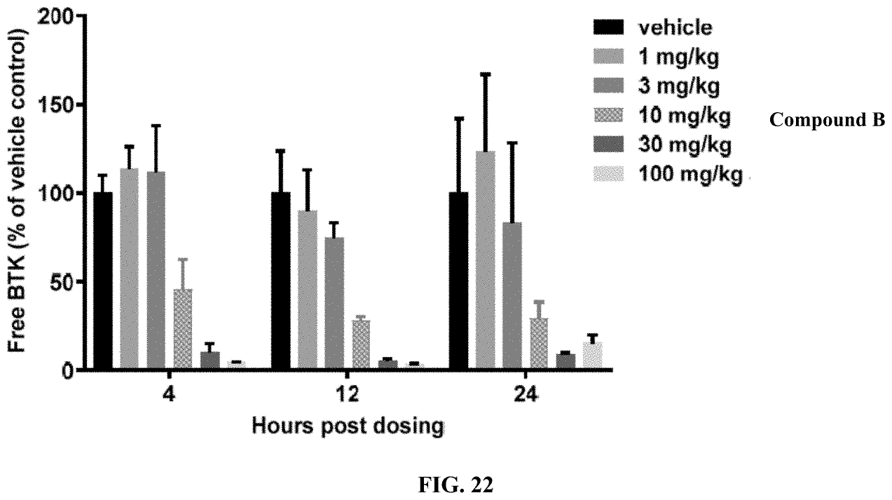

To evaluate the duration of signaling shutdown and occupancy of BTK protein after compound administration, the amount of free BTK protein in OCI-LY10 DLBCL tumor lysates harvested from Study 1 using an BTK occupancy assay was determined. No BTK occupancy was observed in OCI-LY10 DLBCL tumor lysates of animals dosed with 1 and 3 mg/kg of Compound B. However, 54%, 90%, and 95% BTK protein occupancy was observed 4 hours after Compound B dosing at dose levels of 10, 30, and 100 mg/kg, respectively. BTK protein occupancy levels remained high with 71%, 94%, and 96%, respectively, at 12 hours and 70%, 91%, and 85%, respectively after 24 hours (FIG. 22).

### Discussion

The PD and anti-tumor efficacy of Compound B was evaluated in a human ABC-DLBCL xenograft model in NSG mice, either as a monotherapy or in combination with the MALT1 inhibitor Compound A. Table 5 provides a brief summary of these studies.

TABLE 5
Summary of Efficacy for BTK inhibitor monotherapy and combination with MALT1 inhibitor

| | Species/ | Treatment | Animals per | |
|---|---|---|---|---|
| Study Type | Test System | Duration | Group (M/F) | Dose Groups and Key Results^{a} |
| OCI-LY10 PK/PD | NSG mice | Single Dose | 5 (F) | Dose-and time-dependent |
| (Study 1) | | | | decrease in circulating |
| | | | | human IL-10 cytokine |
| | | | | serum levels of mice treated |
| | | | | with Compound B at 1, 3, |
| | | | | 10, 30, or 100 mg/kg, with |
| | | | | maximal decreases observed |
| | | | | at doses ≥ 10 mg/kg from 4- |
| | | | | 8 hours, continued suppression |
| | | | | up to 12 hours, and returning |
| | | | | to baseline by 24 hours post- |
| | | | | single dose. |
| | | | | Dose-and time-dependent |
| | | | | decrease in unoccupied (free) |
| | | | | BTK in tumors from mice |
| | | | | treated with Compound B at |
| | | | | 1, 3, 10, 30, or 100 mg/kg, with |
| | | | | maximal decreases observed at |
| | | | | doses ≥ 30 mg/kg with sustained |
| | | | | suppression through 24 hours |
| | | | | post-single dose. |
| OCI-LY10 | NSG mice | 3 weeks | 10 (F) | 24% TGI (30% ΔTGI) at 10 |
| monotherapy | | | | mg/kg QD; 35% TGI (45% ΔTGI) |
| efficacy (Study 2) | | | | at 30 mg/kg QD; 51% |
| | | | | TGI (65% ΔTGI) at 100 mg/kg |
| | | | | QD; 26% TGI (34% ΔTGI) at |
| | | | | 5 mg/kg BID; 51% TGI |
| | | | | (66% ΔTGI) at 15 mg/kg BID; |
| | | | | 78% TGI (102% ΔTGI) at 50 |
| | | | | mg/kg BID; Compound B po |
| | | | | for 21 doses |
| OCI-LY10 | NSG mice | 3 weeks | 10 (F) | 50% TGI (65% ΔTGI) at 30 |
| combination | | | | mg/kg QD Compound B; 59% |
| efficacy (Study 3) | | | | TGI (77% ΔTGI) at 100 mg/kg |
| | | | | QD Compound B; 42% TGI |
| | | | | (54% ΔTGI) at 10 mg/kg BID |
| | | | | Compound A; 61% TGI |
| | | | | (79% ΔTGI) at 30 mg/kg BID |
| | | | | Compound A; 69% TGI |
| | | | | (90% ΔTGI) at 30 mg/kg QD |
| | | | | Compound B + 10 mg/kg BID |
| | | | | Compound A; 78% TGI |
| | | | | (103% ΔTGI) at 30 mg/kg |
| | | | | QD Compound B + 30 mg/kg |
| | | | | BID Compound A; 86% TGI |
| | | | | (113% ΔTGI) at 100 mg/kg QD |
| | | | | Compound B + 10 mg/kg BID |
| | | | | Compound A; 90% TGI |
| | | | | (118% ΔTGI) at 100 mg/kg QD |
| | | | | Compound B + 30 mg/kg BID |
| | | | | Compound A; po for 21 doses |
| OCI-LY10 | NSG mice | 3 weeks | 10 (F) | 49% TGI (56% ΔTGI) at 15 |
| combination | | | | mg/kg BID Compound B; 90% |
| efficacy (Study 4) | | | | TGI (102% ΔTGI) at 50 mg/kg |
| | | | | BID Compound B; 39% TGI |
| | | | | (44% ΔTGI) at 10 mg/kg BID |
| | | | | Compound A; 51% TGI |
| | | | | (58% ΔTGI) at 30 mg/kg BID |
| | | | | Compound A; 82% TGI |
| | | | | (93% ΔTGI) at 15 mg/kg BID |
| | | | | Compound B + 10 mg/kg BID |
| | | | | Compound A; 84% TGI |
| | | | | (95% ΔTGI) at 15 mg/kg BID |
| | | | | Compound B + 30 mg/kg BID |
| | | | | Compound A; 95% TGI |
| | | | | (108% ΔTGI) at 50 mg/kg BID |
| | | | | Compound B + 10 mg/kg BID |
| | | | | Compound A; 96% TGI |
| | | | | (109% ΔTGI) at 50 mg/kg |
| | | | | BID Compound B + 30 mg/kg |
| | | | | BID Compound A; po for 21 doses |

BTK, Bruton’s tyrosine kinase; M, male; F, female; NSG, non-obese diabetic severe combined immunodeficient gamma; ΔTGI, delta tumor growth inhibition (as compared to vehicle-treated control group); TGI, tumor growth inhibition (as compared to vehicle-treated control group); CR, complete response; qd, quaque die (once daily); PD, pharmacodynamic; PK, pharmacokinetic.
^{a}All p-values were ≤0.5 versus vehicle control except where noted as not significant (ns).

In the established subcutaneous (SC) OCI-LY10 DLBCL model (Study 2), Compound B induced statistically significant anti-tumor efficacy at all dose levels administered either daily (QD) or twice a day (BID). Tumor growth inhibition (TGI) of 24%, 35%, and 51% was observed when mice were treated with 10, 30, and 100 mg/kg QD, and 26%, 51%, and 78% TGI observed when treated with 5, 15, and 50 mg/kg BID, respectively, as compared to vehicle control-treated mice.

In the SC OCI-LY10 tumor model (Study 1), a single dose as low as 30 mg/kg of Compound B completely inhibited serum interleukin (IL)-10 secretion and displayed complete BTK occupancy in tumors, with effects lasting up to 24 hours post single dose.

When administered in combination with MALT1 inhibitor Compound A, either QD or BID BTK inhibitor Compound B provided an enhanced antitumor benefit over either monotherapy that was additive/nearly additive at all dose levels tested, with partial tumor regressions observed at higher dose level combinations (Studies 3 and 4). The most pronounced antitumor efficacy and tumor regressions were observed with 50 mg/kg of Compound B BID in combination with either 10 or 30 mg/kg of Compound A.

In the established SC OCI-LY10 DLBCL model, combined Compound B QD plus Compound A BID treatment induced statistically significant antitumor efficacy at all combination dose levels (p<0.05). When 30 mg/kg QD of Compound B was combined with 10 mg/kg BID of Compound A, enhanced TGI of 69% (90% ΔTGI) was observed (versus 50% TGI (65% ΔTGI) and 42% TGI (54% ΔTGI) for Compound B and Compound A monotherapies, respectively). Moreover, tumor stasis was achieved with a TGI of 78% (103% ΔTGI) when 30 mg/kg QD Compound B was combined with 30 mg/kg BID of Compound A (versus 61% TGI (79% ΔTGI) with Compound A monotherapy). At the higher 100 mg/kg QD dose level of Compound B monotherapy induced 59% TGI (76% ΔTGI) and combination with 10 or 30 mg/kg BID treatment of Compound A elicited 86% TGI (113% ΔTGI) and 90% TGI (118% ΔTGI), respectively, with 43% and 57%, tumor regressions (TR) observed.

Likewise, in Study 4, combined Compound B BID plus Compound A BID treatment induced statistically significant antitumor efficacy in all combination dose levels (p<0.05). TGI of 82% and 84% was observed with 15 mg/kg Compound B BID plus 10 mg/kg and 30 mg/kg Compound A BID, respectively (versus 49%, 39% and 51% TGI with 15 mg/kg Compound B BID, 10 mg/kg and 30 mg/kg Compound A BID monotherapies, respectively) as compared to vehicle treated animals. At the higher 50 mg/kg BID dose level of Compound B monotherapy induced 90% TGI (102% ΔTGI, 8% TR) and combination with 10 or 30 mg/kg Compound A BID treatment elicited 95% TGI (108% ΔTGI) and %% TGI (109% ΔTGI), respectively, with 59% and 71% TRs.

Although toxic body weight loss was not observed in these studies, there were individual animals removed due to negative clinical signs, excessive body weight loss, or sporadic deaths. In addition, some low level, transient, body weight loss was also observed during the 3 weeks of treatment with the vehicle control, Compound B, Compound A, or combinations. These observations were made at similar frequencies across the vehicle, monotherapy, and combination-treated groups, suggesting that frequent dosing/handling (3-4 times daily) plus vehicles that are known to cause diarrhea may have contributed. Taken together with the in vitro data demonstrating synergistic tumor cell killing shown in Examples 1 and 2, the studies in this Example provide support for clinical investigation of combination therapy with BTK inhibitor Compound B and the MALT1 inhibitor Compound A as a treatment for ABC-DLBCL and other B cell malignancies.

### Example 4: Combination of Compound A and Compound B—Results in Tumor Regressions in the ABC-like DLBCL Patient-Derived Xenograft LY2298

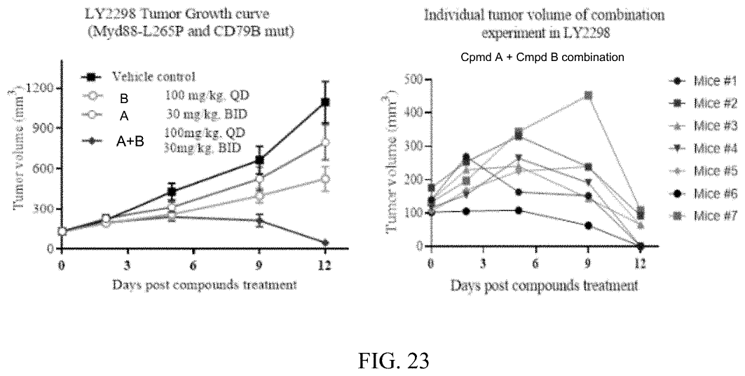

The in vivo therapeutic efficacy of Compound A and Compound B administered together was further evaluated in the B cell lymphoma patient derived xenograft (PDX) model LY2298 in female NOD/SCID mice. This tumor was derived from a patient with ABC-like DLBCL and has been genetically characterized to have mutations in CD79b, MYD88 and TP53. Compound A and Compound B were administrated orally together to LY2298 tumor bearing mice at 100 mg/kg QD and 30 mg/kg BID, respectively. At 12 days post treatment, compared with vehicle, the combination of Compound A and Compound B demonstrated significant in vivo efficacy with TGI of 108.7% (p<0.0001) from baseline. In contrast, Compound B 100 mg/kg QD or Compound A 30 mg/kg BID administered alone demonstrated TGI of 59.2% (p=0.3) and 30.9% (p=not significant), respectively, at equivalent doses. All 7 treated mice experienced tumor regression at 12 days posttreatment, while this was not observed in the monotherapy arms. The tumor growth curves and individual tumor volumes following treatment with Compound A and Compound B administered together are presented in FIG. 23. These results confirm that Compound A and Compound B are synergistic in the mouse PDX model of patient lymphoma. There was no weight loss >5% for any of the treated groups including the combination, indicating that the compounds were well tolerated.

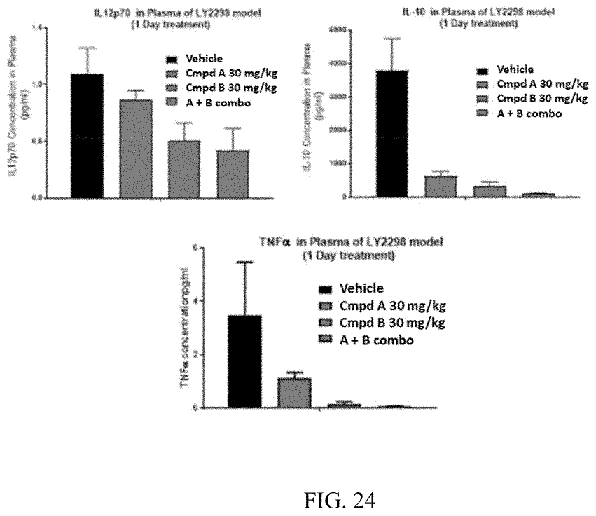

Cytokine secretion from serum samples of LY2298 tumor bearing mice was measured following Compound A and Compound B treatment. At 1 day posttreatment, compared with vehicle, monotherapy treatment with Compound A or Compound B significantly downregulated the serum secretion in LY2298 tumor bearing mice of 3 NF-κB-driven cytokines: interleukin (IL)10, tumor necrosis factor α (TNF α), and IL 12p70. Increased downregulation was observed when Compound A and Compound B were administered together (FIG. 24), reaching statistical significance for IL-10 relative to the monotherapy arms. Similarly, IL-10, TNF α, and IL 12p70 were also downregulated to the same or greater extent as the single agents after 12 days of therapy in the efficacy study (data not shown).

### Example 5: Drug-Drug Interaction Prediction Between Compound a and BTK Inhibitor

Physiological Based Pharmacokinetic (PBPK) modelling is an approach used to characterize drug disposition in a population. PBPK models are tools that simulate drug exposures to describe the PK (absorption, metabolism and excretion), predict potential drug-drug interactions (DDIs) and inform dosing strategies in virtual populations with accuracy. PBPK models can be used to extrapolate PK assessments beyond the study population and experimental conditions and help address a variety of clinical issues that may be encountered in the real-world.

A PBPK model of Compound A and Ibrutinib were developed (SimCYP v19) and were well verified with observed in vivo kinetics data after separate administration. Combined with the observed interaction in vitro data of Compound A, these models were used to evaluate a potential DDI after multiples doses of Compound A (once steady state concentrations are reached) on a single dose of Ibrutinib in a virtual population of 100 people. As shown in Table 6 below, the Cₘₐₓ and AUC values for Ibrutinib were predicted to increase when administered in combination with Compound A. At the dose of 420 mg Ibrutinib and 200 mg Compound A, the mean Cₘₐₓ of Ibrutinib is predicted to increase 43% and the AUC of Ibrutinib is predicted to increase 604, relative to Ibrutinib alone. Likewise, at the dose of 560 mg Ibrutinib and 300 mg of Compound A, the mean Cₘₐₓ of Ibrutinib is predicted to increase 50% and the AUC of Ibrutinib is predicted to increase 70%, relative to Ibrutinib alone.

TABLE 6

| | | AUC | Cmax | AUC | Cmax | AUC | Cmax |
|---|---|---|---|---|---|---|---|
| | | (ng/ml · h) | (ng/ml) | (ng/ml · h) | (ng/ml) | ratio | ratio |
| | | Alone | | MALT 200 mg | | | |
| Ibrutinib | Mean | 442 | 131 | 678 | 182 | 1.60 | 1.43 |
| 420 mg | 5^{th} perc | 168 | 41 | 285 | 59 | 1.27 | 1.20 |
| | 95^{th} perc | 883 | 246 | 1278 | 344 | 2.09 | 1.82 |
| Ibrutinib | Mean | 590 | 174 | 904 | 243 | 1.60 | 1.43 |
| 560 mg | 5^{th} perc | 224 | 54 | 381 | 79 | 1.27 | 1.20 |
| | 95^{th} perc | 1177 | 328 | 1704 | 459 | 2.09 | 1.82 |
| | | Alone | | MALT 300 mg | | | |
| Ibrutinib | Mean | 442 | 131 | 718 | 191 | 1.70 | 1.50 |
| 420 mg | 5^{th} perc | 168 | 41 | 305 | 62 | 1.34 | 1.24 |
| | 95^{th} perc | 883 | 246 | 1369 | 352 | 2.31 | 2.03 |
| Ibrutinib | Mean | 590 | 174 | 957 | 255 | 1.70 | 1.50 |
| 560 mg | 5^{th} perc | 224 | 54 | 406 | 82 | 1.34 | 1.24 |
| | 95^{th} perc | 1177 | 328 | 1826 | 469 | 2.31 | 2.03 |

Preliminary data from clinical studies closely mirrored the predicted modelling data. Data from preliminary studies (n=3-6) suggest that after dosing 420 mg Ibrutinib and 200 mg Compound A for 22 days, AUC (mean value) of Ibrutinib increased by 58%. Similarly, after dosing 420 mg Ibrutinib and 300 mg Compound A for 22 days, AUC (mean value) of Ibrutinib increased by 75%.

### Example 6: A Phase 1b, Open-Label Study of the Safety, Pharmacokinetics, and Pharmacodynamics of Compound B in Combination with Compound A in Participants with Non-Hodgkin Lymphoma and Chronic Lymphocytic Leukemia

Bruton's tyrosine kinase (BTK) is a cytoplasmic tyrosine kinase that plays a critical role in B cell activation via the B cell receptor (BCR) signaling pathway. BTK is important for normal B cell activation and the pathophysiology of B cell malignancies, and several BTK inhibitors have demonstrated clinical activity in non-Hodgkin lymphoma (NHL) and chronic lymphocytic leukemia (CLL). Compound B is an orally active, irreversible covalent BTK inhibitor. Given its BTK inhibitory potency, along with nonclinical data to date, JNJ-64264681 is likely to have similar anti-lymphoma activity to already approved BTK inhibitors.

Mucosa-associated lymphoid tissue lymphoma translocation protein 1 (MALT1) is a key mediator of the BCR signal transduction pathway positioned downstream of BTK. MALT1 plays a key role in activating the classical nuclear factor kappa-light-chain-enhancer of activated B cells (NF-κB) signaling pathway, which is important for B cell lymphoid malignancies, such as MCL, WM and diffuse large B cell lymphoma (DLBCL). As such, MALT1 has been shown to play a critical role in supporting tumor growth in different types of lymphoma, including activated B cell-like subtype of DLBCL (ABC-DLBCL). Compound A is an orally bioavailable, potent, and allosteric inhibitor of MALT1 that has demonstrated promising clinical activity and a favorable toxicity profile in a Phase 1 study. This study will evaluate Compound A in combination with Compound B in a first-in-human study of NHL and CLL.

### Objectives and Endpoints

The primary objectives of the study are to determine the safety (Part A and Part B) and the recommended Phase 2 doses (RP2Ds) (in Part A) of Compound A and Compound B when administered in combination in participants with B cell NHL and CLL.

The secondary objectives are to determine the safety of this combination in focused histologies/participant populations (Part B) when administered at the RP2D(s) determined in Part A. The secondary objectives are to assess the pharmacokinetics (PK) and pharmacodynamics (PD) of the study drugs (Part A and Part B), and to determine preliminary clinical activity of the combination in focused histologies/participant populations (Part B) when administered at the RP2D(s) determined in Part A.

The primary endpoint of the study is the type and severity of adverse events, including dose-limiting toxicities (DLTs). The study secondary endpoints include plasma concentration-time profiles, PK parameters, BTK receptor occupancy and cytokine and T cell profiling, overall response rate, time to first response, and duration of response.

### Study Design

This is an open-label, multicenter, Phase 1b study of Compound A and Compound B administered in combination in participants with B cell NHL and CLL who have relapsed or refractory disease that requires treatment. Compound A and Compound B will be administered orally in continuous 21-day cycles according to the dose escalation or cohort expansion strategy outlined below.

The study will be conducted in 2 parts: Part A of the study is designed to determine the RP2Ds of Compound A and Compound B when administered together in participants with B cell NHL and CLL. Dose escalation will begin with dose escalation Cohort 1, at the starting doses shown in Table 7. One or more RP2D(s) may be determined for further exploration in Part B.

TABLE 7
Proposed Dose Escalation Schedule of Compound A and Compound B

| | Compound A | | Compound B | |
|---|---|---|---|---|
| Cohort | Dose | Drug Product | Dose | Drug Product |
| 1 | 200 mg | 2 × 100 mg QD | 140 mg | 1 × 140 mg QD |
| 2 | 300 mg | 3 × 100 mg QD | 140 mg | 1 × 140 mg QD |
| 3 | 300 mg | 3 × 100 mg QD | 280 mg | 1 × 140 mg BID |
| 4 | 300 mg | 3 × 100 mg QD | 420 mg | 1 × 140 mg + 2 × 35 |
| | | | | mg BID |
| 5 | 300 mg | 3 × 100 mg QD | 560 mg | 2 × 140 mg BID |

Part B is designed to further assess the safety as well as preliminary clinical efficacy of the RP2D(s) of Compound A and Compound B when administered together in participants with specific subtypes of B cell NHL (eg, DLBCL, mantle cell lymphoma [MCL], follicular lymphoma [FL], mucosal-associated lymphoid tissue [MALT] lymphoma, marginal zone lymphoma [MZL], Waldenström macroglobulinemia [WM], small lymphocytic lymphoma [SLL]), or CLL. Approximately 20 participants per cohort may be enrolled at the RP2D(s) to confirm the data observed in Part A or based on expected activity in histologies of interest.

Dose escalation for ongoing participants will be guided by the dose escalation rules and will be decided by the Study Evaluation Team (SET) based on the review of safety, clinical activity, PK, PD, and other relevant data. The end of study is defined as the last scheduled study assessment for the last participant of the study.

### Number of Participants

A target of approximately 135 participants will be enrolled in this study. Because the number of cohorts to be opened will be informed and affected by the data herein and elsewhere, the final sample size may be different from 135.

### Treatment Groups and Duration

Participants will receive Compound A and Compound B administered together until disease progression, intolerable toxicity, withdrawal of consent, or the investigator determines that it is in the best interest of the participant to discontinue study drug treatment. Exceptions may be granted for participants continuing to derive clinical benefit.

### Efficacy Evaluations

The investigator will perform assessments to determine the response to therapy according to corresponding response assessment criteria appropriate for the histologies/populations.

### PK/PD Evaluations

Tumor, blood and plasma samples will be collected to evaluate the effect of first-dose, single and multiple doses of Compound A and Compound B when administered together. Samples will be collected at multiple timepoints and subjected to different PD assays such as biological activity of Compound A (measured by NF-kB assay) or BTK occupancy for Compound B in peripheral blood mononuclear cells (PBMCs) or tumor tissue.

### Safety Evaluations

The safety of Compound A and Compound B when administered together will be assessed by history, physical examinations, Eastern Cooperative Oncology Group (ECOG) performance status, clinical laboratory tests, vital signs, electrocardiograms (ECGs), and adverse event monitoring. Concomitant medication use will be recorded. The severity of adverse events will be assessed using National Cancer Institute Common Terminology Criteria for Adverse Events Version 5.0 (CTCAE V5.0).

### Statistical Methods

No formal statistical hypothesis testing will be conducted in this study. Dose escalation will follow a Bayesian Optimal Interval (BOIN) design. Data for dose escalation and cohort expansion will be primarily summarized using descriptive statistics.

### Preliminary Results

Twenty-four NHL and CLL patients were treated with 140 mg QD Compound B, in combination with 200 mg or 300 mg QD of Compound A. Response was evaluated for 23 patients, and 12 patients (52%) had partial or complete response (10 PRs, 2 CRs). In the 24 patients, 10 patients had CLL/SLL, and 9 were evaluated for response, and 6 patients (67%) showed partial response (PR). For majority of these patients, response was shown in first post-treatment disease evaluation at Cycle 3.

Another clinical trial is being conducted in the same patient population, in which Compound B is administered as a monotherapy. At the equivalent dose (140 mg QD) and higher dose (140 mg BID), none of the 7 treated patients had PR or CR. Two patients (1 MCL, 1 CLL) achieved PR soon after dose escalation to 280 mg BID at Cycles 7 and 9, respectively. These results, though with a small number of patients, suggested that 140 mg QD or 140 mg BID doses are likely to be suboptimal, when Compound B is used as a monotherapy, in contrast to the results from the combinational therapy with Compound A. In addition, when Compound A is administered as a single agent (50 mg-300 mg), no partial response or complete response was observed in 10 CLL/SLL patients. Therefore, the response seen in CLL/SLL patients in the combinational study is unlikely to attribute to either Compound A or Compound B alone.

### Illustrative Embodiments

Provided here are illustrative embodiments of the disclosed technology. These embodiments are illustrative only and do not limit the scope of the present disclosure or of the claims attached.

Embodiment 1. A method of treating a disorder or condition that is affected by the inhibition of MALT1 in a subject in need of treatment, comprising administering a therapeutically effective dose of a BTK inhibitor or a pharmaceutically acceptable salt form thereof ranging from about 25 to 1000 mg and a therapeutically effective dose ranging from about 25 to 1000 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A):

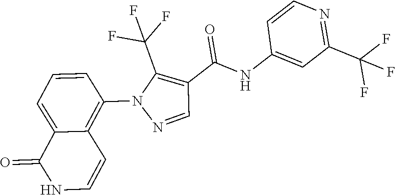 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof to said subject.

Embodiment 1a: A BTK inhibitor or pharmaceutically acceptable salt form thereof and 1 (1 oxo-1,2 dihydroisoquinolin-5 yl)-5 (trifluoromethyl)-N-[2 (trifluoromethyl)pyridin-4 yl]-1H-pyrazole-4 carboxamide (Compound A):

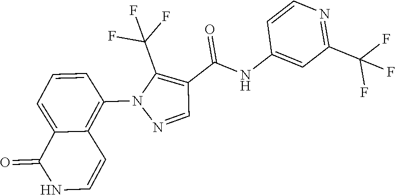 [SMILES: O=C(Nc1ccnc(C(F)(F)F)c1)c1cnn(-c2cccc3c(=O)[nH]ccc23)c1C(F)(F)F]

or a pharmaceutically acceptable salt form thereof, for use in treating a disorder or condition that is affected by the inhibition of MALT1 in a subject, comprising administering a therapeutically effective dose ranging from about 25 to 1000 mg of the BTK inhibitor or pharmaceutically acceptable salt form thereof, and a therapeutically effective dose ranging from about 25 to 1000 mg of Compound A or pharmaceutically acceptable salt form thereof to said subject.

Embodiment 2. The method of embodiment 1, wherein the subject is a human.

Embodiment 2a: The use according to embodiment 1a wherein the subject is a human.

Embodiment 3. The method of embodiment 2, wherein the therapeutically effective dose of Compound A is about 50 to 1000 mg, and the therapeutically effective dose of BTK inhibitor is about 50 to 1000 mg.

Embodiment 3a: The use according to embodiment 1a or 2a wherein the therapeutically effective dose of Compound A is selected from one of the about 50 to 1000 mg, and the therapeutically effective dose of BTK inhibitor is about 50 to 1000 mg.

Embodiment 4. The method of embodiment 2, wherein the therapeutically effective dose of Compound A is about 25 to 500 mg, and the therapeutically effective dose of BTK inhibitor is about 50 to 1000 mg.

Embodiment 4a: The use according to embodiment 1a or 2a wherein the therapeutically effective dose of Compound A is about 25 to 500 mg, and the therapeutically effective dose of BTK inhibitor is about 50 to 1000 mg.

Embodiment 5. The method of embodiment 2, wherein the therapeutically effective dose of Compound A is about 25 to 300 mg, and the therapeutically effective dose of BTK inhibitor is about 50 to 500 mg.

Embodiment 5a: The use according to embodiment 1a or 2a wherein the therapeutically effective dose of Compound A is about 25 to 300 mg, and the therapeutically effective dose of BTK inhibitor is about 50 to 500 mg.

Embodiment 6. The method of embodiment 2, wherein the therapeutically effective dose of Compound A is about 25 to 250 mg, and the therapeutically effective dose of BTK inhibitor is about 50 to 300 mg.

Embodiment 6a: The use according to embodiment 1a or 2a wherein the therapeutically effective dose of Compound A is about 25 to 250 mg, and the therapeutically effective dose of BTK inhibitor is about 50 to 300 mg.

Embodiment 7. The method of embodiment 2, wherein the therapeutically effective dose of Compound A is about 25 to 100 mg, and the therapeutically effective dose of BTK inhibitor is about 25 to 100 mg.

Embodiment 7a: The use according to embodiment 1a or 2a wherein the therapeutically effective dose of Compound A is about 25 to 100 mg, and the therapeutically effective dose of BTK inhibitor is about 25 to 100 mg.

Embodiment 8. The method of embodiment 2, wherein the therapeutically effective dose of Compound A is about 75 to 150 mg, and the therapeutically effective dose of BTK inhibitor is about 50 to 300 mg.

Embodiment 8a: The use according to embodiment 1a or 2a wherein the therapeutically effective dose of Compound A is about 75 to 150 mg, and the therapeutically effective dose of BTK inhibitor is about 50 to 300 mg.

Embodiment 9. The method of embodiment 2, wherein the therapeutically effective dose of Compound A is about 50 to 150 mg, and the therapeutically effective dose of BTK inhibitor is about 50 to 300 mg.

Embodiment 9a: The use according to embodiment 1a or 2a wherein the therapeutically effective dose of Compound A is about 50 to 150 mg, and the therapeutically effective dose of BTK inhibitor is about 50 to 300 mg.

Embodiment 10. The method of embodiment 2, wherein the therapeutically effective dose of Compound A is about 50 to 350 mg, and the therapeutically effective dose of BTK inhibitor is about 50 to 350 mg.

Embodiment 10a: The use according to embodiment 1a or 2a wherein the therapeutically effective dose of Compound A is about 50 to 350 mg, and the therapeutically effective dose of BTK inhibitor is about 50 to 350 mg.

Embodiment 11. The method of embodiment 2, wherein the therapeutically effective dose of Compound A is about 100 to 400 mg, and the therapeutically effective dose of BTK inhibitor is about 50 to 600 mg.

Embodiment 11a: The use according to embodiment 1a or 2a wherein the therapeutically effective dose of Compound A is about 100 to 400 mg, and the therapeutically effective dose of BTK inhibitor is about 50 to 600 mg.

Embodiment 12. The method of embodiment 2, wherein the therapeutically effective dose of Compound A is about 150 to 300 mg, and the therapeutically effective dose of BTK inhibitor is about 150 to 1000 mg.

Embodiment 12a: The use according to embodiment 1a or 2a wherein the therapeutically effective dose of Compound A is about 150 to 300 mg, and the therapeutically effective dose of BTK inhibitor is about 150 to 1000 mg.

Embodiment 13. The method of embodiment 2, wherein the therapeutically effective dose of Compound A is about 200 mg to 500 mg, and the therapeutically effective dose of BTK inhibitor is about 50 to 400 mg.

Embodiment 13a: The use according to embodiment 1a or 2a wherein the therapeutically effective dose of Compound A is about 200 mg, and the therapeutically effective dose of BTK inhibitor is about 50 to 400 mg.

Embodiment 14. The method of embodiment 2, wherein the therapeutically effective dose of Compound A is about 100 to 150 mg, and the therapeutically effective dose of BTK inhibitor is about 50 to 100 mg.

Embodiment 14a: The use according to embodiment 1a or 2a wherein the therapeutically effective dose of Compound A is about 100 to 150 mg, and the therapeutically effective dose of BTK inhibitor is about 50 to 100 mg.

Embodiment 15. The method of embodiment 2, wherein the therapeutically effective dose of Compound A is about 150 to 200 mg, and the therapeutically effective dose of BTK inhibitor is about 50 to 400 mg.

Embodiment 15a: The use according to embodiment 1a or 2a wherein the therapeutically effective dose of Compound A is about 150 to 200 mg, and the therapeutically effective dose of BTK inhibitor is about 50 to 400 mg.

Embodiment 16. The method of embodiment 2, wherein the therapeutically effective dose of Compound A is about 200 to 250 mg, and the therapeutically effective dose of BTK inhibitor is about 50 to 450 mg.

Embodiment 16a: The use according to embodiment 1a or 2a wherein the therapeutically effective dose of Compound A is about 200 to 250 mg, and the therapeutically effective dose of BTK inhibitor is about 50 to 450 mg.

Embodiment 17. The method of embodiment 2, wherein the therapeutically effective dose of Compound A is about 250 to 300 mg, and the therapeutically effective dose of BTK inhibitor is about 50 to 500 mg.

Embodiment 17a: The use according to embodiment 1a or 2a wherein the therapeutically effective dose of Compound A is about 250 to 300 mg, and the therapeutically effective dose of BTK inhibitor is about 50 to 500 mg.

Embodiment 18. The method of embodiment 2, wherein the therapeutically effective dose of Compound A is about 300 to 350 mg, and the therapeutically effective dose of BTK inhibitor is about 50 to 600 mg.

Embodiment 18a: The use according to embodiment 1a or 2a wherein the therapeutically effective dose of Compound A is about 300 to 350 mg, and the therapeutically effective dose of BTK inhibitor is about 50 to 600 mg.

Embodiment 19. The method of embodiment 2, wherein the therapeutically effective dose of Compound A is about 350 to 400 mg, and the therapeutically effective dose of BTK inhibitor is about 50 to 700 mg.

Embodiment 19a: The use according to embodiment 1a or 2a wherein the therapeutically effective dose of Compound A is about 350 to 400 mg, and the therapeutically effective dose of BTK inhibitor is about 50 to 700 mg.

Embodiment 20. The method of any one of embodiments 1-19, wherein the therapeutically effective dose of Compound A is administered twice daily for 7 days followed by once daily, and the therapeutically effective dose of the BTK inhibitor is administered twice daily.

Embodiment 20a: The use according to any one of embodiments 1a-19a, wherein the therapeutically effective dose of Compound A is administered twice daily for 7 days followed by once daily, and the therapeutically effective dose of the BTK inhibitor is administered twice daily.

Embodiment 21. The method of any one of embodiments 1-19, wherein the therapeutically effective dose of Compound A is administered twice daily for 7 days followed by once daily, and the therapeutically effective dose of the BTK inhibitor is administered once daily.

Embodiment 21a: The use according to any one of embodiments 1a-19a, wherein the therapeutically effective dose of Compound A is administered twice daily for 7 days followed by once daily, and the therapeutically effective dose of the BTK inhibitor is administered once daily.

Embodiment 22. The method of any one of embodiments 1-21, wherein the therapeutically effective dose of Compound A and therapeutically effective dose of BTK inhibitor is administered once daily.

Embodiment 22a: The use according to any one of embodiments 1a-21a, wherein the therapeutically effective dose of Compound A and therapeutically effective dose of BTK inhibitor is administered once daily.

Embodiment 23. The method of any one of embodiments 1-21, wherein the therapeutically effective dose of Compound A is administered once daily and therapeutically effective dose of BTK inhibitor is administered twice daily.

Embodiment 23a: The use according to any one of embodiments 1a-21a, wherein the therapeutically effective dose of Compound A is administered once daily and therapeutically effective dose of BTK inhibitor is administered twice daily.

Embodiment 24. The method of any one of embodiments 1-23, wherein said disorder or condition is cancer and/or immunological diseases.

Embodiment 24a: The use according to any one of embodiments 1a-23a, wherein said disorder or condition is cancer and/or immunological diseases.

Embodiment 25. The method of any one of embodiments 1-24, wherein said cancer is selected from the group consisting of lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 25a: The use according to any one of embodiments 1a-24a, wherein said disorder or condition is selected from the group consisting of lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

Embodiment 26. The method of any one of embodiments 1-24, wherein said immunological disease is selected from the group consisting of autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 26a. The use according to any one of embodiments 1a-24a, wherein said immunological disease is selected from the group consisting of autoimmune and inflammatory disorders, e.g. arthritis, rheumatoid arthritis (RA), psoriatic arthritis (PsA), inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjoergen's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including oedema, embolism, fibrosis, sarcoidosis, hypertension and emphysema, silicosis, respiratory failure, acute respiratory distress syndrome, BENTA disease, berylliosis, and polymyositis.

Embodiment 27. The method of any one of embodiments 1-24, wherein said disorder or condition is selected from the group consisting of non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

Embodiment 27a: The use according to any one of embodiments 1a-24a, wherein said disorder or condition is selected from the group consisting of non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

Embodiment 28. The method of any one of embodiments 1-24, wherein said disorder or condition is lymphoma.

Embodiment 28a: The use according to any one of embodiments 1a-24a, wherein said disorder or condition is lymphoma.

Embodiment 29. The method of any one of embodiments 1-24, wherein said disorder or condition is diffuse large B-cell lymphoma (DLBCL).

Embodiment 29a: The use according to any one of embodiments 1a-24a, wherein said disorder or condition is diffuse large B-cell lymphoma (DLBCL).

Embodiment 30. The method of any one of embodiments 1-24, wherein said disorder or condition is chronic lymphocytic leukemia (CLL).

Embodiment 30a: The use according to any one of embodiments 1a-24a, wherein said disorder or condition is chronic lymphocytic leukemia (CLL).

Embodiment 31. The method of any one of embodiments 1-24, wherein said disorder or condition small lymphocytic lymphoma (SLL).

Embodiment 31a: The use according to any one of embodiments 1a-24a, wherein said disorder or condition small lymphocytic lymphoma (SLL).

Embodiment 32. The method of any one of embodiments 1-31, wherein said subjects have received prior treatment with a Bruton tyrosine kinase inhibitor (BTKi).

Embodiment 32a. The use according to any one of embodiments 1a-31a, wherein said subjects have received prior treatment with a Bruton tyrosine kinase inhibitor (BTKi).

Embodiment 33. The method of any one of embodiment 1-28, wherein said lymphoma is MALT lymphoma.

Embodiment 33a. The use according to any one of embodiments 1a-28a, wherein said lymphoma is MALT lymphoma.

Embodiment 34. The method of any one of embodiments 1-24, wherein said disorder or condition is Waldenström macroglobulinemia (WM).

Embodiment 34a. The use according to any one of embodiments 1a-24a, wherein said disorder or condition is Waldenström macroglobulinemia (WM).

Embodiment 35. The method of any one of embodiments 1-34, wherein said disorder or condition is relapsed or refractory to prior treatment.

Embodiment 35a. The use according to any one of embodiments 1a-34a, wherein said disorder or condition is relapsed or refractory to prior treatment.

Embodiment 36. The method of any one of embodiments 1-35, wherein Compound A is used as a hydrate form thereof.

Embodiment 36a. The use according to any one of embodiments 1a-35a, wherein Compound A is used as a hydrate form thereof.

Embodiment 37. The method of any one of embodiments 1-35, wherein said subject is administered a pharmaceutical composition comprising Compound A or pharmaceutically acceptable salt form thereof and a pharmaceutically acceptable excipient, and a pharmaceutical composition comprising a BTK inhibitor or pharmaceutically acceptable salt form thereof and a pharmaceutically acceptable excipient.

Embodiment 37a. The use according to any one of embodiments 1a-35a, wherein said subject is administered a pharmaceutical composition comprising Compound A or pharmaceutically acceptable salt form thereof and a pharmaceutically acceptable excipient, and a pharmaceutical composition comprising a BTK inhibitor or pharmaceutically acceptable salt form thereof and a pharmaceutically acceptable excipient.

Embodiment 38. The method of any one of embodiments 1-37, wherein the BTK inhibitor is ibrutinib (1-[(3R)-3-[4-amino-3-(4-phenoxyphenyl)pyrazolo[3,4-d]pyrimidin-1-yl]piperidin-1-yl]prop-2-en-1-one).

Embodiment 38a. The use according to any one of embodiments 1a-37a, wherein the BTK inhibitor is ibrutinib (1-[(3R)-3-[4-amino-3-(4-phenoxyphenyl)pyrazolo[3,4-d]pyrimidin-1-yl]piperidin-1-yl]prop-2-en-1-one).

Embodiment 39. The method of any one of embodiments 1-37, wherein the BTK inhibitor is Roche BTKi RN486, acalabrutinib or zanubrutinib.

Embodiment 39a. The use according to any one of embodiments 1a-37a, wherein the BTK inhibitor is Roche BTKi RN486, acalabrutinib or zanubrutinib.

Embodiment 40. The method of any one of embodiments 1-37, wherein the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide.

Embodiment 40a. The use according to any one of embodiments 1a-37a, wherein the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide.

Embodiment 41. The method of any one of embodiments 1-37, wherein the method comprises from about 0.001 to about 200 mg of the BTK inhibitor per kg of the subject's body weight per day.

Embodiment 42. A method of treating diffuse large B-cell lymphoma (DLBCL) in a subject in need thereof comprising administering a therapeutically effective dose of Compound A or pharmaceutically acceptable salt form thereof and a therapeutically effective dose of BTK inhibitor or pharmaceutically acceptable salt form thereof to said subject.

Embodiment 42a. Compound A or a pharmaceutically acceptable salt form thereof and a BTK inhibitor or a pharmaceutically acceptable salt form thereof, for use in treating diffuse large B-cell lymphoma (DLBCL) in a subject, comprising administering a therapeutically effective dose of Compound A or a pharmaceutically acceptable salt form thereof and a therapeutically effective dose of BTK inhibitor or a pharmaceutically acceptable salt form thereof to said subject.

Embodiment 43. The method of embodiment 42, wherein the therapeutically effective dose of Compound A is about 50 to 500 mg, and the therapeutically effective dose of BTK inhibitor is about 50 to 500 mg.

Embodiment 43a. The use according to embodiment 42a, wherein the therapeutically effective dose of Compound A is about 50 to 500 mg, and the therapeutically effective dose of BTK inhibitor is about 50 to 500 mg.

Embodiment 44. A method of treating Waldenström macroglobulinemia in a subject in need thereof comprising: administering a therapeutically effective dose of Compound A or a pharmaceutically acceptable salt form thereof to said subject; and administering a therapeutically effective dose of BTK inhibitor or a pharmaceutically acceptable salt form thereof to said subject.

Embodiment 44a: Compound A or a pharmaceutically acceptable salt form thereof and a BTK inhibitor or a pharmaceutically acceptable salt form thereof, for use in treating Waldenström macroglobulinemia in a subject in need thereof comprising administering a therapeutically effective dose of Compound A or a pharmaceutically acceptable salt form thereof and a therapeutically effective dose of BTK inhibitor or a pharmaceutically acceptable salt form thereof to said subject.

Embodiment 45. The method of embodiment 44, wherein the therapeutically effective dose of Compound A is about 50 to 500 mg, and the therapeutically effective dose of BTK inhibitor is about 50 to 500 mg.

Embodiment 45a. The use according to embodiment 44a, wherein the therapeutically effective dose of Compound A is about 50 to 500 mg, and the therapeutically effective dose of BTK inhibitor is about 50 to 500 mg.

Embodiment 46. A method of treating mantle cell lymphoma in a subject in need thereof comprising administering a therapeutically effective dose of Compound A or a pharmaceutically acceptable salt form thereof and a therapeutically effective dose of BTK inhibitor or a pharmaceutically acceptable salt form thereof to said subject.

Embodiment 46a: Compound A or a pharmaceutically acceptable salt form thereof and a BTK inhibitor or a pharmaceutically acceptable salt form thereof, for use in treating mantle cell lymphoma in a subject in need thereof comprising administering a therapeutically effective dose of Compound A or a pharmaceutically acceptable salt form thereof and a therapeutically effective dose of BTK inhibitor or a pharmaceutically acceptable salt form thereof to said subject.

Embodiment 47. The method of embodiment 46, wherein the therapeutically effective dose of Compound A is about 50 to 500 mg, and the therapeutically effective dose of BTK inhibitor is about 50 to 500 mg.

Embodiment 47a. The use according to embodiment 46a, wherein the therapeutically effective dose of Compound A is about 50 to 500 mg, and the therapeutically effective dose of BTK inhibitor is about 50 to 500 mg.

Embodiment 48. A method of treating chronic lymphocytic leukemia in a subject in need thereof comprising: administering a therapeutically effective dose of Compound A or a pharmaceutically acceptable salt form thereof to said subject; and administering a therapeutically effective dose of BTK inhibitor or pharmaceutically acceptable salt form thereof to said subject.

Embodiment 48a: Compound A or a pharmaceutically acceptable salt form thereof and a BTK inhibitor or a pharmaceutically acceptable salt form thereof, for use in treating chronic lymphocytic leukemia in a subject in need thereof comprising administering a therapeutically effective dose of Compound A or a pharmaceutically acceptable salt form thereof and a therapeutically effective dose of BTK inhibitor or a pharmaceutically acceptable salt form thereof to said subject.

Embodiment 49. The method of embodiment 48, wherein the therapeutically effective dose of Compound A is about 50 to 500 mg, and the therapeutically effective dose of BTK inhibitor is about 50 to 500 mg.

Embodiment 49a. The use according to embodiment 48a, wherein the therapeutically effective dose of Compound A is about 50 to 500 mg, and the therapeutically effective dose of BTK inhibitor is about 50 to 500 mg.

Embodiment 50. The method of any one of embodiments 42 to 49, wherein the BTK inhibitor is ibrutinib (1-[(3R)-3-[4-amino-3-(4-phenoxyphenyl)pyrazolo[3,4.d]pyrimidin-1-yl]piperidin-1-yl]prop-2-en-1-one).

Embodiment 50a. The use according to any one of embodiments 42a to 49a, wherein the BTK inhibitor is ibrutinib (1-[(3R)-3-[4-amino-3-(4-phenoxyphenyl)pyrazolo[3,4-d]pyrimidin-1-yl]piperidin-1-yl]prop-2-en-1-one).

Embodiment 51. The method of any one of embodiments 42 to 49, wherein the BTK inhibitor is Roche BTKi RN486.

Embodiment 51a. The use according to any one of embodiments 42a to 49a, wherein the BTK inhibitor is Roche BTKi RN486.

Embodiment 52. The method of any one of embodiments 42 to 49, wherein the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide.

Embodiment 52a. The use according to any one of embodiments 42a to 49a, wherein the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide.

Embodiment 53. The method of any of embodiments 42-49, wherein the BTK inhibitor and/or Compound A are administered orally.

Embodiment 53a. The use according to any one of embodiments 42a-49a, wherein the BTK inhibitor and/or Compound A are administered orally.

Embodiment 54. The use according to any one of embodiments 1a-53a, wherein the BTK inhibitor is a compound of Formula (I):

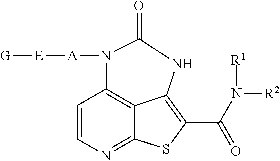

wherein

R¹ is H or C₁₋₆alkyl;

R² is selected from the group consisting of: C₀₋₆alk-cycloalkyl optionally substituted with 1, 2, or 3 substituents each independently selected from the group consisting of: NR⁸—C(O)—C(R³)═CR⁴(R⁵); NR⁶R⁷; OH; CN; oxo; O—C₁₋₆alkyl; halogen; C₁₋₆alkyl; C₁₋₆haloalkyl; C₁₋₆alk-OH; C₃₋₆cycloalkyl; C₁₋₆alkaryl; SO₂C₁₋₆alkyl; SO₂C₂₋₆alkenyl; NR⁸—C(O)—C₁₋₆alk-NR⁶R⁷; NR⁸—C(O)—C₁₋₆alkyl; NR⁸—C(O)—O—C₁₋₆alkyl; NR⁸—C(O)—C₃₋₆cycloalkyl; NR⁸—C(O)H; NR⁸—C(O)—C₃₋₆cycloalkyl; NR⁸—C(O)—C₁₋₆haloalkyl; NR⁸—C(O)-alkynyl; NR⁸—C(O)—C₆₋₁₀aryl; NR⁸—C(O)-heteroaryl; NR⁸—C(O)—C₁₋₆alk-CN; NR⁸—C(O)—C₁₋₆alk-OH; NR⁸—C(O)—C₁₋₆alk-SO₂—C₁₋₆alkyl; NR⁸—C(O)—C₁₋₆alk-NR⁶R⁷; NR⁸—C(O)—C₁₋₆alk-O—C₁₋₆alkyl wherein the C₁₋₆alk is optionally substituted with OH, OC₁₋₆alkyl, or NR⁶R⁷; and NR⁸—C(O)—C₀₋₆alk-heterocycloalkyl wherein the C₀₋₆alk is optionally substituted with oxo and the heterocycloalkyl is optionally substituted with C₁₋₆alkyl;

wherein R⁶ and R⁷ are each independently selected from the group consisting of: H; C₁₋₆alkyl; C₃₋₆cycloalkyl; C(O)H; and CN;

R³ is selected from the group consisting of: H, CN, halogen, C₁₋₆haloalkyl, and C₁₋₆alkyl;

R⁴ and R⁵ are each independently selected from the group consisting of: H; C₀₋₆alk-NR⁶R⁷; C₁₋₆alk-OH; C₀₋₆alk-C₃₋₆cycloalkyl optionally substituted with C₁₋₆alkyl; halogen; C₁₋₆alkyl; OC₁₋₆alkyl; C₁₋₆alk-O—C₁₋₆alkyl; C₁₋₆alk-NH—C₀₋₆alk-O—C₁₋₆alkyl; C₀₋₆alk-heterocycloalkyl optionally substituted with C(O)C₁₋₆alkyl or C₁₋₆alkyl; C₁₋₆alk-NHSO₂—C₁₋₆alkyl; C₁₋₆alk-SO₂—C₁₋₆alkyl; —NHC(O)—C₁₋₆alkyl; and -linker-PEG-Biotin;

R⁸ is H or C₁₋₆alkyl;

or R¹ and R², together with the nitrogen atom to which they are attached, form a pyrrolidinyl ring optionally substituted with NR⁶R⁷, wherein R⁶ and R⁷ are each independently selected from the group consisting of H; C₁₋₆alkyl; NR⁸—C(O)—C₁₋₆alkyl; and NR⁸—C(O)—C(R³)═CR⁴(R⁵), wherein R⁸ is H; R³ is H or CN; R⁴ is H; and R⁵ is H or cyclopropyl

A is selected from the group consisting of: a bond; pyridyl; phenyl; napthalenyl; pyrimidinyl; pyrazinyl; pyridazinyl; benzo[d][1,3]dioxolyl optionally substituted with halogen; benzothiophenyl; and pyrazolyl; wherein the A is optionally substituted with 1, 2, or 3 substituents each independently selected from the group consisting of: C₁₋₆alkyl; halogen; SF₅; OC₁₋₆alkyl; C(O)—C₁₋₆alkyl; and C₁₋₆haloalkyl;

E is selected from the group consisting of: O, a bond, C(O)—NH, CH₂, and CH₂—O;

G is selected from the group consisting of: H; C₃₋₆cycloalkyl; phenyl; thiophenyl; C₁₋₆alkyl; pyrimidinyl; pyridyl; pyridazinyl; benzofuranyl; C₁₋₆haloalkyl; heterocycloalkyl that contains an oxygen heteroatom; phenyl-CH₂—O-phenyl; C₁₋₆alk-O—C₁₋₆alkyl; NR⁶R⁷; SO₂C₁₋₆alkyl; and OH; wherein the phenyl; pyridyl; pyridazinyl; benzofuranyl; or thiophenyl is optionally substituted with 1, 2, or 3 substituents each independently selected from the group consisting of: halogen; C₁₋₆alkyl; C₁₋₆haloalkyl; OC₁₋₆haloalkyl; C₃₋₆cycloalkyl; OC₁₋₆alkyl; CN; OH; C₁₋₆alk-O—C₁₋₆alkyl; C(O)—NR⁶R⁷; and C(O)—C₁₋₆alkyl; and

stereoisomers and isotopic variants thereof; and pharmaceutically acceptable salts thereof.

A BTK inhibitor or a pharmaceutically acceptable salt form thereof, and Compound A or a pharmaceutically acceptable salt form thereof, for use in a method as described in any one of the embodiments described herein.

Use of a BTK inhibitor or a pharmaceutically acceptable salt form thereof, and Compound A or a pharmaceutically acceptable salt form thereof, for the manufacture of a medicament for a method of any one of the embodiments described herein.

A pharmaceutical product comprising Compound A and Compound B as a combined preparation for simultaneous, separate or sequential use in the treatment of non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

All embodiments described herein for methods of treating a disorder or condition, are also applicable for use in treating said disorder or condition.

All embodiments described herein for methods of treating a disorder or condition, are also applicable for use in a method of treating a disorder or condition.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention and that embodiments within the scope of these claims and their equivalents be covered thereby.

We claim:

1. A method of treating a disorder or condition that is affected by the inhibition of MALT1 in a subject in need of treatment, comprising administering a therapeutically effective dose ranging from about 25 to 1000 mg of a BTK inhibitor or pharmaceutically acceptable salt form thereof and a therapeutically effective dose ranging from about 25 to 1000 mg of 1-(1-oxo-1,2-dihydroisoquinolin-5-yl)-5-(trifluoromethyl)-N-[2-(trifluoromethyl)pyridin-4-yl]-1H-pyrazole-4-carboxamide (Compound A): 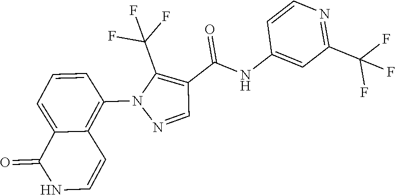 [SMILES: O=C(Nc1ccnc(C(F)(F)F)c1)c1cnn(-c2cccc3c(=O)[nH]ccc23)c1C(F)(F)F]
or a pharmaceutically acceptable salt form thereof to said subject.

2. The method of claim 1, wherein the therapeutically effective dose of Compound A is about 25 to 300 mg, and the therapeutically effective dose of BTK inhibitor is about 50 to 500 mg.

3. The method of claim 1, wherein the therapeutically effective dose of Compound A and the BTK inhibitor is administered one time a day.

4. The method of claim 1, wherein said disorder or condition is cancer or immunological diseases.

5. The method of claim 4, wherein the cancer is selected from the group consisting of lymphomas, leukemias, carcinomas, and sarcomas, e.g. non-Hodgkin's lymphoma (NHL), B-cell NHL, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), small lymphocytic lymphoma (SLL), Waldenström macroglobulinemia, lymphoblastic T cell leukemia, chronic myelogenous leukemia (CML), hairy-cell leukemia, acute lymphoblastic T cell leukemia, plasmacytoma, immunoblastic large cell leukemia, megakaryoblastic leukemia, acute megakaryocyte leukemia, promyelocytic leukemia, erythroleukemia, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head and neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, primary and secondary central nervous system lymphoma, transformed follicular lymphoma, diseases/cancer caused by API2-MALT1 fusion, and GIST (gastrointestinal stromal tumor).

6. The method of claim 4, wherein the cancer is selected from the group consisting of non-Hodgkin's lymphoma (NHL), diffuse large B-cell lymphoma (DLBCL), marginal zone lymphoma, mantle cell lymphoma (MCL), follicular lymphoma (FL), transformed follicular lymphoma, chronic lymphocytic leukemia, and Waldenström macroglobulinemia.

7. The method of claim 4, wherein the cancer is diffuse large B-cell lymphoma (DLBCL).

8. The method of claim 4, wherein the cancer is chronic lymphocytic leukemia (CLL).

9. The method of claim 4, wherein the cancer is small lymphocytic lymphoma (SLL).

10. The method of claim 4, wherein the cancer is Waldenström macroglobulinemia (WM).

11. The method of claim 1, wherein the BTK inhibitor is a compound of Formula (I): 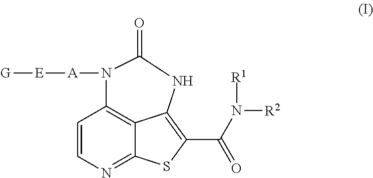
wherein
R¹ is H or C₁₋₆alkyl;
R² is selected from the group consisting of: C₀₋₆alk-cycloalkyl optionally substituted with 1, 2, or 3 substituents each independently selected from the group consisting of: NR⁸—C(O)—C(R³)═CR⁴(R⁵); NR⁶R⁷; OH; CN; oxo; O—C₁₋₆alkyl; halogen; C₁₋₆alkyl; C₁₋₆haloalkyl; C₁₋₆alk-OH; C₁₋₆cycloalkyl; C₁₋₆alkaryl; SO₂C₁₋₆alkyl; SO₂C₂₋₆alkenyl; NR⁸—C(O)—C₁₋₆alk-NR⁶R⁷; NR⁸—C(O)—C₁₋₆alkyl; NR⁸—C(O)—O—C₁₋₆alkyl; NR⁸—C(O)—C₃₋₆cycloalkyl; NR⁸—C(O)H; NR⁸—C(O)—C₃₋₆cycloalkyl; NR⁸—C(O)—C₁₋₆haloalkyl; NR⁸—C(O)-alkynyl; NR⁸—C(O)—C₆₋₁₀aryl; NR⁸—C(O)-heteroaryl; NR⁸—C(O)—C₁₋₆alk-CN; NR⁸—C(O)—C₁₋₆alk-OH; NR⁸—C(O)—C₁₋₆alk-SO₂—C₁₋₆alkyl; NR⁸—C(O)—C₁₋₆alk-NR⁶R⁷; NR⁸—C(O)—C₁₋₆alk-O—C₁₋₆alkyl wherein the C₁₋₆alk is optionally substituted with OH, OC₁₋₆alkyl, or NR⁶R⁷; and NR⁸—C(O)—C₀₋₆alk-heterocycloalkyl wherein the C₀₋₆alk is optionally substituted with oxo and the heterocycloalkyl is optionally substituted with C₁₋₆alkyl;
wherein R⁶ and R⁷ are each independently selected from the group consisting of: H; C₁₋₆alkyl; C₃₋₆cycloalkyl; C(O)H; and CN;
R³ is selected from the group consisting of: H, CN, halogen, C₁₋₆haloalkyl, and C₁₋₆alkyl;
R⁴ and R⁵ are each independently selected from the group consisting of: H; C₀₋₆alk-NR⁶R⁷; C₁₋₆alk-OH; C₀₋₆alk-C₃₋₆cycloalkyl optionally substituted with C₁₋₆alkyl; halogen; C₁₋₆alkyl; OC₁₋₆alkyl; C₁₋₆alk-O—C₁₋₆alkyl; C₁₋₆alk-NH—C₀₋₆alk-O—C₁₋₆alkyl; C₀₋₆alk-heterocycloalkyl optionally substituted with C(O)C₁₋₆alkyl or C₁₋₆alkyl; C₁₋₆alk-NHSO₂—C₁₋₆alkyl; C₁₋₆alk-SO₂—C₁₋₆alkyl; —NHC(O)—C₁₋₆alkyl; and -linker-PEG-Biotin;
R⁸ is H or C₁₋₆alkyl;
or R¹ and R², together with the nitrogen atom to which they are attached, form a pyrrolidinyl ring optionally substituted with NR⁶R⁷, wherein R⁶ and R⁷ are each independently selected from the group consisting of H; C₁₋₆alkyl; NR⁸—C(O)—C₁₋₆alkyl; and NR⁸—C(O)—C(R³)═CR⁴(R⁵), wherein R⁸ is H; R³ is H or CN; R⁴ is H; and R⁵ is H or cyclopropyl
A is selected from the group consisting of: a bond; pyridyl; phenyl; napthalenyl; pyrimidinyl; pyrazinyl; pyridazinyl; benzo[d][1,3]dioxolyl optionally substituted with halogen; benzothiophenyl; and pyrazolyl; wherein the A is optionally substituted with 1, 2, or 3 substituents each independently selected from the group consisting of: C₁₋₆alkyl; halogen; SF₅; OC₁₋₆alkyl; C(O)—C₁₋₆alkyl; and C₁₋₆haloalkyl;
E is selected from the group consisting of: O, a bond, C(O)—NH, CH₂, and CH₂—O;
G is selected from the group consisting of: H; C₃₋₆cycloalkyl; phenyl; thiophenyl; C₁₋₆alkyl; pyrimidinyl; pyridyl; pyridazinyl; benzofuranyl; C₁₋₆haloalkyl; heterocycloalkyl that contains an oxygen heteroatom; phenyl-CH₂—O-phenyl; C₁₋₆alk-O—C₁₋₆alkyl; NR⁶R⁷; SO₂C₁₋₆alkyl; and OH; wherein the phenyl; pyridyl; pyridazinyl; benzofuranyl; or thiophenyl is optionally substituted with 1, 2, or 3 substituents each independently selected from the group consisting of: halogen; C₁₋₆alkyl; C₁₋₆haloalkyl; OC₁₋₆haloalkyl; C₃₋₆cycloalkyl; OC₁₋₆alkyl; CN; OH; C₁₋₆alk-O—C₁₋₆alkyl; C(O)—NR⁶R⁷; and C(O)—C₁₋₆alkyl; and
stereoisomers and isotopic variants thereof; and pharmaceutically acceptable salts thereof.

12. The method of claim 11, wherein the BTK inhibitor is N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide.

13. The method of claim 1, wherein the BTK inhibitor is ibrutinib (1-[(3R)-3-[4-amino-3-(4-phenoxyphenyl)pyrazolo[3,4-d]pyrimidin-1-yl]piperidin-1-yl]prop-2-en-1-one).

14. The method of claim 1, wherein the BTK inhibitor is Roche BTKi RN486.

15. A method of treating diffuse large B-cell lymphoma (DLBCL) in a subject in need thereof comprising:
administering a therapeutically effective dose of Compound A or a pharmaceutically acceptable salt form thereof to said subject; and
administering a therapeutically effective dose of BTK inhibitor or a pharmaceutically acceptable salt form thereof to said subject.

16. The method of claim 15, wherein the therapeutically effective dose of Compound A is about 50 to 500 mg, and the therapeutically effective dose of BTK inhibitor is about 50 to 500 mg.

17. The method of claim 15, wherein the BTK inhibitor is selected from N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide, Ibrutinib, acalabrutinib, Zanubrutinib, and BTKi RN486.

18. A method of treating Waldenström macroglobulinemia (WM) in a subject in need thereof comprising:
administering a therapeutically effective dose of Compound A or a pharmaceutically acceptable salt form thereof to said subject; and
administering a therapeutically effective dose of BTK inhibitor or a pharmaceutically acceptable salt form thereof to said subject.

19. The method of claim 18, wherein the therapeutically effective dose of Compound A is about 50 to 500 mg, and the therapeutically effective dose of BTK inhibitor is about 50 to 500 mg.

20. The method of claim 18, wherein the BTK inhibitor is selected from N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide, Ibrutinib, acalabrutinib, Zanubrutinib, and BTKi RN486.

19. A method of treating chronic lymphocytic leukemia (CLL) in a subject in need thereof comprising:
administering a therapeutically effective dose of Compound A or a pharmaceutically acceptable salt form thereof to said subject; and
administering a therapeutically effective dose of BTK inhibitor or a pharmaceutically acceptable salt form thereof to said subject.

20. The method of claim 19, wherein the therapeutically effective dose of Compound A is about 50 to 500 mg, and the therapeutically effective dose of BTK inhibitor is about 50 to 500 mg.

21. The method of claim 19, wherein the BTK inhibitor is selected from N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide, Ibrutinib, acalabrutinib, Zanubrutinib, and BTKi RN486.
